(19) United States
(12) Patent Application Publication
(10) Pub. No.: US 2025/0197946 A1
(43) Pub. Date: Jun. 19, 2025

(54) BIOMARKERS FOR COLORECTAL NEOPLASIA

(71) Applicant: Novigenix SA, Epalinges (CH)

(72) Inventors: Laura CIARLONI, Epalinges (CH); Sahar HOSSEINIAN EHRENSBERGER, Epalinges (CH); Noushin HADADI, Epalinges (CH); Victoria WOSIKA, Epalinges (CH); Sylvain MONNIER-BENOIT, Epalinges (CH)

(21) Appl. No.: 18/867,050

(22) PCT Filed: May 22, 2023
(86) PCT No.: PCT/EP2023/063691

§ 371 Date: Nov. 19, 2024

(30) Foreign Application Priority Data
May 20, 2022 (EP) 22174682.9

(51) Int. Cl. C12Q 1/6886; G16H 50/20

(52) U.S. Cl. CPC C12Q 1/6886; G16H 50/20; C12Q 2600/112; C12Q 2600/118; C12Q 2600/158

(57) ABSTRACT

The present invention concerns a method for assessing risk of, detecting, diagnosing, prognosticating, predicting and/or monitoring colorectal neoplasia in a subject, wherein the method comprises measuring the expression level of at least one of the genes disclosed in the present application in a sample from the subject.

### FIELD OF THE INVENTION

The invention relates to biomarkers and to agents specifically binding thereto, for use in assessing risk of, detecting (or screening), diagnosing, prognosticating, predicting and/or monitoring colorectal neoplasia in subjects.

### BACKGROUND OF THE INVENTION

In many diseases and conditions, a favourable outcome of prophylactic and/or therapeutic treatments is strongly correlated with early and/or accurate prediction, diagnosis and/or prognosis of the disease or condition. Therefore, there exists a continuous need for additional and preferably improved means and methods for early and/or accurate prediction, diagnosis and/or prognosis of diseases and conditions to guide the treatment choices.

Colorectal cancer (CRC) is the second most common cancer in Western countries, and represents the second leading cause of cancer-related death (Jemal, A. et al. 2009, Cancer statistics, Cancer J Clin. 59, 225-49). Fortunately, there is evidence that screening of average-risk individuals can result in mortality and incidence reduction by early cancer detection and removal of cancer precursor lesions (Gellad, Z. F. et al. 2010, Gastroenterology, 138, 2177-90). Indeed, the goal of screening programs is the detection of early-stage CRC and advanced adenomas (AA) which are premalignant lesions associated with a high risk of progression to an invasive lesion.

Currently, there is not optimal and universally accepted strategy for CRC screening (Hoff, G. et al. 2010, Gut, 59, 407-14). Fecal-based tests are hampered by their limited sensitivity, whereas colonoscopy constitutes an invasive approach (Quintero, E. et al. 2012, N Engl J Med, 366, 697-706). Therefore, new approaches that can complement and improve current strategies are urgently needed. For example, document WO2013/093635 discloses the use of plasma microRNAs for the detection of early colorectal cancer.

In view of this, there exists a persistent need for additional biomarkers, for detecting conditions associated with colorectal neoplasia, e.g. for discriminating the different stages of said conditions.

### BRIEF DESCRIPTION OF THE FIGURES

FIG. 1: Receiving Operating Characteristics (ROC) analysis showing the performance of the AA classifier on training set (5-fold cross validation) to correctly predict AA.

FIG. 2: ROC analysis showing the performance of the AA classifier on validation set to correctly predict AA.

FIG. 3: ROC analysis showing the performance of the CRC classifier on validation set to correctly predict CRC.

### BRIEF SUMMARY OF THE INVENTION

The present invention relates to a method for assessing risk of, detecting, diagnosing, prognosticating, predicting and/or monitoring colorectal neoplasia in a subject, wherein the method comprises measuring the expression level of at least one of the genes disclosed in any one of Tables 1 to 6 in a sample from the subject.

The present invention relates to a kit for assessing risk of, detecting, diagnosing, prognosticating, predicting and/or monitoring colorectal neoplasia comprising means for measuring the expression level of the genes as defined in the present invention.

The present invention relates to use of at least one gene of selected from the group comprising the genes of Table 1, Table 2, Table 3, Table 4, Table 5 and/or Table 6 in a method for assessing risk of, detecting, diagnosing, prognosticating, predicting and/or monitoring colorectal neoplasia in a subject.

The present invention relates to a device for performing a method of the invention for assessing risk of, detecting, diagnosing, prognosticating, predicting and/or monitoring colorectal neoplasia.

The present invention relates to a computer-implemented method for performing a method of the invention for assessing risk of, detecting, diagnosing, prognosticating, predicting and/or monitoring colorectal neoplasia.

The present invention also relates to the use of a kit for assessing risk of, detecting, diagnosing, prognosticating, predicting and/or monitoring colorectal neoplasia.

### DESCRIPTION OF THE INVENTION

Having conducted extensive experiments and tests, the inventors have identified genes differentially expressed in whole blood and peripheral blood mononuclear cells (PBMC) of subjects having colorectal neoplasia. These new biomarkers are useful for assessing risk of, detecting (or screening), diagnosing, prognosticating, predicting and/or monitoring different forms of colorectal neoplasia, i.e. colorectal cancer (CRC), early stage (stages I and II) colorectal cancer (CRC I-II) and advanced adenomas (AA), especially AA with high grade dysplasia (AA hg).

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application.

Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The articles “a” and “an” are used herein to refer to one or to more than one (i.e., to at least one) of the grammatical object of the article. By way of example, “an element” means one element or more than one element.

As used herein, “at least one” means “one or more”, “two or more”, “three or more”, etc. For example, at least one of the genes disclosed in Tables 1 to 3, includes 2, 3, 4, 5, 6, 7, 8, 9, 10, 11, 12, 13, 14, 15, 16, 17, 18, 19, 20, 25 or more genes.

“About” as used herein when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprise(s)” and “comprising” also encompass the more restricted ones “consist(s)”, “consisting” as well as “consist/consisting essentially of”, respectively.

The term “polynucleotide” as used herein is defined as a chain of nucleotides which can be single-stranded (ss) or double-stranded (ds). Furthermore, nucleic acids are polymers of nucleotides. Thus, “nucleic acids” and “polynucleotides” as used herein are interchangeable and refer to any kind of deoxyribonucleotide (e.g. DNA, cDNA, . . . ) or ribonucleotide (e.g. RNA, mRNA, . . . ) polymer or a combination of deoxyribonucleotide and ribonucleotide (e.g. DNA/RNA) polymer, in linear or circular conformation. One skilled in the art has the general knowledge that nucleic acids are polynucleotides, which can be hydrolyzed into the monomeric “nucleotides.” The monomeric nucleotides can be hydrolyzed into “nucleosides”. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR and the like, and by synthetic means.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “nucleotide sequence encoding an amino acid sequence” includes all nucleotide sequences that are degenerate versions of each other and that encode the same amino acid sequence. The phrase nucleotide sequence that encodes a protein or an RNA or a cDNA may also include introns to the extent that the nucleotide sequence encoding the protein may in some version contain an intron(s).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

In the context of the present invention, measuring the expression level of at least one of the genes includes measuring the level of transcription and/or expression and/or activity level (e.g. at the protein level) of at least one of the genes disclosed in anyone Tables 1 to 6.

“Homologous” or “identical” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous or identical at that position. The percent of homology/identity between two sequences is a function of the number of matching or homologous positions shared by the two sequences divided by the number of positions compared X 100. For example, if 6 of 10 of the positions in two sequences are matched or homologous then the two sequences are 60% homologous/identical. Generally, a comparison is made when two sequences are aligned to give maximum homology/identity.

The term “isolated” or “purified” with reference to a particular component (such as for instance, a protein, polypeptide, peptide or fragment thereof, but also a polynucleotide such as a RNA) generally denotes that such component exists in separation from—for example, has been separated from or prepared in separation from—one or more other components of its natural environment. For instance, an isolated human or animal protein, polypeptide, peptide or fragment exists in separation from a human or animal body where it occurs naturally.

The term “marker” or “biomarker” is widespread in the art and may broadly denote a biological molecule and/or a detectable portion thereof whose qualitative and/or quantitative evaluation in a subject is informative (e.g., risk assessment, predictive, diagnostic and/or prognostic) with respect to one or more aspects of the subject's phenotype and/or genotype, such as, for example, with respect to the status of the subject as to a given disease or condition. In the context of the present invention, a biomarker represents the expression product of at least one of the genes disclosed in any one of Tables 1 to 6 such as at least one polynucleotide (DNA, RNA, cDNAs, amplified RNAs or DNAs), or at least one polypeptide encoded by at least one of the genes disclosed in any one of Tables 1 to 6.

The terms “gene expression signature”, “gene signature” or “biomarker signature” refer, in the context of the invention, to high-performing set or panel of genes useful to distinguish between control (CON) and AA, between control (CON) and CRC as well as between AA and CRC.

The terms “assessing risk of” or “risk assessment”, “detecting” or “detection”, “screening”, “diagnosing” or “diagnosis”, “prognosticating” or “prognosis”, “predicting” or “prediction”, and “monitoring” are commonplace and well-understood in medical and clinical practice.

By means of further explanation and without limitation, “assessing risk of” or “risk assessment” generally refer to an advance declaration, indication or foretelling of a disease or condition in a subject not (yet) having said disease or condition. For example, a risk assessment of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “risk assessment of a disease” in a subject may also particularly mean that the subject is at risk of having said disease (e.g., the risk is significantly increased vis-à-vis a control subject or subject population). In relation to colorectal neoplasia, the risk is e.g. assessed in subjects older than 50 years of age or having a personal or familial history of colorectal neoplasia at age below 50 years.

The terms “diagnosing” or “diagnosis” generally refer to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). As used herein, “diagnosis of a disease” in a subject may particularly mean that the subject has said disease, hence, is diagnosed as having said disease. A subject may be diagnosed as taught herein as not having said disease despite displaying one or more conventional symptoms or signs reminiscent thereof.

In the frame of the present invention, the terms “detecting” or “detection” or “screening” have a similar meaning and encompass both risk assessment and diagnosis, i.e. refer to the process of measuring the level of a biomarker in subjects having or not having symptoms of the disease, potentially in any subject.

It is understood that the detection and measurement of the level of transcription and/or expression and/or activity level of one or more genes of the invention in a sample obtained can be direct or indirect. For example, the abundance levels of a polypeptide can be directly quantitated. Alternatively, the amount of at least one of the genes disclosed in any one of Tables 1 to 6 can be determined indirectly by measuring abundance levels of cDNAs, amplified RNAs or DNAs, or by measuring quantities or activities of RNAs, or other molecules that are indicative of the expression level of the at least one of the genes disclosed in any one of Tables 1 to 6. In one aspect, the detection and measurement of the level of transcription and/or expression and/or activity of at least one of the genes disclosed in any one of Tables 1 to 6 is determined indirectly by measuring abundance levels of cDNAs.

The terms “prognosticating” or “prognosis” generally refer to an anticipation on the progression of a disease or condition and the prospect (e.g., the probability, duration, and/or extent) of recovery. A good prognosis of a disease may generally encompass anticipation of a satisfactory partial or complete recovery from said disease, preferably within an acceptable time period. A good prognosis of said disease may more commonly encompass anticipation of not further worsening or aggravating of the conditions, preferably within a given time period. A poor prognosis of a disease may generally encompass anticipation of a substandard recovery and/or unsatisfactorily slow recovery, or to substantially no recovery or even further worsening of said disease.

The terms “predicting” or “prediction” generally refer to an anticipation on the efficacy/efficiency of a medical or surgical treatment on the progression of a disease or condition and the prospect (e.g., the probability, duration, and/or extent) of recovery. A good prediction may generally encompass anticipation of a satisfactory partial or complete recovery from said disease in response to the treatment, preferably within an acceptable time period. A good prediction may more commonly encompass anticipation of not further worsening or aggravating of the conditions in response to the treatment, preferably within a given time period. A poor prediction may generally encompass anticipation of a substandard recovery and/or unsatisfactorily slow recovery, or to substantially no recovery or even further worsening of said disease in response to the treatment.

The terms “monitor” or “monitoring” generally refer to observe and check the progress of a disease or condition in a subject over a period of time, e.g. to evaluate the response to treatment, or to identify relapse of the disease.

A molecule or analyte, or a group of two or more molecules or analytes, is “measured” in a sample when the presence or absence and/or quantity of said molecule(s) or analyte(s) is detected or determined in the sample, preferably substantially to the exclusion of other molecules and analytes.

The terms “quantity”, “amount” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line expression of the biomarker. These values or ranges can be obtained from a single patient or from a group of patients.

An absolute quantity of a molecule or analyte in a sample may be advantageously expressed as weight or as molar amount, or more commonly as a concentration, e.g., weight per volume or mol per volume.

A relative quantity of a molecule or analyte in a sample may be advantageously expressed as an increase or decrease or as a fold-increase or fold-decrease relative to said another value, such as relative to a reference value as taught herein. Performing a relative comparison between first and second parameters (e.g., first and second quantities) may but need not require to first determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs. the second parameter, without the actual need to first convert the readouts to absolute values of the respective parameters.

In the context of the invention, the terms “differentially expressed genes” (or DGE) refer to genes which level of expression is different (increased or decreased) when comparing two subjects or two groups of subjects differing for a given parameter. Gene expression results in the production of a functional RNA (e.g. messenger RNA or mRNA; microRNA or miRNA; other non-coding RNAs such as, e.g. piwi-associated RNAs, endogenous short-interfering RNAs, lncRNAs, . . . ) or a protein.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The terms “patient,” “subject,” “individual,” and the like are used interchangeably herein, and refer to any animal, or cells thereof whether in vitro or in situ, amenable to the methods described herein. As used herein they typically denote humans, but may also encompass reference to non-human animals, preferably warm-blooded animals, more preferably mammals, such as, e.g., non-human primates, rodents, canines, felines, equines, ovines, porcines, and the like. The term does not denote a particular age or sex. Thus, adult and newborn subjects, whether male or female, are intended to be covered.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced. A disease or disorder is “cured” if the severity of a symptom of the disease or disorder, the frequency with which such a symptom is experienced by a patient, or both, is eliminated.

In the context of the present disclosure, the disease is colorectal neoplasia. In one aspect the colorectal neoplasia is advanced adenoma (AA) or colorectal cancer (CRC).

A “therapeutic” treatment is a treatment administered to a subject who exhibits signs of pathology, for the purpose of diminishing or eliminating those signs.

As used herein, “treating a disease or disorder” means reducing the frequency or severity of at least one sign or symptom of a disease or disorder experienced by a subject. Disease and disorder are used interchangeably herein in the context of treatment.

An “effective amount” of a compound is that amount of compound which is sufficient to provide a beneficial effect to the subject to which the compound is administered. The phrase “therapeutically effective amount,” as used herein, refers to an amount that is sufficient or effective to prevent or treat (delay or prevent the onset of, prevent the progression of, inhibit, decrease or reverse) a disease or disorder or condition, including alleviating symptoms thereof. An “effective amount” of a delivery vehicle is that amount sufficient to effectively bind or deliver a compound.

As used herein and according to a well-accepted medical definition, the term “colorectal neoplasia” includes any neoplasia of the intestinal tract below the small intestine (i.e., the large intestine (colon), including the cecum, ascending colon, transverse colon, descending colon, sigmoid colon, and rectum). The term neoplasia is meant to include a broad spectrum of epithelial-derived tumors ranging from benign growths to invasive cancer. This includes pre-cancer colorectal adenomas and colorectal carcinoma. The terms “adenoma” and “adenomatous polyps” or “colorectal cancer” (CRC) and “colorectal carcinoma” can be used interchangeably.

The adenomas with a higher probability to progress to invasive cancer are defined in the context of this invention as “advanced adenomas” (AA). The advanced adenomas with the highest probability are the advanced adenomas with high grade dysplasia (AA hg).

According to a first aspect, the present invention concerns the use of the genes listed in Tables 1 to 6 as disclosed below (see EXAMPLES), as marker(s) of colorectal neoplasia.

Said genes are identified based through their reference (noted “ID” in the tables) in the Ensembl database in relation to the human reference genome hg38. They are also characterized by their name (noted “Symbol” in the tables) with reference to the protein they encode or putatively encode, as deduced from their sequence homology. This name encompasses such proteins and encoding sequences of any organism where found, and particularly of animals, preferably vertebrates, more preferably mammals, including humans and non-human mammals, even more preferably of humans. The terms particularly encompass such proteins and encoding sequences with a native sequence, i.e., ones of which the primary sequence is the same as that found in or derived from nature. A skilled person understands that native sequences may differ between different species due to genetic divergence between such species. Moreover, the native sequences may differ between or within different individuals of the same species due to normal genetic diversity (variation) within a given species. Also, the native sequences may differ between or even within different individuals of the same species due to post-transcriptional or post-translational modifications.

In the frame of the invention, it has been demonstrated that said genes are markers of colorectal neoplasia. Therefore, they can be used to evaluate the conditions associated (CRC, including early CRC, and/or AA, including AA hg) or to monitor variations thereof.

In particular, a positive result to the claimed method tells that a subject has an increased probability of having colorectal neoplasia compared to the general population.

Whereas the measurement of said marker(s) is easy, especially via in vitro methods performed on a sample of a subject, rapid, reliable and cost-effective, their use can e.g. allow to select and then reduce the number of subjects who should undergo colonoscopy, an in vivo invasive method which allows the visualization of benign or cancerous lesions.

In the frame of the invention, it has been shown that the listed genes are differentially transcribed and/or expressed,, i.e. that there is a correlation between the level of said marker(s) and colorectal neoplasia: the level of expression of the gene(s) can be significantly increased or decreased in a subject having CRC and/or AA, in comparison with the level in healthy subjects, i.e. not suffering from colorectal neoplasia (i.e. level reference).

Differential expression (DE) analysis can be performed using the R software package DESeq2. The package estimates variance-mean dependence in count data and test for differential expression based on a model using the negative binomial distribution. To estimate the value of single genes in separating the different groups from control group, Wald testing can be carried out. The resulting p-values can be adjusted for multiple testing by Benjamini-Hochberg adjustment (FDR). Advantageously, genes with an adjusted p-value<0.01 or p<0.05 are considered statistically significantly differentially expressed.

A differential transcription and/or expression level of the gene(s) can correspond to a downregulated or upregulated expression of said gene(s). In one aspect, the differential transcription and/or expression of the gene(s) corresponds to a downregulated expression of said gene(s).

Preferably, the downregulated differential transcription and/or expression of said gene(s) corresponds to a decrease equal or superior to about 5%, preferably equal or superior to about 20%, more preferably equal or superior to about 40% most preferably equal or superior to about 60%, more preferably equal or superior to about 500%, even more preferably equal or superior to about 1000%, in particular equal or superior to about 5000% when compared to the level of corresponding transcription and/or expression of the gene(s) determined previously, e.g. in a control sample.

In one aspect, the differential transcription and/or expression of the gene(s) corresponds to a upregulated expression of said gene(s). Preferably, the upregulated differential transcription and/or expression of said gene(s) corresponds to an increase equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000% when compared to the level of corresponding transcription and/or expression of the gene(s) determined previously, e.g. in a control sample.

In one aspect, the present invention concerns a method for assessing risk of, detecting (or screening), diagnosing, prognosticating, predicting and/or monitoring colorectal neoplasia in a subject, wherein the method comprises measuring the expression level of at least one of the genes disclosed in any one of Tables 1 to 6 (i.e. Table 1, 2, 3, 4, 5 and/or 6) in a sample from the subject.

One understands that methods of risk assessment, detection (or screening), diagnosis, prognosis, prediction and/or monitoring of diseases or conditions generally comprise an examination phase in which data is collected from and/or about the subject. According to an embodiment, the method of the invention comprises measuring the expression level of at least one of the genes disclosed in any one of Tables 1 to 3 in a sample from the subject.

According to one embodiment, the method of the invention comprises the following steps:
i) measuring the expression level of at least one of the genes disclosed in any one of Tables 1 to 6 in the sample from a subject;
ii) calculating a probability score based on the measurement of step (i), and
iii) comparing the probability score calculated in (ii) with a pre-determined reference score associated to a known risk, diagnosis, prognosis and/or prediction of colorectal neoplasia;
iv) finding a positive or negative deviation or no deviation of the quantity measured in (i) from the reference score; and
v) attributing said finding of positive or negative or no deviation to a particular risk, diagnosis, prognosis and/or prediction of colorectal neoplasia in the subject.

As indicated above, the present methods may employ pre-determined score that corresponds to reference values for the expression level of the genes listed in Tables 1 to 6, which may be established according to known procedures previously employed for other biomarkers.

Such reference values may be established either within (i.e., constituting a step of) or external to (i.e., not constituting a step of) the methods of the present invention as defined herein. Accordingly, any one of the methods taught herein may comprise a step of establishing a reference value for the expression level of the genes, said reference value representing either (a) a risk or a diagnosis of no disease or a good prognosis or prediction for said disease, or (b) a risk or diagnosis of the disease or a poor prognosis or prediction for said disease.

The pre-determined score or reference value can be determined based on a single individual. However, and to be more accurate, the pre-determined score or reference value is preferably determined on a group of subjects (i.e. a population), more preferably composed of at least 10 subjects, or even 500 subjects.

According to one embodiment, the method of the invention is performed on a subject who can be:
i) A subject not having any clinical signs or symptoms of colorectal neoplasia, especially for early detection of the disease or preventive screening; or
ii) A subject suspected to have AA or CRC, especially for confirming the diagnosis and evaluating the stage of the disease;
iii) A subject known to have or have had AA or CRC, especially for monitoring the relapse or evolution of the disease or the efficacy of a treatment;
iv) A subject diagnosed with CRC who need to be assigned or not to an adjuvant or neoadjuvant therapy, especially to determine his/her prognostic risk.

Hence, in various embodiments, the present methods may be used in individuals who have not yet been diagnosed as having said diseases or been without symptoms (for example, preventive screening or risk assessment), or who have been diagnosed as having said diseases, or who are suspected of having said diseases (for example, display one or more symptoms characteristic of said diseases), or who are at risk of developing said diseases (for example, genetic predisposition; presence of one or more developmental, environmental or behavioral risk factors).

According to a specific embodiment and in relation to said methods, the reference value corresponds to the expression level determined in healthy subject(s) or not suffering from colorectal neoplasia. In that case, if the expression level in the tested subject is increased or decreased in comparison to the reference value, it can be concluded that said subject is of risk of developing or suffers from colorectal neoplasia. If no significant difference is observed, than it can be concluded that the tested subject is not of risk of developing or doesn't suffer from colorectal neoplasia.

The methods may also be used to detect various stages of progression or severity of said diseases.

The methods may also be used to detect response to prophylactic or therapeutic treatments or other interventions, i.e. in prediction methods. In the frame of the application, medical treatment or therapy is selected from the group comprising chemotherapeutic agents, targeted therapy drugs, small-molecule drugs, monoclonal antibodies, radiation therapy (e.g. external-beam or internal-beam), immunotherapy (e.g. monoclonal antibodies, CAR T-cell therapy adoptive cell transfer, vaccine, . . . ), endoscopic treatment (e.g. colonoscopy) and/or surgical treatment (e.g. resection of lesions, partial or total colectomy, polypectomy, proctectomy, . . . ).

The methods can furthermore be used to help the medical practitioner in deciding upon worsening, status-quo, partial recovery, or complete recovery of the patient from a disease, resulting in either further treatment or observation or in discharge of the patient, i.e. in prediction or prognosis methods.

In such methods, the reference value can be the expression level measured in:
i) subject(s) responding to a given treatment: if the expression level in the tested subject is increased or decreased in comparison to said reference value, it can be concluded that said subject is likely not to respond to the treatment. If no significant difference is observed, than it can be concluded that the tested subject is likely to respond to the treatment;
ii) subject(s) not responding to a given treatment: if the expression level in the tested subject is increased or decreased in comparison to said reference value, it can be concluded that said subject is likely to respond to the treatment. If no significant difference is observed, than it can be concluded that the tested subject is likely not to respond to the treatment;
iii) subject(s) having a good prognosis: if the expression level in the tested subject is increased or decreased in comparison to said reference value, it can be concluded that said subject is likely to have a poor prognosis. If no significant difference is observed, than it can be concluded that the tested subject is likely to have a good prognosis;
iv) subject(s) having a poor prognosis: if the expression level in the tested subject is increased or decreased in comparison to said reference value, it can be concluded that said subject is likely to have a good prognosis. If no significant difference is observed, than it can be concluded that the tested subject is likely to have a poor prognosis.

The invention further provides a method for monitoring a change in the risk assessment, diagnosis, prognosis and/or prediction of said disease in a subject, comprising:
i) applying methods as taught here above to the subject at one or more successive time points, whereby the risk assessment, diagnosis, prognosis and/or prediction of said disease in the subject is determined at said successive time points;
ii) comparing the risk assessment, diagnosis, prognosis and/or prediction of said disease in the subject at said successive time points as determined in the first step; and
iii) finding the presence or absence of a change between the risk assessment, diagnosis, prognosis and/or prediction of said disease in the subject at said successive time points as determined in the first step.

This aspect allows monitoring the subject's condition over time. This can inter alia allow monitoring in said subject the disease progression, disease aggravation or alleviation, disease recurrence, response to treatment, response to other external or internal factors, conditions, or stressors, etc. Advantageously, the change in the risk assessment, diagnosis, prognosis and/or prediction of said disease in the subject may be monitored in the course of a medical treatment of said subject, preferably a medical treatment aimed at treating said disease. Such monitoring may be comprised, e.g., in decision making whether a patient may be discharged or needs further hospitalization or treatment.

According to another aspect, the method of the invention is used for selecting a cancer therapy or for evaluating a candidate drug.

The terms “sample” or “biological sample” as used herein include any biological specimen obtained from a subject. Samples may include, without limitation, whole blood, plasma, serum, red blood cells, white blood cells (e.g., peripheral blood mononuclear cells), saliva, urine, stool (i.e., faeces), tears, sweat, sebum, nipple aspirate, ductal lavage, tumour exudates, synovial fluid, cerebrospinal fluid, lymph, fine needle aspirate, amniotic fluid, any other bodily fluid, cell lysates, cellular secretion products, inflammation fluid, semen and vaginal secretions. In some aspects, the biological sample also includes extracellular vesicles such as, e.g. exosomes and microvesicles that are two major categories of extracellular vesicles released by almost all cell types and are highly abundant in biological fluids.

Preferred samples may include ones comprising the marker(s) of interest in detectable quantities. In preferred embodiments, the sample may be whole blood or a fractional component thereof such as, e.g., plasma, serum, or a cell pellet. According to a specific embodiment, the sample used in the method according to the invention is whole blood or a fractional component thereof, such as e.g. white blood cells or peripheral blood mononuclear cells (PBMC).

Preferably the sample is readily obtainable by minimally invasive methods.

According to one embodiment, the method of the invention comprises measuring the expression level of at least one of the genes disclosed in any one of Tables 1 to 3 as shown below. According to a specific embodiment, only one gene is chosen among the listed genes. According to another embodiment, a combination of genes is selected. Such a combination can include 2, 3, 4, 5, 6, 7, 8, 9, 10, 11, 12, 13, 14, 15, 16, 17, 18, 19, 20 genes, possibly 25, 30, 35, 40, 45 or even 50 different genes. Said genes, when combined, can be chosen in the same Table (Table 1, 2, 3, 4, 5 or 6) or in different Tables.

The gene(s) of interest can be selected depending on the specific aim of the one performing the method of the invention.

As an example, when the method concerns colorectal neoplasia in general, any gene listed in any of Tables 1 to 6 can be chosen, especially those for which the adjusted p-value is low.

According to a specific embodiment, when the method concerns colorectal cancer (CRC) in general, the genes can be chosen in Tables 1, 2, 4, and 6, preferably in Tables 2 and 4, more preferably in Table 4.

According to a specific embodiment, when the method concerns advanced adenoma (AA), the genes can be chosen in Tables 1, 2, 3 and 5 preferably in Tables 2 and 3, more preferably in Table 3.

According to a specific embodiment, the method of the invention further comprises measuring in the sample of the subject the presence or absence and/or quantity of one or more other biomarkers useful in the context of said disease. Such other biomarker(s) can be a microRNA, a somatic genomic mutation or copy number variation, a methylated genomic locus, an epigenetically modified nucleosome, circulating tumor DNA, a circulating cell-free RNA, a tumor protein.

In the methods taught herein, the expression level of at least one of the genes may be measured, whether directly or indirectly, by any suitable technique such as may be known in the art, i.e. at the gene level or at the protein level.

In one aspect of the methods and kits of the invention, the expression level of the gene is measured by microarray expression profiling, PCR, reverse transcriptase PCR, reverse transcriptase real-time PCR, quantitative real-time PCR, digital PCR, end-point PCR, multiplex end-point PCR, mass spectrometry, in situ hybridization (ISH), multiplex in situ hybridization, next generation nucleic acid sequencing, or a combination of said methods. According to a preferred embodiment, especially in case of a combination of genes, their expression level is measured by next generation RNA sequencing. RNA sequencing can be applied to the whole transcriptome, polyA RNA, or to specific target gene transcripts.

In one aspect, microarrays are used to measure the levels of one or more genes of the invention. An advantage of microarray analysis is that the expression of each of the one or more genes of the invention can be measured simultaneously, and microarrays can be specifically designed to provide a diagnostic expression profile for a particular disease or condition.

As discussed herein, transcripts of the genes of the invention which may be measured by RNA sequencing analysis can be expressed mRNAs or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)+messenger RNA (mRNA) or a fraction thereof, cytoplasmic mRNA, or RNA transcribed from cDNA (i.e., cRNA; see, e.g., U.S. Pat. Nos. 5,545,522, 5,891,636, or 5,716,785). Methods for preparing total and poly(A)+RNA are well known in the art, and are described generally, e.g., in Sambrook, et al., Molecular Cloning: A Laboratory Manual (3rd Edition, 2001) as well as e.g. in Hong, M., Tao, S., Zhang, L. et al. RNA sequencing: new technologies and applications in cancer research. J Hematol Oncol 13, 166 (2020). RNA can be extracted from a cell of interest using guanidinium thiocyanate lysis followed by CsCl centrifugation, a silica gel-based column (e.g., RNeasy (Qiagen, Valencia, Calif.) or StrataPrep (Stratagene, La Jolla, Calif)), or using phenol and chloroform, as known in the art. Poly(A)+RNA can be selected, e.g., by selection with oligo-dT cellulose or, alternatively, by oligo-dT primed reverse transcription of total cellular RNA. RNA can be fragmented by methods known in the art, e.g., by incubation with ZnCl2, to generate fragments of RNA.

In one aspect, total RNA, mRNAs, or nucleic acids derived therefrom (such as cDNA), are isolated from a sample taken from a patient having cancer or a cancer tissue undergoing surgical and/or pharmacological therapies. Genes of the invention that are poorly expressed in particular cells may be enriched using amplification techniques known in the art.

As described above, the polynucleotides can be detectably labeled at one or more nucleotides. Any method known in the art may be used to label the target polynucleotides. Preferably, this labeling incorporates the label uniformly along the length of the RNA, and more preferably, the labeling is carried out at a high degree of efficiency. For example, polynucleotides can be labeled by oligo-dT primed reverse transcription. Random primers (e.g., 9-mers) can be used in reverse transcription to uniformly incorporate labeled nucleotides over the full length of the polynucleotides. Alternatively, random primers may be used in conjunction with PCR methods or T7 promoter-based in vitro transcription methods in order to amplify polynucleotides. The detectable label may be a luminescent label. For example, fluorescent labels, bioluminescent labels, chemiluminescent labels, and colorimetric labels may be used in the practice of the invention. Fluorescent labels that can be used include, but are not limited to, fluorescein, a phosphor, a rhodamine, or a polymethine dye derivative. Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif), and Cy3 or Cy5 (Amersham Pharmacia, Piscataway, N.J.) can be used. Alternatively, the detectable label can be a radiolabeled nucleotide.

In one aspect, the polynucleotide molecules of the genes of the invention from a patient sample are labeled differentially from the corresponding polynucleotide molecules of a reference sample. The reference can comprise mRNAs from a normal biological sample (i.e., control sample, e.g., biopsy from a subject not having a cancer or a cancer tissue undergoing surgical and/or pharmacological therapies) or from a reference biological sample, (e.g., sample from a subject not having a cancer or a cancer tissue undergoing surgical and/or pharmacological therapies).

Nucleic acid hybridization and wash conditions are chosen so that the target polynucleotide molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook, et al., Molecular Cloning: A Laboratory Manual (3rd Edition, 2001). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS). Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

According to another embodiment of the methods and kits of the invention, the expression level of the gene is measured using a binding agent capable of specifically binding to the protein encoded by said gene and/or to fragments thereof, advantageously using an immunoassay technology, such as direct ELISA, indirect ELISA, sandwich ELISA, competitive ELISA, multiplex ELISA, radioimmunoassay (RIA) or ELISPOT technologies, or using a mass spectrometry analysis method or using a chromatography method, or using a combination of said methods. In an embodiment, the binding agent may be an antibody, aptamer, photoaptamer, protein, peptide, peptidomimetic or a small molecule.

According to one aspect of the invention, where colorectal neoplasia is advanced adenoma (AA) or advanced adenoma with high grade dysplasia (hgAA), the method of the invention comprises measuring the expression level of at least one of the genes disclosed in Table 1, Table 2 and/or Table 3.

In one aspect, the genes are selected from those having an AA score superior or equal to 2.

In one aspect, the method comprises measuring the expression level of
i) at least one of the 100 AA top genes, preferably between 2-100 genes, more preferably between 5-100 genes, most preferably between 10-80 genes, even more preferably between 10-50 genes disclosed in Table 2, and/or
ii) at least one of the genes, preferably between 2-84 genes, more preferably between 5-84 genes, most preferably between 10-84 genes, even more preferably between 15-50 genes disclosed in Table 3.

One may easily understand that the conjunction “and” is used to indicate that the method also considers measuring the expression level of at least one gene of Table 2 and at least one gene of Table 3.

According to one aspect of the invention, where colorectal neoplasia is colorectal cancer (CRC), the method of the invention comprises measuring the expression level of at least one of the genes disclosed in Table 1, Table 2, Table 4 and/or Table 6.

In one aspect, the genes are selected from those having a CRC score superior or equal to 15.

In one aspect, the method comprises measuring the expression level of
i) at least one of the 300 CRC top genes, preferably between 2-300 genes, more preferably between 5-250 genes, most preferably between 10-150 genes, even more preferably between 10-100 genes disclosed in Table 2, and/or
ii) at least one of the genes, preferably between 2-69 genes, more preferably between 5-69 genes, most preferably between 10-69 genes, even more preferably between 15-50 genes disclosed in Table 4, and/or
iii) at least one of the genes, preferably between 2-101 genes, more preferably between 5-101 genes, most preferably between 10-80 genes, even more preferably between 10-50 genes disclosed in Table 6.

Again, one may easily understand that the conjunction “and” is used to indicate that the method also considers measuring the expression level of i) at least one gene of Table 2 and at least one gene of Table 3, ii) or at least one gene of Table 2 and at least one gene of Table 6, iii) or at least one gene of Table 4 and at least one gene of Table 6, or iv) at least one gene of Table 2, at least one gene of Table 4 and at least one gene of Table 6.

According to another aspect, the present invention concerns a kit for colorectal neoplasia comprising means for measuring the expression level of the genes as defined above, and its use for said purpose.

According to a preferred embodiment, said kit further comprises a reference control (a reference value) as defined above.

In an embodiment, the mean(s) to measure the expression level of the gene(s) correspond to primers or probes specifically hybridizing to the biomarker(s) of interest, as well as all the reagents required to perform PCR, advantageously reverse transcriptase real-time PCR.

Said kit can also comprise controls, standards and/or calibrators. It can also comprise means for collecting the sample from the subject.

According to another embodiment, said kit comprises means for detecting and/or measuring one or more other biomarkers useful for assessing risk of, detecting, diagnosing, prognosticating, predicting and/or monitoring colorectal neoplasia.

All components may be suitably labelled as known from the skilled person.

Also disclosed are reagents and tools useful for measuring the genes of interest and optionally the one or more other biomarkers concerned herein.

According to another embodiment, said kit further comprises instructions for use. According to another aspect, the present invention encompasses primers or probes specifically hybridizing to the biomarker(s) of interest.

According to another aspect, the present invention also provides the use of at least one gene selected from the group comprising the genes of Table 1, Table 2, Table 3, Table 4, Table 5 and/or Table 6 in a method for assessing risk of, detecting, diagnosing, prognosticating, predicting and/or monitoring colorectal neoplasia in a subject.

According to another aspect, the present invention encompasses a computer-implemented method for implementing a method for assessing risk of, detecting, diagnosing, prognosticating, predicting and/or monitoring colorectal neoplasia in a subject, said computer-implemented method comprising
(i) measuring the expression level in the sample from the subject;
(ii) calculating a probability score based on the measurement of step (i), and
(iii) comparing the score calculated in (ii) with a pre-determined reference score associated to a known risk, diagnosis, prognosis and/or prediction of colorectal neoplasia;
(iv) finding a positive or negative deviation or no deviation of the quantity measured in (i) from the reference score; and
(v) attributing said finding of positive or negative or no deviation to a particular risk, diagnosis, prognosis and/or prediction of colorectal neoplasia in the subject.

According to another aspect, the present invention encompasses a device for performing a method for assessing risk of, detecting, diagnosing, prognosticating, predicting and/or monitoring colorectal neoplasia in a subject, said device comprising.
i) a sample chamber for a test sample collected from a subject;
ii) an assay module in fluid communication with said sample chamber, said assay module comprising means and/or reagents for detecting and/or measuring, directly or indirectly, the expression level of at least one of the genes disclosed in any one of Tables 1 to 6 in said test sample;
iii) means for computing a neoplasia probability score; and
iv) a user interface wherein said user interface relates the neoplasia probability score to detecting colorectal neoplasia in said subject, stratifying colorectal neoplasia or determining the responsiveness to a treatment.

According to another aspect, the present invention also encompasses a method treatment and/or prevention of colorectal neoplasia, the method comprising:
(i) performing a method for assessing risk of, detecting, diagnosing, prognosticating, predicting and/or monitoring colorectal neoplasia described herein, and
(ii) administering a medical treatment or therapy aimed at treating said colorectal neoplasia if the results conclude that the subject is suffering from colorectal neoplasia.

In one aspect, the medical treatment or therapy is selected from the group comprising chemotherapeutic agents, targeted therapy drugs, small-molecule drugs, monoclonal antibodies, radiation therapy (e.g. external-beam or internal-beam), immunotherapy (e.g. monoclonal antibodies, CAR T-cell therapy adoptive cell transfer, vaccine, . . . ) and/or surgical treatment (e.g. resection of lesions, partial or total colectomy, proctectomy, . . . ).

The invention is further described in detail by reference to the following experimental examples. These examples are provided for purposes of illustration only, and are not intended to be limiting unless otherwise specified.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Examples

### Example 1: Whole Blood

### Study Design

RNA-Seq was used to analyze the whole transcriptome in whole-blood samples from colorectal (CRC) patients, advanced adenoma(AA) patients and controls (CON), with the aim of finding biomarker genes able to specifically discriminate CRC and AA from controls.

Control group included colonoscopy verified subjects clear from any colorectal lesions or with benign hyperplastic polyps. The advanced adenoma group included subjects with an adenoma ≥1 cm, or with high grade dysplasia or with a villous component. The CRC group was diagnosed with adenocarcinoma ranging from stages I to IV (AJCC system, 7th edition). Whole-blood samples were prospectively collected at different clinical sites from subjects undergoing a screening, surveillance or diagnostic colonoscopy and from treatment-naïve colorectal cancer patients.

### Samples

Peripheral blood was drawn prior to any polyp or cancer resection or pre-operative chemotherapy. Blood samples were collected into 2×2.5 ml PAXgene Blood RNA Tube (BD Vacutainer® CPT tubes (PreAnalytix, Becton Dickinson, Switzerland). PAXgene Tubes were stored at 4° C. overnight, then at −20° C. or −80° C. according to manufacturer instructions. Automated purification of total RNA was performed on a QIACcube system (Qiagen, Hilden, Germany) with a DNase treatment. RNA concentration was measured by Qubit fluorometer (Thermo Scientific, Waltham, MA, USA) and RNA integrity was analyzed by Agilent 2100 Bioanalyzer (Agilent Technologies, Santa Clara, CA, USA).

### RNA Sequencing

RNA sequencing libraries were prepared from 500 ng of total RNA using the TruSeq Stranded mRNA Library Prep kit (Illumina, San Diego, CA, USA), and enriched of mRNA by polyA selection with oligo d(T) beads. Enriched mRNA are subjected to globin depletion with the Qiagen FastSelect-Globin Kit (Qiagen, Hilden, Germany) following the manufacturers protocol.

The sequencing libraries were multiplexed and loaded on the flowcell on the Illumina NovaSeq 6000 instrument according to manufacturer's instructions. The samples were sequenced using a 2×150 Pair-End configuration at an average of 30 million reads per sample. Image analysis and base calling were conducted by the NovaSeq Control Software on the NovaSeq instrument. Raw sequence data (.bcl files) generated from Illumina NovaSeq was converted into fastq files and de-multiplexed using Illumina bcl2fastq program version 2.17.

### Sequencing and Quantification

Sequencing data analysis was performed with the bcbio-nextgen pipeline for RNA-seq data developed openly on GitHub. Reads were aligned to the human reference genome hg38 and quantified at the transcript level using Salmon in transcripts per million reads (TPM). Transcripts with an average of <1 TPM across all samples of a given clinical group were filtered out, leaving 16827 genes for further downstream analyses.

Quantification was also performed at the gene level using the program featureCounts, expressed as reads per Kilobase of exon per million reads (RPKM). The same filter as the TPM measure was applied.

A discovery set of 428 subjects (192 CON, 62 AA, and 175 CRC) was used to develop gene expression signatures for AA and CRC detection. Full transcriptome profiles were generated by RNA-seq from peripheral whole blood as described above. Univariate differential gene expression analysis and multivariate feature selection methods were applied to the full transcriptome to identify genes able to discriminate between AA or CRC from controls. Results were integrated into scoring system and each gene received 2 scores: one for AA discrimination an done for CRC discrimination. Genes were ranked according to the 2 scores and score cutoffs were applied to identify top differentially expressed genes (cutoff=2 for AA and cutoff=15 for CRC). This resulted in 2029 genes (table 1), of which 1117 were specific for CRC discrimination, 776 for AA discrimination and 136 genes were in common. When among these 2029 genes we considered the top 100 genes for AA discrimination and the top 300 genes for CRC discrimination we identified 398 unique genes (table 2)

### Performance Estimation of AA and CRC Classifier

The 2029 genes were used to train machine learning classifiers to determine high-performing set of genes (gene signatures) to distinguish between CON and AA and between CON and CRC.

A best performing 84-gene signatures for AA and a 69-gene signature for CRC were identified using Novigenix's proprietary LITOseek platform, feature selection pipeline and predictive machine learning algorithms (table 3 and table 4).

To estimate the performance of selected panels of genes, several machine learning algorithms including Logistic Regression, Random Forest and Neural Network were trained to fit models for predicting AA and/or CRC. The predictive models generate a probability score and score thresholds above which a test sample was called positive and below which a test sample was called negative were determined. Analysis of the true positive and true negatives were used to calculate parameters such as specificity (true negative/total control), sensitivity (true positive/total disease), Receiver Operating Characteristics (ROC) curves and Area Under the Curve (AUC), allowing the evaluation of the predictive performance of the generated models. To reduce the risk of model's overfitting, a 5-fold cross validation method was performed on all performance analysis and the generated results were averaged over the 5 performances.

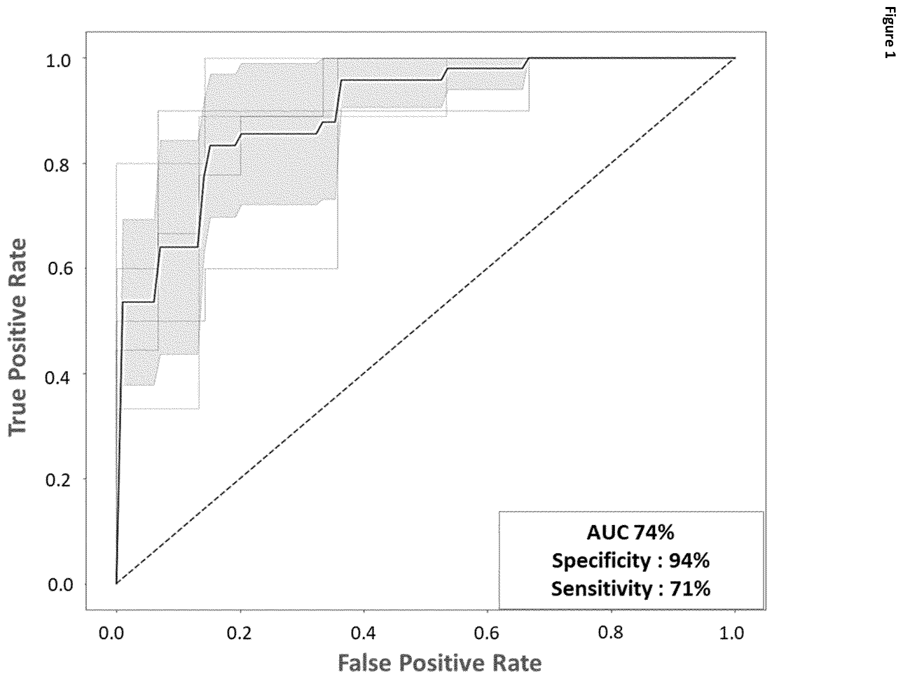

The AA gene classifier showed 71% sensitivity, 94% specificity and an AUC of 74% for AA detection by non-overlapped 5-fold cross validation on the discovery set (FIG. 1).

The predictive accuracy of the gene signature was validated using an independent set of 179 subjects at average risk for CRC and enrolled in screening settings in the EU (75 CON, 26 AA of which 77% with no or low-grade dysplasia, and 78 non-advanced adenoma (NAA)).

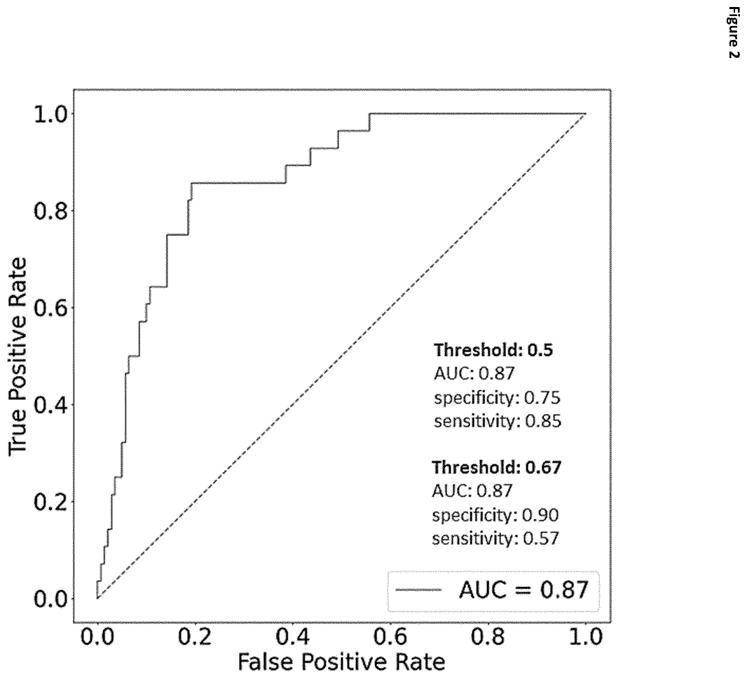

In this cohort, the signature demonstrated a 42.3% sensitivity at 90.5% specificity for detection of AA against those that were negative by colonoscopy (AUC of 0.78) (FIG. 2). Importantly, sensitivity was high for AA located in the proximal colon (53%). The positivity rate on NAA subjects was 15%.

The CRC classifier showed 66% sensitivity, 77% specificity and an AUC of 0.80 for CRC detection by non-overlapped 5-fold cross validation on the discovery set (175 CRC, 191 CON). The predictive accuracy of the gene signature was validated using an independent set of 44 CON and 43 CRC.

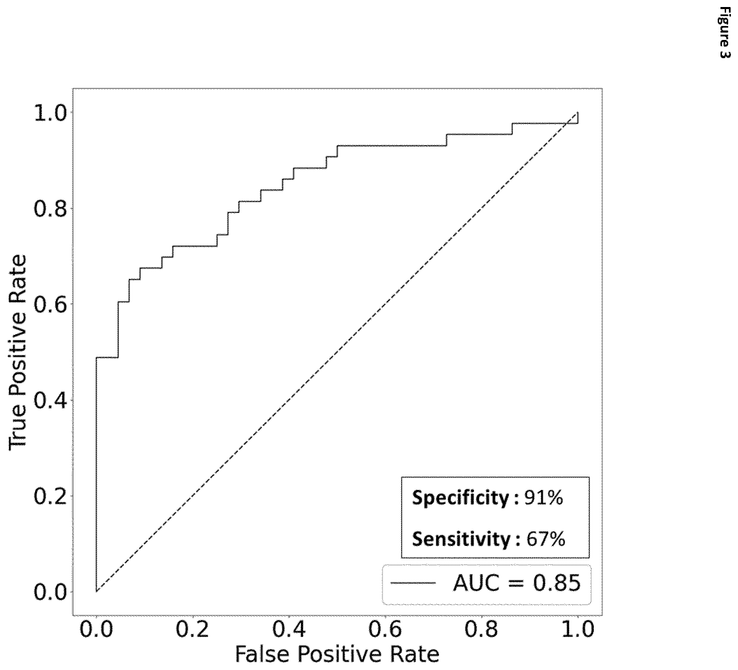

In this cohort, the signature demonstrated a 67% sensitivity at 91% specificity for detection of CRC against those that were negative by colonoscopy (AUC of 0.84) (FIG. 3).

### Example 2

RNA-Seq was used to analyze the whole transcriptome in 275 whole-blood samples from 90 CRC patients, 70 advanced adenoma (AA) patients and 115 controls (CON), with the aim of finding biomarker genes able to specifically discriminate CRC and AA from controls. Potential biomarkers (BMK) were identified through combination of uni- and multi-variate analysis. Multivariant methods, were used to find differentially expressed genes which were not captured using univariant methods. The output of the multivariant methods is different from those of univariant methods, i.e., we do not get pvalue and logFC and instead we obtain importance score. Genes with the highest importance scores were selected.

The main comparisons carried out were CRC vs CON and AA vs CON. To search for specific biomarkers of early cancer or precancerous lesions more at risk to develop into malignant ones, we looked also at stage I-II CRC (earlyCRC) and the high grade dysplasia AA (hgAA) vs CON. In total, four different Differential Expression Analyses (DEA) were performed: CRC (stage I-IV) vs CON, earlyCRC vs CON, AA vs CON, hgAA vs CON.

Uni- and multi-variate analyses were summarized into a gene scoring system. Arbitrary thresholds set on the ranked gene score was used to define the differentially expressed genes (DEGs). Ranking of the gene scores from CRC vs CON DEA identified 1806 DGEs. After filtering for low expressed genes, we identified 912 genes (table 5) that were not overlapping with the 2014 genes identified in the discovery analysis described in example 1.

One CRC classifier using a subset of the 1964 genes showed an AUC of 0.9, 94% specificity and 96% sensitivity for CRC discrimination from controls by cross validation.

One AA classifier using a subset of the 1964 genes showed an AUC of 0.74, 94% specificity and 71% sensitivity for AA discrimination from controls by cross validation.

### Example 3

### Data Set PBMC

### Study Design

RNA-Seq was used to analyze the whole transcriptome in Peripheral Blood Mononuclear Cells (PBMC) samples from 196 CRC patients, 114 advanced adenoma (AA) patients and 226 controls (CON) with the aim of finding biomarker genes able to specifically discriminate CRC and AA from controls.

Control group included subjects clear from any colorectal lesions. The advanced adenoma group included subjects with an adenoma ≥1 cm. The CRC group was diagnosed with an invasive adenocarcinoma ranging from stages I to IV (AJCC system, 7th edition).

Samples were selected from an existing cohort, previously described (Ciarloni L et al. 2016, Clin Cancer Res., 22(18):4604-11). The samples were collected in a multicenter, case-control study in seven Swiss hospitals. The study recruited subjects older than 50 years who were referred by general practitioners for a diagnostic colonoscopy or a screening colonoscopy, or who were scheduled for a colorectal cancer resection.

### Samples

Peripheral blood was drawn prior to any polyp or cancer resection or pre-operative therapy. Blood samples were collected into 4×4 ml BD Vacutainer® CPT tubes (Becton Dickinson, Franklin Lakes, NJ). Filled CPT tubes were kept at room temperature and PBMC separation performed within 6 hours according to manufacturer's instructions. PBMC pellets were resuspended in RNAlater® Solution (Life Technologies, Carlsbad, CA) and stored at −80° C.

### RNA Sequencing

RNA sequencing libraries were prepared from 500 ng of total RNA using the TruSeq Stranded mRNA Library Prep kit (Illumina) including a globin depletion and polyA enrichment. After quality check, two library pools of 80 samples each were created and distinguished by double indexing. Each pool was split into 8 lanes, allocated within 2 sequencing flow cells. This randomization approach was taken to minimize possible biases introduced by the lane or the flow cell effect. Samples were sequenced on the Illumina HiSeq 4000 system (Illumina Inc., Dedham, MA) using a paired-end read length of 75 bp at an average of 30 million reads per sample using standard protocols and reagents.

### Results

We identified 524 candidate biomarkers by combining univariate and multivariate approaches on the first half of the CRC-PBMC cohort (N=295), used as a discovery set. Of the 524, 226 genes were validated as CRC biomarkers in the second half of the CRC-PBMC cohort (N=279). Of those 101 are specific to the PBMC set and do not overlap with the genes identified in example 1 and example 2 (Table 6).
Table 1: List of 2029 genes identified as biomarkers for AA or CRC discrimination. AA and CRC scores rank the top genes in ascending order.
Table 2: List of 398 genes identified as the top100 and top 300 biomarkers for AA or CRC discrimination, respectively. AA and CRC scores rank the top genes in ascending order.
Table 3: Best performing 84 gene signature for AA detection.
Table 4: Best performing 69 gene signature for CRC detection.
Table 5: List of 944 genes included in the 1806 gene panel and not overlapping with the 2024 genes identified in example 1 and reported in Table 1. LogFC indicates the relative abundancy of the gene in the group of interest compared to the control group. The p-value has been adjusted for multiple testing (deseq_adj_p). The genes are ranked according to alphabetical order.
Table 6: List of 101 genes among 226 genes identified in the PBMC dataset as differentially expressed between CRC and CON and not overlapping with the genes identified in example 1 and 2.

TABLE 1

| | | AA | CRC |
|---|---|---|---|
| ensemble ID | gene symbol | score | score |
| ENSG00000128656 | CHN1 | 52.0 | 0.0 |
| ENSG00000289561 | ENSG00000289561 | 51.9 | 2.0 |
| ENSG00000113296 | THBS4 | 49.2 | 0.0 |
| ENSG00000105205 | CLC | 48.7 | 1.1 |
| ENSG00000115602 | IL1RL1 | 47.9 | 2.4 |
| ENSG00000181804 | SLC9A9 | 46.5 | 40.0 |
| ENSG00000263961 | RHEX | 46.3 | 0.0 |
| ENSG00000131203 | IDO1 | 45.5 | 9.7 |
| ENSG00000103056 | SMPD3 | 44.1 | 0.0 |
| ENSG00000273314 | ENSG00000273314 | 43.7 | 0.0 |
| ENSG00000188033 | ZNF490 | 43.3 | 0.0 |
| ENSG00000282608 | ADORA3 | 42.7 | 0.0 |
| ENSG00000169397 | RNASE3 | 42.2 | 0.0 |
| ENSG00000142408 | CACNG8 | 41.8 | 4.2 |
| ENSG00000091181 | IL5RA | 41.5 | 1.2 |
| ENSG00000184208 | C22orf46 | 40.7 | 4.2 |
| ENSG00000143653 | SCCPDH | 40.0 | 0.0 |
| ENSG00000121691 | CAT | 39.8 | 0.0 |
| ENSG00000182557 | SPNS3 | 39.4 | 0.0 |
| ENSG00000175591 | P2RY2 | 39.3 | 0.0 |
| ENSG00000103355 | PRSS33 | 38.7 | 6.2 |
| ENSG00000089091 | DZANK1 | 37.9 | 0.0 |
| ENSG00000124019 | FAM124B | 37.4 | 0.0 |
| ENSG00000235217 | TSPY26P | 36.0 | 0.0 |
| ENSG00000174944 | P2RY14 | 34.3 | 2.2 |
| ENSG00000090470 | PDCD7 | 33.0 | 12.8 |
| ENSG00000255587 | RAB44 | 32.8 | 1.2 |
| ENSG00000128596 | CCDC136 | 32.0 | 0.0 |
| ENSG00000161905 | ALOX15 | 31.3 | 0.0 |
| ENSG00000175048 | ZDHHC14 | 31.0 | 0.0 |
| ENSG00000120279 | MYCT1 | 30.6 | 0.0 |
| ENSG00000138617 | PARP16 | 30.1 | 0.0 |
| ENSG00000105366 | SIGLEC8 | 29.3 | 4.0 |
| ENSG00000232788 | ITGA6-AS1 | 29.0 | 0.0 |
| ENSG00000173166 | RAPH1 | 28.1 | 1.3 |
| ENSG00000112759 | SLC29A1 | 27.4 | 4.9 |
| ENSG00000140263 | SORD | 26.7 | 35.2 |
| ENSG00000276231 | PIK3R6 | 26.3 | 5.9 |
| ENSG00000290017 | ENSG00000290017 | 26.1 | 10.1 |
| ENSG00000186197 | EDARADD | 25.5 | 0.0 |
| ENSG00000013619 | MAMLD1 | 25.3 | 0.0 |
| ENSG00000204165 | CXorf65 | 25.2 | 1.3 |
| ENSG00000239732 | TLR9 | 25.2 | 6.3 |
| ENSG00000048162 | NOP16 | 24.8 | 3.3 |
| ENSG00000133424 | LARGE1 | 24.8 | 6.9 |
| ENSG00000135414 | GDF11 | 24.6 | 19.1 |
| ENSG00000214174 | AMZ2P1 | 24.5 | 0.0 |
| ENSG00000132718 | SYT11 | 24.4 | 1.2 |
| ENSG00000145730 | PAM | 23.5 | 0.0 |
| ENSG00000204444 | APOM | 23.4 | 0.0 |
| ENSG00000130433 | CACNG6 | 23.1 | 0.0 |
| ENSG00000125869 | LAMP5 | 23.1 | 22.1 |
| ENSG00000119673 | ACOT2 | 23.1 | 0.0 |
| ENSG00000144290 | SLC4A10 | 23.0 | 18.9 |
| ENSG00000176903 | PNMA1 | 22.6 | 0.0 |
| ENSG00000119401 | TRIM32 | 22.3 | 6.1 |
| ENSG00000233038 | PTPRN2-AS1 | 22.2 | 0.0 |
| ENSG00000029993 | HMGB3 | 22.2 | 0.0 |
| ENSG00000176083 | ZNF683 | 22.1 | 0.0 |
| ENSG00000185090 | MANEAL | 22.0 | 2.8 |
| ENSG00000011638 | LDAF1 | 21.7 | 0.0 |
| ENSG00000189068 | VSTM1 | 21.6 | 25.7 |
| ENSG00000182195 | LDOC1 | 21.6 | 0.0 |
| ENSG00000119689 | DLST | 21.0 | 0.0 |
| ENSG00000092067 | CEBPE | 21.0 | 0.0 |
| ENSG00000184154 | LRRC51 | 20.7 | 0.0 |
| ENSG00000279693 | ENSG00000279693 | 20.4 | 0.0 |
| ENSG00000184221 | OLIG1 | 20.3 | 0.0 |
| ENSG00000188282 | RUFY4 | 20.3 | 5.0 |
| ENSG00000256128 | LINC00944 | 20.2 | 0.0 |
| ENSG00000166086 | JAM3 | 20.2 | 1.0 |
| ENSG00000026950 | BTN3A1 | 20.2 | 0.0 |
| ENSG00000245904 | BTG1-DT | 20.1 | 21.2 |
| ENSG00000164663 | USP49 | 20.0 | 0.0 |
| ENSG00000123358 | NR4A1 | 19.9 | 0.0 |
| ENSG00000083844 | ZNF264 | 19.8 | 2.3 |
| ENSG00000232098 | ZNF584-DT | 19.3 | 0.0 |
| ENSG00000204472 | AIF1 | 19.3 | 5.5 |
| ENSG00000100448 | CTSG | 19.0 | 0.0 |
| ENSG00000134489 | HRH4 | 18.9 | 1.6 |
| ENSG00000020181 | ADGRA2 | 18.9 | 0.0 |
| ENSG00000196220 | SRGAP3 | 18.9 | 0.0 |
| ENSG00000083814 | ZNF671 | 18.8 | 7.6 |
| ENSG00000184857 | TMEM186 | 18.8 | 0.0 |
| ENSG00000166016 | ABTB2 | 18.8 | 0.0 |
| ENSG00000158050 | DUSP2 | 18.7 | 0.0 |
| ENSG00000150093 | ITGB1 | 18.2 | 0.0 |
| ENSG00000144115 | THNSL2 | 18.2 | 0.0 |
| ENSG00000178538 | CA8 | 18.1 | 0.0 |
| ENSG00000138031 | ADCY3 | 18.0 | 1.1 |
| ENSG00000290043 | ENSG00000290043 | 17.8 | 0.0 |
| ENSG00000169398 | PTK2 | 17.8 | 0.0 |
| ENSG00000005381 | MPO | 17.6 | 0.0 |
| ENSG00000185291 | IL3RA | 17.5 | 0.0 |
| ENSG00000152465 | NMT2 | 17.5 | 18.7 |
| ENSG00000128973 | CLN6 | 17.2 | 0.0 |
| ENSG00000112149 | CD83 | 17.0 | 9.7 |
| ENSG00000008513 | ST3GAL1 | 16.9 | 0.0 |
| ENSG00000099977 | DDT | 16.8 | 0.0 |
| ENSG00000176371 | ZSCAN2 | 16.8 | 0.0 |
| ENSG00000133131 | MORC4 | 16.7 | 16.5 |
| ENSG00000174482 | LINGO2 | 16.6 | 0.0 |
| ENSG00000232810 | TNF | 16.5 | 5.8 |
| ENSG00000168556 | ING2 | 16.5 | 0.0 |
| ENSG00000257497 | GLIPR1-AS1 | 16.5 | 10.6 |
| ENSG00000162174 | ASRGL1 | 16.4 | 0.0 |
| ENSG00000168404 | MLKL | 16.3 | 40.0 |
| ENSG00000148344 | PTGES | 16.2 | 0.0 |
| ENSG00000203705 | TATDN3 | 16.1 | 0.0 |
| ENSG00000150782 | IL18 | 15.8 | 0.0 |
| ENSG00000163995 | ABLIM2 | 15.8 | 0.0 |
| ENSG00000169245 | CXCL10 | 15.7 | 2.4 |
| ENSG00000166035 | LIPC | 15.6 | 3.1 |
| ENSG00000120860 | WASHC3 | 15.5 | 0.0 |
| ENSG00000120738 | EGR1 | 15.4 | 0.0 |
| ENSG00000114315 | HES1 | 15.3 | 0.0 |
| ENSG00000123689 | G0S2 | 15.2 | 5.2 |
| ENSG00000050405 | LIMA1 | 15.1 | 0.0 |
| ENSG00000197077 | KIAA1671 | 15.0 | 0.0 |
| ENSG00000156869 | FRRS1 | 15.0 | 1.3 |
| ENSG00000125740 | FOSB | 14.7 | 0.0 |
| ENSG00000174469 | CNTNAP2 | 14.6 | 2.1 |
| ENSG00000087258 | GNAO1 | 14.6 | 0.0 |
| ENSG00000250696 | ENSG00000250696 | 14.6 | 0.0 |
| ENSG00000223551 | TMSB4XP4 | 14.6 | 0.0 |
| ENSG00000205927 | OLIG2 | 14.5 | 0.0 |
| ENSG00000166619 | BLCAP | 14.4 | 0.0 |
| ENSG00000145348 | TBCK | 14.4 | 0.0 |
| ENSG00000101052 | IFT52 | 14.1 | 0.0 |
| ENSG00000135631 | RAB11FIP5 | 13.9 | 0.0 |
| ENSG00000153885 | KCTD15 | 13.9 | 0.0 |
| ENSG00000167840 | ZNF232 | 13.8 | 26.3 |
| ENSG00000167699 | GLOD4 | 13.8 | 2.4 |
| ENSG00000174136 | RGMB | 13.8 | 3.9 |
| ENSG00000161956 | SENP3 | 13.6 | 0.0 |
| ENSG00000162769 | FLVCR1 | 13.6 | 0.0 |
| ENSG00000149516 | MS4A3 | 13.5 | 0.0 |
| ENSG00000170989 | S1PR1 | 13.5 | 9.0 |
| ENSG00000116455 | WDR77 | 13.5 | 0.0 |
| ENSG00000135605 | TEC | 13.5 | 0.0 |
| ENSG00000178199 | ZC3H12D | 13.5 | 15.0 |
| ENSG00000181027 | FKRP | 13.4 | 0.0 |
| ENSG00000183813 | CCR4 | 13.4 | 0.0 |
| ENSG00000197930 | ERO1A | 13.4 | 0.0 |
| ENSG00000118939 | UCHL3 | 13.2 | 0.0 |
| ENSG00000101417 | PXMP4 | 13.1 | 0.0 |
| ENSG00000186081 | KRT5 | 12.9 | 8.4 |
| ENSG00000113070 | HBEGF | 12.9 | 0.0 |
| ENSG00000081377 | CDC14B | 12.9 | 0.0 |
| ENSG00000188820 | CALHM6 | 12.9 | 0.0 |
| ENSG00000269939 | PCF11-AS1 | 12.7 | 0.0 |
| ENSG00000154269 | ENPP3 | 12.7 | 0.0 |
| ENSG00000174099 | MSRB3 | 12.5 | 2.3 |
| ENSG00000109536 | FRG1 | 12.4 | 0.0 |
| ENSG00000104221 | BRF2 | 12.4 | 0.0 |
| ENSG00000131016 | AKAP12 | 12.3 | 0.0 |
| ENSG00000141560 | FN3KRP | 12.2 | 0.0 |
| ENSG00000155961 | RAB39B | 12.2 | 0.0 |
| ENSG00000280071 | ENSG00000280071 | 12.2 | 7.7 |
| ENSG00000128833 | MYO5C | 12.2 | 0.0 |
| ENSG00000198673 | TAFA2 | 12.1 | 0.0 |
| ENSG00000140307 | GTF2A2 | 12.1 | 0.0 |
| ENSG00000111752 | PHC1 | 12.0 | 28.5 |
| ENSG00000179943 | FIZ1 | 12.0 | 0.0 |
| ENSG00000183625 | CCR3 | 11.9 | 0.0 |
| ENSG00000125650 | PSPN | 11.8 | 0.0 |
| ENSG00000140287 | HDC | 11.7 | 0.0 |
| ENSG00000158481 | CD1C | 11.5 | 1.2 |
| ENSG00000132207 | SLX1A | 11.4 | 0.0 |
| ENSG00000163751 | CPA3 | 11.4 | 0.0 |
| ENSG00000146535 | GNA12 | 11.3 | 0.0 |
| ENSG00000146833 | TRIM4 | 11.3 | 0.0 |
| ENSG00000128567 | PODXL | 11.2 | 0.0 |
| ENSG00000284776 | ENSG00000284776 | 11.2 | 1.4 |
| ENSG00000106245 | BUD31 | 11.1 | 2.6 |
| ENSG00000054654 | SYNE2 | 11.1 | 4.9 |
| ENSG00000155906 | RMND1 | 11.1 | 0.0 |
| ENSG00000116016 | EPAS1 | 11.0 | 21.3 |
| ENSG00000141084 | RANBP10 | 11.0 | 4.0 |
| ENSG00000142794 | NBPF3 | 10.9 | 0.0 |
| ENSG00000246582 | TNFRSF10A-DT | 10.9 | 3.4 |
| ENSG00000240403 | KIR3DL2 | 10.9 | 0.0 |
| ENSG00000088543 | C3orf18 | 10.8 | 0.0 |
| ENSG00000188549 | CCDC9B | 10.7 | 0.0 |
| ENSG00000175556 | LONRF3 | 10.7 | 1.5 |
| ENSG00000213760 | ATP6V1G2 | 10.6 | 1.6 |
| ENSG00000169385 | RNASE2 | 10.6 | 0.0 |
| ENSG00000163348 | PYGO2 | 10.5 | 4.8 |
| ENSG00000167768 | KRT1 | 10.5 | 0.0 |
| ENSG00000091640 | SPAG7 | 10.5 | 0.0 |
| ENSG00000163661 | PTX3 | 10.4 | 0.0 |
| ENSG00000288838 | ENSG00000288838 | 10.4 | 43.6 |
| ENSG00000254415 | SIGLEC14 | 10.3 | 0.0 |
| ENSG00000268480 | NFILZ | 10.3 | 0.0 |
| ENSG00000144214 | LYG1 | 10.3 | 0.0 |
| ENSG00000120262 | CCDC170 | 10.3 | 0.0 |
| ENSG00000151148 | UBE3B | 10.2 | 1.2 |
| ENSG00000170854 | RIOX2 | 10.2 | 20.2 |
| ENSG00000181104 | F2R | 10.2 | 1.5 |
| ENSG00000176945 | MUC20 | 10.2 | 0.0 |
| ENSG00000140262 | TCF12 | 10.1 | 1.2 |
| ENSG00000159202 | UBE2Z | 10.0 | 7.6 |
| ENSG00000185436 | IFNLR1 | 10.0 | 0.0 |
| ENSG00000149328 | GLB1L2 | 9.9 | 0.0 |
| ENSG00000107104 | KANK1 | 9.9 | 4.6 |
| ENSG00000174684 | B4GAT1 | 9.8 | 5.3 |
| ENSG00000261094 | ENSG00000261094 | 9.8 | 0.0 |
| ENSG00000257027 | ENSG00000257027 | 9.8 | 1.8 |
| ENSG00000138758 | SEPTIN11 | 9.8 | 0.0 |
| ENSG00000116237 | ICMT | 9.7 | 0.0 |
| ENSG00000183134 | PTGDR2 | 9.7 | 1.8 |
| ENSG00000161888 | SPC24 | 9.6 | 0.0 |
| ENSG00000148219 | ASTN2 | 9.6 | 0.0 |
| ENSG00000198520 | ARMH1 | 9.6 | 0.0 |
| ENSG00000170345 | FOS | 9.6 | 2.6 |
| ENSG00000157017 | GHRL | 9.5 | 0.0 |
| ENSG00000137959 | IFI44L | 9.4 | 18.4 |
| ENSG00000051596 | THOC3 | 9.4 | 0.0 |
| ENSG00000184867 | ARMCX2 | 9.3 | 8.8 |
| ENSG00000140416 | TPM1 | 9.3 | 34.1 |
| ENSG00000088727 | KIF9 | 9.3 | 0.0 |
| ENSG00000047056 | WDR37 | 9.2 | 2.9 |
| ENSG00000005187 | ACSM3 | 9.2 | 0.0 |
| ENSG00000137266 | SLC22A23 | 9.2 | 0.0 |
| ENSG00000198837 | DENND4B | 9.2 | 0.0 |
| ENSG00000246363 | LINC02458 | 9.2 | 0.0 |
| ENSG00000189241 | TSPYL1 | 9.2 | 12.8 |
| ENSG00000168303 | MPLKIP | 9.1 | 0.0 |
| ENSG00000104312 | RIPK2 | 9.0 | 3.8 |
| ENSG00000143590 | EFNA3 | 9.0 | 0.0 |
| ENSG00000228486 | C2orf92 | 8.8 | 2.3 |
| ENSG00000213626 | LBH | 8.8 | 25.4 |
| ENSG00000233387 | IATPR | 8.7 | 0.0 |
| ENSG00000182183 | SHISAL2A | 8.7 | 8.9 |
| ENSG00000115919 | KYNU | 8.7 | 2.6 |
| ENSG00000276234 | TADA2A | 8.6 | 7.6 |
| ENSG00000106785 | TRIM14 | 8.6 | 0.0 |
| ENSG00000139168 | ZCRB1 | 8.6 | 0.0 |
| ENSG00000113924 | HGD | 8.6 | 0.0 |
| ENSG00000152556 | PFKM | 8.5 | 0.0 |
| ENSG00000120158 | RCL1 | 8.5 | 0.0 |
| ENSG00000226179 | LINC00685 | 8.5 | 0.0 |
| ENSG00000137726 | FXYD6 | 8.4 | 1.2 |
| ENSG00000177374 | HIC1 | 8.4 | 0.0 |
| ENSG00000269378 | ITGB1P1 | 8.3 | 0.0 |
| ENSG00000122224 | LY9 | 8.2 | 20.7 |
| ENSG00000120910 | PPP3CC | 8.2 | 3.6 |
| ENSG00000141428 | C18orf21 | 8.2 | 0.0 |
| ENSG00000196821 | ILRUN | 8.2 | 0.0 |
| ENSG00000176049 | JAKMIP2 | 8.2 | 0.0 |
| ENSG00000151327 | FAM177A1 | 8.2 | 0.0 |
| ENSG00000132465 | JCHAIN | 8.2 | 0.0 |
| ENSG00000135114 | OASL | 8.1 | 30.3 |
| ENSG00000145029 | NICN1 | 8.1 | 0.0 |
| ENSG00000168434 | COG7 | 8.0 | 0.0 |
| ENSG00000122203 | KIAA1191 | 8.0 | 0.0 |
| ENSG00000139531 | SUOX | 8.0 | 5.2 |
| ENSG00000117748 | RPA2 | 8.0 | 0.0 |
| ENSG00000160439 | RDH13 | 8.0 | 0.0 |
| ENSG00000056050 | HPF1 | 7.9 | 0.0 |
| ENSG00000215472 | RPL17-C18orf32 | 7.9 | 0.0 |
| ENSG00000146425 | DYNLT1 | 7.9 | 38.8 |
| ENSG00000146674 | IGFBP3 | 7.8 | 0.0 |
| ENSG00000239672 | NME1 | 7.8 | 0.0 |
| ENSG00000170627 | GTSF1 | 7.8 | 8.0 |
| ENSG00000109099 | PMP22 | 7.7 | 1.6 |
| ENSG00000266338 | NBPF15 | 7.7 | 8.4 |
| ENSG00000125445 | MRPS7 | 7.7 | 0.0 |
| ENSG00000102317 | RBM3 | 7.7 | 4.0 |
| ENSG00000171657 | GPR82 | 7.7 | 0.0 |
| ENSG00000124191 | TOX2 | 7.7 | 0.0 |
| ENSG00000276728 | ENSG00000276728 | 7.7 | 12.9 |
| ENSG00000181007 | ZFP82 | 7.6 | 2.3 |
| ENSG00000111229 | ARPC3 | 7.6 | 11.4 |
| ENSG00000163687 | DNASE1L3 | 7.5 | 31.7 |
| ENSG00000151376 | ME3 | 7.4 | 0.0 |
| ENSG00000171467 | ZNF318 | 7.4 | 6.5 |
| ENSG00000150687 | PRSS23 | 7.4 | 0.0 |
| ENSG00000171101 | SIGLEC17P | 7.4 | 3.8 |
| ENSG00000117595 | IRF6 | 7.4 | 0.0 |
| ENSG00000273824 | ENSG00000273824 | 7.4 | 1.2 |
| ENSG00000112167 | SAYSD1 | 7.3 | 0.0 |
| ENSG00000130755 | GMFG | 7.3 | 7.9 |
| ENSG00000012232 | EXTL3 | 7.3 | 6.3 |
| ENSG00000154451 | GBP5 | 7.3 | 28.4 |
| ENSG00000111328 | CDK2AP1 | 7.3 | 0.0 |
| ENSG00000127125 | PPCS | 7.3 | 0.0 |
| ENSG00000197345 | MRPL21 | 7.2 | 0.0 |
| ENSG00000008277 | ADAM22 | 7.2 | 0.0 |
| ENSG00000137965 | IFI44 | 7.2 | 19.5 |
| ENSG00000095794 | CREM | 7.2 | 0.0 |
| ENSG00000186462 | NAP1L2 | 7.1 | 0.0 |
| ENSG00000238121 | LINC00426 | 7.1 | 1.3 |
| ENSG00000062582 | MRPS24 | 7.1 | 0.0 |
| ENSG00000179348 | GATA2 | 7.1 | 0.0 |
| ENSG00000163521 | GLB1L | 7.1 | 0.0 |
| ENSG00000144645 | OSBPL10 | 7.1 | 20.6 |
| ENSG00000198960 | ARMCX6 | 7.1 | 0.0 |
| ENSG00000138796 | HADH | 7.1 | 5.4 |
| ENSG00000073849 | ST6GAL1 | 7.0 | 6.6 |
| ENSG00000250510 | GPR162 | 7.0 | 0.0 |
| ENSG00000122025 | FLT3 | 7.0 | 0.0 |
| ENSG00000153064 | BANK1 | 7.0 | 42.9 |
| ENSG00000132704 | FCRL2 | 7.0 | 8.3 |
| ENSG00000153029 | MR1 | 7.0 | 34.7 |
| ENSG00000255833 | TIFAB | 7.0 | 0.0 |
| ENSG00000148803 | FUOM | 7.0 | 0.0 |
| ENSG00000135622 | SEMA4F | 7.0 | 0.0 |
| ENSG00000164182 | NDUFAF2 | 7.0 | 0.0 |
| ENSG00000180884 | ZNF792 | 7.0 | 0.0 |
| ENSG00000173950 | XXYLT1 | 6.9 | 10.9 |
| ENSG00000204634 | TBC1D8 | 6.9 | 6.3 |
| ENSG00000187134 | AKR1C1 | 6.9 | 0.0 |
| ENSG00000144567 | RETREG2 | 6.8 | 41.7 |
| ENSG00000116717 | GADD45A | 6.8 | 42.4 |
| ENSG00000162512 | SDC3 | 6.8 | 3.5 |
| ENSG00000226979 | LTA | 6.8 | 0.0 |
| ENSG00000151748 | SAV1 | 6.7 | 4.1 |
| ENSG00000047849 | MAP4 | 6.7 | 0.0 |
| ENSG00000279765 | ENSG00000279765 | 6.7 | 0.0 |
| ENSG00000074621 | SLC24A1 | 6.6 | 1.2 |
| ENSG00000090339 | ICAM1 | 6.6 | 14.6 |
| ENSG00000141034 | GID4 | 6.6 | 0.0 |
| ENSG00000164402 | SEPTIN8 | 6.6 | 0.0 |
| ENSG00000117597 | UTP25 | 6.6 | 8.0 |
| ENSG00000164011 | ZNF691 | 6.6 | 0.0 |
| ENSG00000125450 | NUP85 | 6.6 | 0.0 |
| ENSG00000247796 | MOCS2-DT | 6.6 | 0.0 |
| ENSG00000166847 | DCTN5 | 6.5 | 0.0 |
| ENSG00000169413 | RNASE6 | 6.5 | 0.0 |
| ENSG00000126016 | AMOT | 6.4 | 14.2 |
| ENSG00000073756 | PTGS2 | 6.4 | 0.0 |
| ENSG00000168894 | RNF181 | 6.4 | 4.2 |
| ENSG00000049089 | COL9A2 | 6.4 | 1.5 |
| ENSG00000139990 | DCAF5 | 6.4 | 0.0 |
| ENSG00000110200 | ANAPC15 | 6.4 | 0.0 |
| ENSG00000196914 | ARHGEF12 | 6.4 | 1.2 |
| ENSG00000272374 | ENSG00000272374 | 6.4 | 7.6 |
| ENSG00000166387 | PPFIBP2 | 6.4 | 0.0 |
| ENSG00000158887 | MPZ | 6.3 | 0.0 |
| ENSG00000132326 | PER2 | 6.3 | 4.5 |
| ENSG00000110318 | CEP126 | 6.3 | 2.6 |
| ENSG00000049449 | RCN1 | 6.3 | 0.0 |
| ENSG00000213619 | NDUFS3 | 6.2 | 0.0 |
| ENSG00000265118 | ENSG00000265118 | 6.1 | 0.0 |
| ENSG00000197128 | ZNF772 | 6.1 | 0.0 |
| ENSG00000114859 | CLCN2 | 6.1 | 0.0 |
| ENSG00000005075 | POLR2J | 6.0 | 0.0 |
| ENSG00000015676 | NUDCD3 | 6.0 | 0.0 |
| ENSG00000135940 | COX5B | 6.0 | 0.0 |
| ENSG00000157833 | GAREM2 | 6.0 | 0.0 |
| ENSG00000103365 | GGA2 | 6.0 | 19.9 |
| ENSG00000154237 | LRRK1 | 5.9 | 0.0 |
| ENSG00000188522 | FAM83G | 5.9 | 0.0 |
| ENSG00000257167 | TMPO-AS1 | 5.9 | 0.0 |
| ENSG00000288082 | ENSG00000288082 | 5.9 | 0.0 |
| ENSG00000135905 | DOCK10 | 5.9 | 1.2 |
| ENSG00000198018 | ENTPD7 | 5.9 | 0.0 |
| ENSG00000109390 | NDUFC1 | 5.8 | 0.0 |
| ENSG00000164167 | LSM6 | 5.8 | 0.0 |
| ENSG00000166452 | AKIP1 | 5.8 | 0.0 |
| ENSG00000188917 | TRMT2B | 5.8 | 25.7 |
| ENSG00000158715 | SLC45A3 | 5.7 | 0.0 |
| ENSG00000162722 | TRIM58 | 5.7 | 53.4 |
| ENSG00000111186 | WNT5B | 5.7 | 0.0 |
| ENSG00000163399 | ATP1A1 | 5.7 | 0.0 |
| ENSG00000174173 | TRMT10C | 5.6 | 1.2 |
| ENSG00000111674 | ENO2 | 5.6 | 15.4 |
| ENSG00000143155 | TIPRL | 5.6 | 0.0 |
| ENSG00000143093 | STRIP1 | 5.5 | 0.0 |
| ENSG00000165646 | SLC18A2 | 5.5 | 1.3 |
| ENSG00000110987 | BCL7A | 5.5 | 38.6 |
| ENSG00000100227 | POLDIP3 | 5.5 | 0.0 |
| ENSG00000091972 | CD200 | 5.5 | 15.9 |
| ENSG00000189362 | NEMP2 | 5.4 | 2.8 |
| ENSG00000259162 | ENSG00000259162 | 5.4 | 42.1 |
| ENSG00000101337 | TM9SF4 | 5.4 | 0.0 |
| ENSG00000159461 | AMFR | 5.4 | 0.0 |
| ENSG00000214900 | LINC01588 | 5.4 | 17.7 |
| ENSG00000114902 | SPCS1 | 5.4 | 0.0 |
| ENSG00000283149 | ENSG00000283149 | 5.4 | 0.0 |
| ENSG00000244607 | CCDC13 | 5.3 | 0.0 |
| ENSG00000117614 | SYF2 | 5.3 | 0.0 |
| ENSG00000112031 | MTRF1L | 5.3 | 0.0 |
| ENSG00000124201 | ZNFX1 | 5.3 | 4.0 |
| ENSG00000120278 | PLEKHG1 | 5.3 | 0.0 |
| ENSG00000163993 | S100P | 5.3 | 5.8 |
| ENSG00000266074 | BAHCC1 | 5.3 | 0.0 |
| ENSG00000233392 | UICLM | 5.3 | 0.0 |
| ENSG00000103494 | RPGRIP1L | 5.3 | 0.0 |
| ENSG00000282499 | TRBV25-1 | 5.3 | 13.6 |
| ENSG00000154856 | APCDD1 | 5.3 | 0.0 |
| ENSG00000187922 | LCN10 | 5.2 | 24.0 |
| ENSG00000171643 | S100Z | 5.2 | 0.0 |
| ENSG00000151715 | TMEM45B | 5.2 | 0.0 |
| ENSG00000158526 | TSR2 | 5.2 | 0.0 |
| ENSG00000121552 | CSTA | 5.2 | 8.6 |
| ENSG00000171853 | TRAPPC12 | 5.2 | 0.0 |
| ENSG00000130349 | MTRES1 | 5.2 | 0.0 |
| ENSG00000138615 | CILP | 5.2 | 4.9 |
| ENSG00000112486 | CCR6 | 5.1 | 37.5 |
| ENSG00000110090 | CPT1A | 5.1 | 5.1 |
| ENSG00000152766 | ANKRD22 | 5.1 | 33.2 |
| ENSG00000184979 | USP18 | 5.1 | 7.0 |
| ENSG00000275464 | ENSG00000275464 | 5.0 | 5.9 |
| ENSG00000286342 | ENSG00000286342 | 5.0 | 9.0 |
| ENSG00000159388 | BTG2 | 5.0 | 0.0 |
| ENSG00000177410 | ZFAS1 | 5.0 | 0.0 |
| ENSG00000156738 | MS4A1 | 5.0 | 37.0 |
| ENSG00000151366 | NDUFC2 | 5.0 | 0.0 |
| ENSG00000161692 | DBF4B | 5.0 | 0.0 |
| ENSG00000112304 | ACOT13 | 5.0 | 0.0 |
| ENSG00000028137 | TNFRSF1B | 5.0 | 3.6 |
| ENSG00000187243 | MAGED4B | 4.9 | 1.1 |
| ENSG00000065308 | TRAM2 | 4.9 | 0.0 |
| ENSG00000155090 | KLF10 | 4.9 | 0.0 |
| ENSG00000142512 | SIGLEC10 | 4.9 | 0.0 |
| ENSG00000120071 | KANSL1 | 4.9 | 0.0 |
| ENSG00000119729 | RHOQ | 4.9 | 0.0 |
| ENSG00000078140 | UBE2K | 4.9 | 0.0 |
| ENSG00000164330 | EBF1 | 4.9 | 18.0 |
| ENSG00000128253 | RFPL2 | 4.8 | 0.0 |
| ENSG00000125775 | SDCBP2 | 4.8 | 0.0 |
| ENSG00000104918 | RETN | 4.8 | 3.2 |
| ENSG00000204310 | AGPAT1 | 4.8 | 11.4 |
| ENSG00000120129 | DUSP1 | 4.8 | 8.5 |
| ENSG00000169871 | TRIM56 | 4.8 | 4.8 |
| ENSG00000148290 | SURF1 | 4.8 | 3.8 |
| ENSG00000143420 | ENSA | 4.8 | 0.0 |
| ENSG00000181004 | BBS12 | 4.8 | 0.0 |
| ENSG00000110108 | TMEM109 | 4.8 | 0.0 |
| ENSG00000147164 | SNX12 | 4.8 | 0.0 |
| ENSG00000143363 | PRUNE1 | 4.8 | 2.4 |
| ENSG00000173436 | MICOS10 | 4.8 | 0.0 |
| ENSG00000168792 | ABHD15 | 4.8 | 0.0 |
| ENSG00000278053 | DDX52 | 4.7 | 0.0 |
| ENSG00000152256 | PDK1 | 4.7 | 0.0 |
| ENSG00000131401 | NAPSB | 4.7 | 0.0 |
| ENSG00000198353 | HOXC4 | 4.7 | 0.0 |
| ENSG00000198585 | NUDT16 | 4.7 | 13.3 |
| ENSG00000119705 | SLIRP | 4.7 | 0.0 |
| ENSG00000149489 | ROM1 | 4.6 | 0.0 |
| ENSG00000182240 | BACE2 | 4.6 | 0.0 |
| ENSG00000112294 | ALDH5A1 | 4.6 | 0.0 |
| ENSG00000279170 | TSTD3 | 4.6 | 0.0 |
| ENSG00000224137 | LINC01857 | 4.6 | 21.3 |
| ENSG00000266028 | SRGAP2 | 4.6 | 4.7 |
| ENSG00000111331 | OAS3 | 4.6 | 13.3 |
| ENSG00000239839 | DEFA3 | 4.6 | 1.1 |
| ENSG00000115484 | CCT4 | 4.6 | 1.2 |
| ENSG00000158292 | GPR153 | 4.6 | 0.0 |
| ENSG00000242485 | MRPL20 | 4.6 | 0.0 |
| ENSG00000003249 | DBNDD1 | 4.5 | 0.0 |
| ENSG00000103091 | WDR59 | 4.5 | 2.4 |
| ENSG00000144749 | LRIG1 | 4.5 | 14.0 |
| ENSG00000149131 | SERPING1 | 4.5 | 31.9 |
| ENSG00000140105 | WARS1 | 4.5 | 16.4 |
| ENSG00000105492 | SIGLEC6 | 4.5 | 0.0 |
| ENSG00000267106 | ZNF561-AS1 | 4.5 | 0.0 |
| ENSG00000114648 | KLHL18 | 4.4 | 2.4 |
| ENSG00000110700 | RPS13 | 4.4 | 4.8 |
| ENSG00000268758 | ADGRE4P | 4.4 | 4.7 |
| ENSG00000168394 | TAP1 | 4.4 | 46.7 |
| ENSG00000170581 | STAT2 | 4.4 | 21.9 |
| ENSG00000182578 | CSF1R | 4.4 | 0.0 |
| ENSG00000185722 | ANKFY1 | 4.4 | 0.0 |
| ENSG00000228253 | MT-ATP8 | 4.4 | 0.0 |
| ENSG00000243696 | ENSG00000243696 | 4.4 | 0.0 |
| ENSG00000136816 | TOR1B | 4.4 | 37.1 |
| ENSG00000141664 | ZCCHC2 | 4.4 | 20.0 |
| ENSG00000115758 | ODC1 | 4.4 | 3.9 |
| ENSG00000170921 | TANC2 | 4.4 | 0.0 |
| ENSG00000183520 | UTP11 | 4.4 | 0.0 |
| ENSG00000125977 | EIF2S2 | 4.4 | 0.0 |
| ENSG00000111335 | OAS2 | 4.4 | 3.4 |
| ENSG00000147234 | FRMPD3 | 4.4 | 0.0 |
| ENSG00000117322 | CR2 | 4.3 | 14.8 |
| ENSG00000104133 | SPG11 | 4.3 | 8.4 |
| ENSG00000171490 | RSL1D1 | 4.3 | 20.5 |
| ENSG00000166770 | ZNF667-AS1 | 4.3 | 19.3 |
| ENSG00000175215 | CTDSP2 | 4.3 | 0.0 |
| ENSG00000130821 | SLC6A8 | 4.3 | 31.9 |
| ENSG00000187474 | FPR3 | 4.3 | 0.0 |
| ENSG00000155367 | PPM1J | 4.3 | 2.7 |
| ENSG00000152229 | PSTPIP2 | 4.3 | 52.2 |
| ENSG00000196150 | ZNF250 | 4.3 | 0.0 |
| ENSG00000113448 | PDE4D | 4.2 | 3.2 |
| ENSG00000119922 | IFIT2 | 4.2 | 27.6 |
| ENSG00000204267 | TAP2 | 4.2 | 20.0 |
| ENSG00000198771 | RCSD1 | 4.2 | 0.0 |
| ENSG00000107736 | CDH23 | 4.2 | 0.0 |
| ENSG00000138430 | OLA1 | 4.2 | 0.0 |
| ENSG00000105185 | PDCD5 | 4.2 | 0.0 |
| ENSG00000163702 | IL17RC | 4.2 | 0.0 |
| ENSG00000254860 | TMEM9B-AS1 | 4.2 | 0.0 |
| ENSG00000215883 | CYB5RL | 4.1 | 0.0 |
| ENSG00000289027 | ENSG00000289027 | 4.1 | 0.0 |
| ENSG00000134061 | CD180 | 4.1 | 14.5 |
| ENSG00000168016 | TRANK1 | 4.1 | 0.0 |
| ENSG00000254635 | WAC-AS1 | 4.1 | 0.0 |
| ENSG00000256053 | COA8 | 4.1 | 0.0 |
| ENSG00000067369 | TP53BP1 | 4.1 | 0.0 |
| ENSG00000013374 | NUB1 | 4.1 | 2.4 |
| ENSG00000137200 | CMTR1 | 4.1 | 0.0 |
| ENSG00000233527 | ZNF529-AS1 | 4.1 | 0.0 |
| ENSG00000001630 | CYP51A1 | 4.1 | 0.0 |
| ENSG00000115129 | TP53I3 | 4.0 | 7.2 |
| ENSG00000108474 | PIGL | 4.0 | 24.0 |
| ENSG00000157601 | MX1 | 4.0 | 11.6 |
| ENSG00000188822 | CNR2 | 4.0 | 1.5 |
| ENSG00000101882 | NKAP | 4.0 | 0.0 |
| ENSG00000168036 | CTNNB1 | 4.0 | 4.6 |
| ENSG00000183864 | TOB2 | 4.0 | 0.0 |
| ENSG00000173193 | PARP14 | 4.0 | 35.2 |
| ENSG00000173821 | RNF213 | 4.0 | 23.3 |
| ENSG00000182551 | ADI1 | 4.0 | 0.0 |
| ENSG00000008394 | MGST1 | 3.9 | 2.8 |
| ENSG00000100628 | ASB2 | 3.9 | 0.0 |
| ENSG00000112419 | PHACTR2 | 3.9 | 0.0 |
| ENSG00000183486 | MX2 | 3.9 | 28.0 |
| ENSG00000060642 | PIGV | 3.9 | 4.7 |
| ENSG00000144231 | POLR2D | 3.9 | 2.4 |
| ENSG00000216895 | RBM33-DT | 3.9 | 1.3 |
| ENSG00000164346 | NSA2 | 3.9 | 1.2 |
| ENSG00000186625 | KATNA1 | 3.9 | 0.0 |
| ENSG00000263563 | UBBP4 | 3.9 | 11.3 |
| ENSG00000086475 | SEPHS1 | 3.9 | 8.2 |
| ENSG00000267751 | BSG-AS1 | 3.9 | 1.4 |
| ENSG00000106554 | CHCHD3 | 3.9 | 0.0 |
| ENSG00000021355 | SERPINB1 | 3.8 | 32.3 |
| ENSG00000204070 | SYS1 | 3.8 | 2.6 |
| ENSG00000214046 | SMIM7 | 3.8 | 1.5 |
| ENSG00000108826 | MRPL27 | 3.8 | 0.0 |
| ENSG00000139624 | CERS5 | 3.8 | 0.0 |
| ENSG00000278615 | C11orf98 | 3.8 | 0.0 |
| ENSG00000164120 | HPGD | 3.8 | 0.0 |
| ENSG00000090104 | RGS1 | 3.8 | 0.0 |
| ENSG00000055332 | EIF2AK2 | 3.8 | 39.8 |
| ENSG00000100342 | APOL1 | 3.8 | 25.2 |
| ENSG00000162772 | ATF3 | 3.8 | 20.1 |
| ENSG00000204103 | MAFB | 3.8 | 14.2 |
| ENSG00000121897 | LIAS | 3.8 | 5.0 |
| ENSG00000247271 | ZBED5-AS1 | 3.8 | 1.2 |
| ENSG00000197385 | ZNF860 | 3.7 | 4.0 |
| ENSG00000062598 | ELMO2 | 3.7 | 0.0 |
| ENSG00000277586 | NEFL | 3.7 | 0.0 |
| ENSG00000119139 | TJP2 | 3.7 | 10.0 |
| ENSG00000124067 | SLC12A4 | 3.7 | 0.0 |
| ENSG00000189013 | KIR2DL4 | 3.7 | 0.0 |
| ENSG00000175470 | PPP2R2D | 3.7 | 0.0 |
| ENSG00000125648 | SLC25A23 | 3.7 | 0.0 |
| ENSG00000118513 | MYB | 3.7 | 0.0 |
| ENSG00000006125 | AP2B1 | 3.7 | 0.0 |
| ENSG00000128487 | SPECC1 | 3.7 | 2.1 |
| ENSG00000197122 | SRC | 3.7 | 1.8 |
| ENSG00000059378 | PARP12 | 3.7 | 1.1 |
| ENSG00000110324 | IL10RA | 3.7 | 0.0 |
| ENSG00000148688 | RPP30 | 3.7 | 0.0 |
| ENSG00000101079 | NDRG3 | 3.7 | 0.0 |
| ENSG00000155363 | MOV10 | 3.7 | 0.0 |
| ENSG00000176393 | RNPEP | 3.7 | 0.0 |
| ENSG00000286942 | ENSG00000286942 | 3.7 | 0.0 |
| ENSG00000232677 | LINC00665 | 3.6 | 21.6 |
| ENSG00000132256 | TRIM5 | 3.6 | 11.0 |
| ENSG00000285756 | ENSG00000285756 | 3.6 | 19.5 |
| ENSG00000204856 | FAM216A | 3.6 | 0.0 |
| ENSG00000203747 | FCGR3A | 3.6 | 12.5 |
| ENSG00000147316 | MCPH1 | 3.6 | 6.8 |
| ENSG00000134748 | PRPF38A | 3.6 | 0.0 |
| ENSG00000136930 | PSMB7 | 3.6 | 0.0 |
| ENSG00000146282 | RARS2 | 3.6 | 0.0 |
| ENSG00000163872 | YEATS2 | 3.6 | 0.0 |
| ENSG00000224505 | HEXIM2-AS1 | 3.6 | 0.0 |
| ENSG00000160932 | LY6E | 3.6 | 5.7 |
| ENSG00000144655 | CSRNP1 | 3.6 | 5.6 |
| ENSG00000135392 | DNAJC14 | 3.6 | 0.0 |
| ENSG00000162390 | ACOT11 | 3.6 | 0.0 |
| ENSG00000100813 | ACIN1 | 3.6 | 0.0 |
| ENSG00000134321 | RSAD2 | 3.6 | 30.2 |
| ENSG00000185745 | IFIT1 | 3.6 | 19.6 |
| ENSG00000100441 | KHNYN | 3.6 | 4.4 |
| ENSG00000137500 | CCDC90B | 3.6 | 0.0 |
| ENSG00000109906 | ZBTB16 | 3.6 | 0.0 |
| ENSG00000188157 | AGRN | 3.6 | 0.0 |
| ENSG00000206561 | COLQ | 3.5 | 25.4 |
| ENSG00000261052 | SULT1A3 | 3.5 | 0.0 |
| ENSG00000126953 | TIMM8A | 3.5 | 31.7 |
| ENSG00000235106 | BRD3OS | 3.5 | 6.9 |
| ENSG00000186666 | BCDIN3D | 3.5 | 1.8 |
| ENSG00000143367 | TUFT1 | 3.5 | 0.0 |
| ENSG00000160145 | KALRN | 3.5 | 0.0 |
| ENSG00000010030 | ETV7 | 3.5 | 17.3 |
| ENSG00000138646 | HERC5 | 3.5 | 15.2 |
| ENSG00000154945 | ANKRD40 | 3.5 | 7.3 |
| ENSG00000088827 | SIGLEC1 | 3.5 | 6.2 |
| ENSG00000147677 | EIF3H | 3.5 | 1.2 |
| ENSG00000149534 | MS4A2 | 3.4 | 2.9 |
| ENSG00000140320 | BAHD1 | 3.4 | 0.0 |
| ENSG00000124942 | AHNAK | 3.4 | 0.0 |
| ENSG00000139055 | ERP27 | 3.4 | 0.0 |
| ENSG00000155115 | GTF3C6 | 3.4 | 0.0 |
| ENSG00000163170 | BOLA3 | 3.4 | 0.0 |
| ENSG00000066027 | PPP2R5A | 3.4 | 9.6 |
| ENSG00000107263 | RAPGEF1 | 3.4 | 0.0 |
| ENSG00000137274 | BPHL | 3.4 | 10.5 |
| ENSG00000044459 | CNTLN | 3.4 | 0.0 |
| ENSG00000198663 | C6orf89 | 3.4 | 0.0 |
| ENSG00000009335 | UBE3C | 3.4 | 0.0 |
| ENSG00000119917 | IFIT3 | 3.4 | 31.2 |
| ENSG00000183647 | ZNF530 | 3.4 | 28.3 |
| ENSG00000176124 | DLEU1 | 3.4 | 18.1 |
| ENSG00000175970 | UNC119B | 3.4 | 0.0 |
| ENSG00000173141 | MRPL57 | 3.4 | 0.0 |
| ENSG00000132286 | TIMM10B | 3.4 | 11.0 |
| ENSG00000243147 | MRPL33 | 3.4 | 0.0 |
| ENSG00000099282 | TSPAN15 | 3.4 | 26.7 |
| ENSG00000111424 | VDR | 3.3 | 17.7 |
| ENSG00000162366 | PDZK1IP1 | 3.3 | 0.0 |
| ENSG00000012124 | CD22 | 3.3 | 40.0 |
| ENSG00000184900 | SUMO3 | 3.3 | 0.0 |
| ENSG00000278784 | ENSG00000278784 | 3.3 | 0.0 |
| ENSG00000257815 | PRANCR | 3.3 | 0.0 |
| ENSG00000185730 | ZNF696 | 3.3 | 9.7 |
| ENSG00000173064 | HECTD4 | 3.3 | 0.0 |
| ENSG00000124256 | ZBP1 | 3.3 | 48.6 |
| ENSG00000023171 | GRAMD1B | 3.3 | 41.2 |
| ENSG00000149582 | TMEM25 | 3.3 | 12.1 |
| ENSG00000151806 | GUF1 | 3.3 | 1.2 |
| ENSG00000134247 | PTGFRN | 3.3 | 0.0 |
| ENSG00000213088 | ACKR1 | 3.3 | 0.0 |
| ENSG00000164062 | APEH | 3.2 | 0.0 |
| ENSG00000133661 | SFTPD | 3.2 | 0.0 |
| ENSG00000147912 | FBXO10 | 3.2 | 0.0 |
| ENSG00000125637 | PSD4 | 3.2 | 0.0 |
| ENSG00000123444 | KBTBD4 | 3.2 | 0.0 |
| ENSG00000091164 | TXNL1 | 3.2 | 0.0 |
| ENSG00000120437 | ACAT2 | 3.2 | 0.0 |
| ENSG00000168918 | INPP5D | 3.2 | 0.0 |
| ENSG00000167323 | STIM1 | 3.2 | 0.0 |
| ENSG00000234353 | ENSG00000234353 | 3.2 | 0.0 |
| ENSG00000140950 | MEAK7 | 3.2 | 0.0 |
| ENSG00000198019 | FCGR1BP | 3.2 | 42.5 |
| ENSG00000163221 | S100A12 | 3.2 | 31.3 |
| ENSG00000289963 | ENSG00000289963 | 3.2 | 33.8 |
| ENSG00000124243 | BCAS4 | 3.2 | 11.9 |
| ENSG00000274512 | TBC1D3L | 3.2 | 0.0 |
| ENSG00000196586 | MYO6 | 3.2 | 0.0 |
| ENSG00000089127 | OAS1 | 3.2 | 8.3 |
| ENSG00000172840 | PDP2 | 3.2 | 2.1 |
| ENSG00000134882 | UBAC2 | 3.1 | 0.0 |
| ENSG00000242732 | RTL5 | 3.1 | 0.0 |
| ENSG00000083454 | P2RX5 | 3.1 | 18.9 |
| ENSG00000196313 | POM121 | 3.1 | 1.4 |
| ENSG00000169252 | ADRB2 | 3.1 | 0.0 |
| ENSG00000110536 | PTPMT1 | 3.1 | 0.0 |
| ENSG00000154719 | MRPL39 | 3.1 | 0.0 |
| ENSG00000101082 | SLA2 | 3.1 | 0.0 |
| ENSG00000113552 | GNPDA1 | 3.1 | 0.0 |
| ENSG00000126003 | PLAGL2 | 3.1 | 0.0 |
| ENSG00000160783 | PMF1 | 3.1 | 0.0 |
| ENSG00000144152 | FBLN7 | 3.1 | 14.0 |
| ENSG00000225938 | S1PR1-DT | 3.1 | 0.0 |
| ENSG00000229961 | ENSG00000229961 | 3.1 | 36.7 |
| ENSG00000134146 | DPH6 | 3.1 | 4.1 |
| ENSG00000136371 | MTHFS | 3.1 | 1.4 |
| ENSG00000100373 | UPK3A | 3.1 | 1.2 |
| ENSG00000158578 | ALAS2 | 3.0 | 80.7 |
| ENSG00000130066 | SAT1 | 3.0 | 29.1 |
| ENSG00000158417 | EIF5B | 3.0 | 28.0 |
| ENSG00000128581 | IFT22 | 3.0 | 0.0 |
| ENSG00000142751 | GPN2 | 3.0 | 5.4 |
| ENSG00000205758 | CRYZL1 | 3.0 | 4.6 |
| ENSG00000001167 | NFYA | 3.0 | 0.0 |
| ENSG00000104388 | RAB2A | 3.0 | 0.0 |
| ENSG00000163947 | ARHGEF3 | 3.0 | 0.0 |
| ENSG00000144043 | TEX261 | 3.0 | 0.0 |
| ENSG00000164087 | POC1A | 3.0 | 0.0 |
| ENSG00000169057 | MECP2 | 3.0 | 0.0 |
| ENSG00000169188 | APEX2 | 3.0 | 0.0 |
| ENSG00000234127 | TRIM26 | 3.0 | 0.0 |
| ENSG00000175782 | SLC35E3 | 3.0 | 0.0 |
| ENSG00000168958 | MFF | 3.0 | 0.0 |
| ENSG00000118690 | ARMC2 | 3.0 | 0.0 |
| ENSG00000186074 | CD300LF | 2.9 | 19.9 |
| ENSG00000187608 | ISG15 | 2.9 | 13.5 |
| ENSG00000006757 | PNPLA4 | 2.9 | 0.0 |
| ENSG00000189367 | KIAA0408 | 2.9 | 0.0 |
| ENSG00000289007 | ENSG00000289007 | 2.9 | 0.0 |
| ENSG00000163069 | SGCB | 2.9 | 0.0 |
| ENSG00000171723 | GPHN | 2.9 | 0.0 |
| ENSG00000226688 | ENTPD1-AS1 | 2.9 | 0.0 |
| ENSG00000253719 | ATXN7L3B | 2.9 | 25.1 |
| ENSG00000179085 | DPM3 | 2.9 | 0.0 |
| ENSG00000089486 | CDIP1 | 2.9 | 0.0 |
| ENSG00000169020 | ATP5ME | 2.9 | 0.0 |
| ENSG00000169895 | SYAP1 | 2.9 | 0.0 |
| ENSG00000262587 | ENSG00000262587 | 2.9 | 21.8 |
| ENSG00000221869 | CEBPD | 2.9 | 0.0 |
| ENSG00000154122 | ANKH | 2.9 | 0.0 |
| ENSG00000185215 | TNFAIP2 | 2.9 | 0.0 |
| ENSG00000013523 | ANGEL1 | 2.8 | 8.1 |
| ENSG00000105426 | PTPRS | 2.8 | 43.7 |
| ENSG00000196507 | TCEAL3 | 2.8 | 23.3 |
| ENSG00000234327 | ZNF232-AS1 | 2.8 | 2.7 |
| ENSG00000233396 | LINC01719 | 2.8 | 0.0 |
| ENSG00000130589 | HELZ2 | 2.8 | 21.7 |
| ENSG00000158457 | TSPAN33 | 2.8 | 4.7 |
| ENSG00000166136 | NDUFB8 | 2.8 | 0.0 |
| ENSG00000148339 | SLC25A25 | 2.8 | 7.2 |
| ENSG00000228638 | FCF1P2 | 2.8 | 0.0 |
| ENSG00000100225 | FBXO7 | 2.8 | 10.6 |
| ENSG00000170873 | MTSS1 | 2.8 | 3.0 |
| ENSG00000148832 | PAOX | 2.8 | 0.0 |
| ENSG00000163755 | HPS3 | 2.8 | 0.0 |
| ENSG00000237424 | FOXD2-AS1 | 2.7 | 0.0 |
| ENSG00000196092 | PAX5 | 2.7 | 21.1 |
| ENSG00000261644 | CYLD-AS1 | 2.7 | 8.8 |
| ENSG00000273247 | RAB33B-AS1 | 2.7 | 7.1 |
| ENSG00000231528 | FAM225A | 2.7 | 0.0 |
| ENSG00000168228 | ZCCHC4 | 2.7 | 0.0 |
| ENSG00000135919 | SERPINE2 | 2.7 | 17.9 |
| ENSG00000100226 | GTPBP1 | 2.7 | 4.0 |
| ENSG00000288757 | ENSG00000288757 | 2.7 | 1.4 |
| ENSG00000215375 | MYL5 | 2.7 | 1.1 |
| ENSG00000278195 | SSTR3 | 2.7 | 1.1 |
| ENSG00000162298 | SYVN1 | 2.7 | 0.0 |
| ENSG00000115355 | CCDC88A | 2.7 | 0.0 |
| ENSG00000182552 | RWDD4 | 2.7 | 0.0 |
| ENSG00000137878 | GCOM1 | 2.6 | 2.4 |
| ENSG00000142687 | KIAA0319L | 2.6 | 31.3 |
| ENSG00000006534 | ALDH3B1 | 2.6 | 11.3 |
| ENSG00000196684 | HSH2D | 2.6 | 1.2 |
| ENSG00000116604 | MEF2D | 2.6 | 0.0 |
| ENSG00000218018 | RBM38-AS1 | 2.6 | 0.0 |
| ENSG00000175857 | GAPT | 2.6 | 0.0 |
| ENSG00000186204 | CYP4F12 | 2.6 | 26.0 |
| ENSG00000196517 | SLC6A9 | 2.6 | 26.5 |
| ENSG00000128886 | ELL3 | 2.6 | 17.9 |
| ENSG00000261824 | LINC00662 | 2.6 | 0.0 |
| ENSG00000169429 | CXCL8 | 2.5 | 2.5 |
| ENSG00000123374 | CDK2 | 2.5 | 1.2 |
| ENSG00000014914 | MTMR11 | 2.5 | 0.0 |
| ENSG00000214063 | TSPAN4 | 2.5 | 0.0 |
| ENSG00000225101 | OR52K3P | 2.5 | 42.8 |
| ENSG00000126709 | IFI6 | 2.5 | 20.9 |
| ENSG00000100918 | REC8 | 2.5 | 3.2 |
| ENSG00000106351 | AGFG2 | 2.5 | 0.0 |
| ENSG00000263465 | SRSF8 | 2.5 | 0.0 |
| ENSG00000108771 | DHX58 | 2.5 | 0.0 |
| ENSG00000197081 | IGF2R | 2.4 | 0.0 |
| ENSG00000124226 | RNF114 | 2.4 | 0.0 |
| ENSG00000145016 | RUBCN | 2.4 | 0.0 |
| ENSG00000134222 | PSRC1 | 2.4 | 0.0 |
| ENSG00000108559 | NUP88 | 2.4 | 15.4 |
| ENSG00000185905 | C16orf54 | 2.4 | 11.2 |
| ENSG00000068400 | GRIPAP1 | 2.4 | 11.0 |
| ENSG00000187764 | SEMA4D | 2.4 | 10.8 |
| ENSG00000144401 | METTL21A | 2.4 | 10.2 |
| ENSG00000168421 | RHOH | 2.4 | 6.8 |
| ENSG00000142784 | WDTC1 | 2.4 | 6.8 |
| ENSG00000136518 | ACTL6A | 2.4 | 6.0 |
| ENSG00000008130 | NADK | 2.4 | 5.2 |
| ENSG00000011260 | UTP18 | 2.4 | 2.4 |
| ENSG00000167984 | NLRC3 | 2.4 | 1.2 |
| ENSG00000095380 | NANS | 2.4 | 1.2 |
| ENSG00000110046 | ATG2A | 2.4 | 0.0 |
| ENSG00000105819 | PMPCB | 2.4 | 0.0 |
| ENSG00000107021 | TBC1D13 | 2.4 | 0.0 |
| ENSG00000120533 | ENY2 | 2.4 | 0.0 |
| ENSG00000145050 | MANF | 2.4 | 0.0 |
| ENSG00000196743 | GM2A | 2.4 | 0.0 |
| ENSG00000061794 | MRPS35 | 2.4 | 0.0 |
| ENSG00000111725 | PRKAB1 | 2.4 | 0.0 |
| ENSG00000134375 | TIMM17A | 2.4 | 0.0 |
| ENSG00000144034 | TPRKB | 2.4 | 0.0 |
| ENSG00000160014 | CALM3 | 2.4 | 0.0 |
| ENSG00000166997 | CNPY4 | 2.4 | 0.0 |
| ENSG00000172273 | HINFP | 2.4 | 0.0 |
| ENSG00000131018 | SYNE1 | 2.4 | 0.0 |
| ENSG00000081913 | PHLPP1 | 2.4 | 0.0 |
| ENSG00000151500 | THYN1 | 2.3 | 0.0 |
| ENSG00000124787 | RPP40 | 2.3 | 0.0 |
| ENSG00000167711 | SERPINF2 | 2.3 | 0.0 |
| ENSG00000160710 | ADAR | 2.3 | 40.2 |
| ENSG00000173334 | TRIB1 | 2.3 | 40.2 |
| ENSG00000105926 | PALS2 | 2.3 | 28.6 |
| ENSG00000112343 | TRIM38 | 2.3 | 13.4 |
| ENSG00000135423 | GLS2 | 2.3 | 3.5 |
| ENSG00000108219 | TSPAN14 | 2.3 | 2.4 |
| ENSG00000180329 | CCDC43 | 2.3 | 0.0 |
| ENSG00000138642 | HERC6 | 2.3 | 0.0 |
| ENSG00000237248 | LINC00987 | 2.3 | 0.0 |
| ENSG00000257122 | RRN3P3 | 2.3 | 0.0 |
| ENSG00000168268 | NT5DC2 | 2.3 | 0.0 |
| ENSG00000022840 | RNF10 | 2.3 | 52.8 |
| ENSG00000134827 | TCN1 | 2.3 | 9.6 |
| ENSG00000115998 | C2orf42 | 2.3 | 1.2 |
| ENSG00000174529 | TMEM81 | 2.3 | 0.0 |
| ENSG00000288823 | ENSG00000288823 | 2.3 | 0.0 |
| ENSG00000163840 | DTX3L | 2.2 | 35.8 |
| ENSG00000075142 | SRI | 2.2 | 5.4 |
| ENSG00000238083 | LRRC37A2 | 2.2 | 1.3 |
| ENSG00000198756 | COLGALT2 | 2.2 | 0.0 |
| ENSG00000166886 | NAB2 | 2.2 | 0.0 |
| ENSG00000272760 | ENSG00000272760 | 2.2 | 0.0 |
| ENSG00000172965 | MIR4435-2HG | 2.1 | 27.3 |
| ENSG00000284194 | SCO2 | 2.1 | 16.5 |
| ENSG00000108679 | LGALS3BP | 2.1 | 0.0 |
| ENSG00000164047 | CAMP | 2.1 | 0.0 |
| ENSG00000170476 | MZB1 | 2.1 | 0.0 |
| ENSG00000272941 | ENSG00000272941 | 2.1 | 63.6 |
| ENSG00000162645 | GBP2 | 2.1 | 52.4 |
| ENSG00000138496 | PARP9 | 2.1 | 49.9 |
| ENSG00000163565 | IFI16 | 2.1 | 35.3 |
| ENSG00000177409 | SAMD9L | 2.1 | 30.9 |
| ENSG00000067066 | SP100 | 2.1 | 30.2 |
| ENSG00000185880 | TRIM69 | 2.1 | 27.7 |
| ENSG00000102034 | ELF4 | 2.1 | 26.1 |
| ENSG00000002549 | LAP3 | 2.1 | 21.0 |
| ENSG00000108424 | KPNB1 | 2.1 | 17.6 |
| ENSG00000135677 | GNS | 2.1 | 14.3 |
| ENSG00000136250 | AOAH | 2.1 | 13.3 |
| ENSG00000140455 | USP3 | 2.1 | 12.4 |
| ENSG00000082397 | EPB41L3 | 2.1 | 10.9 |
| ENSG00000101019 | UQCC1 | 2.1 | 5.0 |
| ENSG00000197746 | PSAP | 2.1 | 2.8 |
| ENSG00000136826 | KLF4 | 2.1 | 2.7 |
| ENSG00000241106 | HLA-DOB | 2.1 | 2.6 |
| ENSG00000129084 | PSMA1 | 2.1 | 0.0 |
| ENSG00000120963 | ZNF706 | 2.1 | 0.0 |
| ENSG00000129757 | CDKN1C | 2.1 | 0.0 |
| ENSG00000079263 | SP140 | 2.1 | 0.0 |
| ENSG00000102699 | PARP4 | 2.1 | 0.0 |
| ENSG00000182885 | ADGRG3 | 2.1 | 29.7 |
| ENSG00000108405 | P2RX1 | 2.1 | 22.9 |
| ENSG00000272182 | ENSG00000272182 | 2.1 | 4.9 |
| ENSG00000273344 | PAXIP1-DT | 2.1 | 21.7 |
| ENSG00000115318 | LOXL3 | 2.1 | 3.5 |
| ENSG00000180479 | ZNF571 | 2.1 | 3.1 |
| ENSG00000244879 | GABPB1-AS1 | 2.1 | 0.0 |
| ENSG00000196141 | SPATS2L | 2.0 | 8.6 |
| ENSG00000178295 | GEN1 | 2.0 | 0.0 |
| ENSG00000168062 | BATF2 | 2.0 | 53.5 |
| ENSG00000132109 | TRIM21 | 2.0 | 49.6 |
| ENSG00000135148 | TRAFD1 | 2.0 | 48.3 |
| ENSG00000156587 | UBE2L6 | 2.0 | 36.7 |
| ENSG00000115415 | STAT1 | 2.0 | 34.3 |
| ENSG00000132530 | XAF1 | 2.0 | 22.7 |
| ENSG00000139083 | ETV6 | 2.0 | 19.8 |
| ENSG00000099940 | SNAP29 | 2.0 | 0.0 |
| ENSG00000128335 | APOL2 | 1.9 | 49.1 |
| ENSG00000213928 | IRF9 | 1.9 | 44.6 |
| ENSG00000125347 | IRF1 | 1.9 | 40.8 |
| ENSG00000132694 | ARHGEF11 | 1.9 | 19.5 |
| ENSG00000149547 | EI24 | 1.9 | 1.2 |
| ENSG00000181894 | ZNF329 | 1.9 | 47.4 |
| ENSG00000157734 | SNX22 | 1.9 | 45.7 |
| ENSG00000234284 | ZNF879 | 1.9 | 26.9 |
| ENSG00000132185 | FCRLA | 1.8 | 30.6 |
| ENSG00000188313 | PLSCR1 | 1.8 | 51.8 |
| ENSG00000196116 | TDRD7 | 1.8 | 25.1 |
| ENSG00000033327 | GAB2 | 1.8 | 20.4 |
| ENSG00000135926 | TMBIM1 | 1.8 | 18.4 |
| ENSG00000257923 | CUX1 | 1.8 | 17.0 |
| ENSG00000136261 | BZW2 | 1.8 | 16.1 |
| ENSG00000139651 | ZNF740 | 1.8 | 26.3 |
| ENSG00000113273 | ARSB | 1.8 | 36.7 |
| ENSG00000225151 | GOLGA2P7 | 1.7 | 34.9 |
| ENSG00000156398 | SFXN2 | 1.7 | 24.1 |
| ENSG00000228649 | SNHG26 | 1.7 | 4.6 |
| ENSG00000198873 | GRK5 | 1.7 | 0.0 |
| ENSG00000247982 | LINC00926 | 1.6 | 43.5 |
| ENSG00000197415 | VEPH1 | 1.6 | 28.1 |
| ENSG00000183161 | FANCF | 1.5 | 20.6 |
| ENSG00000079257 | LXN | 1.5 | 0.0 |
| ENSG00000224177 | LINC00570 | 1.5 | 22.9 |
| ENSG00000284820 | ENSG00000284820 | 1.5 | 0.0 |
| ENSG00000158158 | CNNM4 | 1.5 | 4.0 |
| ENSG00000137101 | CD72 | 1.5 | 38.5 |
| ENSG00000255197 | ENSG00000255197 | 1.4 | 34.3 |
| ENSG00000221963 | APOL6 | 1.4 | 26.1 |
| ENSG00000117643 | MAN1C1 | 1.4 | 19.4 |
| ENSG00000151882 | CCL28 | 1.4 | 32.8 |
| ENSG00000170915 | PAQR8 | 1.4 | 31.4 |
| ENSG00000153044 | CENPH | 1.4 | 31.1 |
| ENSG00000176396 | EID2 | 1.4 | 18.3 |
| ENSG00000083817 | ZNF416 | 1.4 | 16.4 |
| ENSG00000180509 | KCNE1 | 1.4 | 15.9 |
| ENSG00000254470 | AP5B1 | 1.4 | 52.0 |
| ENSG00000213903 | LTB4R | 1.4 | 36.1 |
| ENSG00000133106 | EPSTI1 | 1.4 | 33.4 |
| ENSG00000134326 | CMPK2 | 1.4 | 21.9 |
| ENSG00000144711 | IQSEC1 | 1.4 | 20.8 |
| ENSG00000185869 | ZNF829 | 1.4 | 14.4 |
| ENSG00000120875 | DUSP4 | 1.4 | 0.0 |
| ENSG00000146072 | TNFRSF21 | 1.3 | 24.3 |
| ENSG00000168081 | PNOC | 1.3 | 20.5 |
| ENSG00000139832 | RAB20 | 1.3 | 19.7 |
| ENSG00000111796 | KLRB1 | 1.3 | 23.3 |
| ENSG00000205639 | MFSD2B | 1.3 | 21.4 |
| ENSG00000268027 | ENSG00000268027 | 1.2 | 17.9 |
| ENSG00000240225 | ZNF542P | 1.2 | 34.0 |
| ENSG00000279320 | ENSG00000279320 | 1.2 | 32.7 |
| ENSG00000165209 | STRBP | 1.2 | 23.1 |
| ENSG00000079385 | CEACAM1 | 1.2 | 58.4 |
| ENSG00000011422 | PLAUR | 1.2 | 55.2 |
| ENSG00000161921 | CXCL16 | 1.2 | 49.6 |
| ENSG00000163932 | PRKCD | 1.2 | 45.1 |
| ENSG00000148841 | ITPRIP | 1.2 | 43.1 |
| ENSG00000135899 | SP110 | 1.2 | 43.1 |
| ENSG00000174837 | ADGRE1 | 1.2 | 37.7 |
| ENSG00000239998 | LILRA2 | 1.2 | 33.5 |
| ENSG00000167851 | CD300A | 1.2 | 33.0 |
| ENSG00000226091 | LINC00937 | 1.2 | 31.9 |
| ENSG00000146859 | TMEM140 | 1.2 | 31.1 |
| ENSG00000035862 | TIMP2 | 1.2 | 30.6 |
| ENSG00000025708 | TYMP | 1.2 | 28.9 |
| ENSG00000117298 | ECE1 | 1.2 | 28.8 |
| ENSG00000257335 | MGAM | 1.2 | 28.4 |
| ENSG00000159339 | PADI4 | 1.2 | 26.5 |
| ENSG00000244482 | LILRA6 | 1.2 | 24.8 |
| ENSG00000135821 | GLUL | 1.2 | 24.0 |
| ENSG00000172432 | GTPBP2 | 1.2 | 23.8 |
| ENSG00000125779 | PANK2 | 1.2 | 22.7 |
| ENSG00000106829 | TLE4 | 1.2 | 21.4 |
| ENSG00000143376 | SNX27 | 1.2 | 20.9 |
| ENSG00000102265 | TIMP1 | 1.2 | 20.6 |
| ENSG00000182022 | CHST15 | 1.2 | 20.1 |
| ENSG00000070614 | NDST1 | 1.2 | 19.9 |
| ENSG00000111261 | MANSC1 | 1.2 | 19.6 |
| ENSG00000178104 | PDE4DIP | 1.2 | 18.8 |
| ENSG00000181896 | ZNF101 | 1.2 | 18.4 |
| ENSG00000166889 | PATL1 | 1.2 | 18.2 |
| ENSG00000100575 | TIMM9 | 1.2 | 17.8 |
| ENSG00000162889 | MAPKAPK2 | 1.2 | 17.5 |
| ENSG00000100330 | MTMR3 | 1.2 | 17.2 |
| ENSG00000114268 | PFKFB4 | 1.2 | 17.0 |
| ENSG00000197324 | LRP10 | 1.2 | 16.6 |
| ENSG00000261218 | ENSG00000261218 | 1.2 | 16.4 |
| ENSG00000162736 | NCSTN | 1.2 | 16.2 |
| ENSG00000168028 | RPSA | 1.2 | 16.2 |
| ENSG00000198755 | RPL10A | 1.2 | 15.5 |
| ENSG00000150991 | UBC | 1.2 | 15.3 |
| ENSG00000005238 | FAM214B | 1.2 | 15.3 |
| ENSG00000140678 | ITGAX | 1.2 | 15.1 |
| ENSG00000075914 | EXOSC7 | 1.2 | 1.2 |
| ENSG00000143878 | RHOB | 1.2 | 0.0 |
| ENSG00000136143 | SUCLA2 | 1.2 | 0.0 |
| ENSG00000108010 | GLRX3 | 1.2 | 0.0 |
| ENSG00000213203 | GIMAP1 | 1.2 | 57.2 |
| ENSG00000179859 | RNF227 | 1.2 | 24.4 |
| ENSG00000121797 | CCRL2 | 1.2 | 23.1 |
| ENSG00000166278 | C2 | 1.2 | 32.6 |
| ENSG00000117228 | GBP1 | 1.1 | 42.7 |
| ENSG00000005513 | SOX8 | 1.1 | 17.6 |
| ENSG00000148459 | PDSS1 | 1.1 | 0.0 |
| ENSG00000138772 | ANXA3 | 1.1 | 32.4 |
| ENSG00000183019 | MCEMP1 | 1.1 | 25.2 |
| ENSG00000133069 | TMCC2 | 1.1 | 24.4 |
| ENSG00000068079 | IFI35 | 1.1 | 21.2 |
| ENSG00000123610 | TNFAIP6 | 1.1 | 20.3 |
| ENSG00000173369 | C1QB | 1.1 | 18.2 |
| ENSG00000079308 | TNS1 | 1.1 | 17.6 |
| ENSG00000198937 | CCDC167 | 1.1 | 0.0 |
| ENSG00000268555 | ENSG00000268555 | 1.1 | 16.8 |
| ENSG00000225411 | ENSG00000225411 | 1.1 | 16.0 |
| ENSG00000272502 | ENSG00000272502 | 1.1 | 0.0 |
| ENSG00000123095 | BHLHE41 | 1.0 | 33.8 |
| ENSG00000156265 | MAP3K7CL | 1.0 | 27.1 |
| ENSG00000181788 | SIAH2 | 0.0 | 71.8 |
| ENSG00000080561 | MID2 | 0.0 | 67.8 |
| ENSG00000184678 | H2BC21 | 0.0 | 66.3 |
| ENSG00000124635 | H2BC11 | 0.0 | 65.8 |
| ENSG00000172159 | FRMD3 | 0.0 | 64.6 |
| ENSG00000287733 | ENSG00000287733 | 0.0 | 62.2 |
| ENSG00000198734 | F5 | 0.0 | 60.7 |
| ENSG00000196867 | ZFP28 | 0.0 | 60.4 |
| ENSG00000158406 | H4C8 | 0.0 | 60.0 |
| ENSG00000198216 | CACNA1E | 0.0 | 58.9 |
| ENSG00000115828 | QPCT | 0.0 | 57.3 |
| ENSG00000174175 | SELP | 0.0 | 57.2 |
| ENSG00000183762 | KREMEN1 | 0.0 | 57.2 |
| ENSG00000154027 | AK5 | 0.0 | 56.9 |
| ENSG00000171649 | ZIK1 | 0.0 | 56.8 |
| ENSG00000188404 | SELL | 0.0 | 56.2 |
| ENSG00000068784 | SRBD1 | 0.0 | 55.9 |
| ENSG00000204580 | DDR1 | 0.0 | 55.7 |
| ENSG00000122694 | GLIPR2 | 0.0 | 55.3 |
| ENSG00000137767 | SQOR | 0.0 | 54.1 |
| ENSG00000238113 | LINC01410 | 0.0 | 54.1 |
| ENSG00000075340 | ADD2 | 0.0 | 54.0 |
| ENSG00000106537 | TSPAN13 | 0.0 | 53.3 |
| ENSG00000167434 | CA4 | 0.0 | 53.2 |
| ENSG00000110931 | CAMKK2 | 0.0 | 52.6 |
| ENSG00000112053 | SLC26A8 | 0.0 | 52.5 |
| ENSG00000167207 | NOD2 | 0.0 | 52.3 |
| ENSG00000119655 | NPC2 | 0.0 | 52.2 |
| ENSG00000159346 | ADIPOR1 | 0.0 | 52.0 |
| ENSG00000109272 | PF4V1 | 0.0 | 51.6 |
| ENSG00000161911 | TREML1 | 0.0 | 51.6 |
| ENSG00000224032 | EPB41L4A-AS1 | 0.0 | 51.6 |
| ENSG00000135318 | NT5E | 0.0 | 51.5 |
| ENSG00000113319 | RASGRF2 | 0.0 | 51.4 |
| ENSG00000109743 | BST1 | 0.0 | 51.3 |
| ENSG00000172936 | MYD88 | 0.0 | 51.2 |
| ENSG00000196954 | CASP4 | 0.0 | 51.2 |
| ENSG00000185052 | SLC24A3 | 0.0 | 51.1 |
| ENSG00000288612 | ENSG00000288612 | 0.0 | 51.1 |
| ENSG00000163754 | GYG1 | 0.0 | 50.8 |
| ENSG00000065150 | IPO5 | 0.0 | 50.6 |
| ENSG00000161955 | TNFSF13 | 0.0 | 50.4 |
| ENSG00000271303 | SRXN1 | 0.0 | 49.6 |
| ENSG00000198176 | TFDP1 | 0.0 | 49.6 |
| ENSG00000138678 | GPAT3 | 0.0 | 49.4 |
| ENSG00000132386 | SERPINF1 | 0.0 | 49.3 |
| ENSG00000159128 | IFNGR2 | 0.0 | 49.1 |
| ENSG00000171604 | CXXC5 | 0.0 | 49.0 |
| ENSG00000113140 | SPARC | 0.0 | 49.0 |
| ENSG00000111962 | UST | 0.0 | 49.0 |
| ENSG00000136827 | TOR1A | 0.0 | 49.0 |
| ENSG00000173083 | HPSE | 0.0 | 48.7 |
| ENSG00000187554 | TLR5 | 0.0 | 48.6 |
| ENSG00000109854 | HTATIP2 | 0.0 | 48.5 |
| ENSG00000180316 | PNPLA1 | 0.0 | 48.2 |
| ENSG00000183621 | ZNF438 | 0.0 | 48.2 |
| ENSG00000156381 | ANKRD9 | 0.0 | 48.0 |
| ENSG00000123130 | ACOT9 | 0.0 | 48.0 |
| ENSG00000137752 | CASP1 | 0.0 | 47.8 |
| ENSG00000059804 | SLC2A3 | 0.0 | 47.7 |
| ENSG00000005961 | ITGA2B | 0.0 | 47.6 |
| ENSG00000128203 | ASPHD2 | 0.0 | 47.4 |
| ENSG00000147457 | CHMP7 | 0.0 | 47.3 |
| ENSG00000198182 | ZNF607 | 0.0 | 47.3 |
| ENSG00000105136 | ZNF419 | 0.0 | 47.1 |
| ENSG00000188242 | PP7080 | 0.0 | 47.1 |
| ENSG00000159445 | THEM4 | 0.0 | 47.0 |
| ENSG00000137198 | GMPR | 0.0 | 46.6 |
| ENSG00000113916 | BCL6 | 0.0 | 46.5 |
| ENSG00000128512 | DOCK4 | 0.0 | 46.3 |
| ENSG00000157551 | KCNJ15 | 0.0 | 46.1 |
| ENSG00000112062 | MAPK14 | 0.0 | 46.1 |
| ENSG00000117602 | RCAN3 | 0.0 | 46.1 |
| ENSG00000068971 | PPP2R5B | 0.0 | 46.1 |
| ENSG00000290104 | ENSG00000290104 | 0.0 | 46.1 |
| ENSG00000101335 | MYL9 | 0.0 | 45.9 |
| ENSG00000162367 | TAL1 | 0.0 | 45.8 |
| ENSG00000215788 | TNFRSF25 | 0.0 | 45.6 |
| ENSG00000176871 | WSB2 | 0.0 | 45.6 |
| ENSG00000120885 | CLU | 0.0 | 45.4 |
| ENSG00000288836 | ENSG00000288836 | 0.0 | 45.4 |
| ENSG00000150337 | FCGR1A | 0.0 | 44.9 |
| ENSG00000182541 | LIMK2 | 0.0 | 44.6 |
| ENSG00000132965 | ALOX5AP | 0.0 | 44.5 |
| ENSG00000143753 | DEGS1 | 0.0 | 44.5 |
| ENSG00000157800 | SLC37A3 | 0.0 | 44.4 |
| ENSG00000169228 | RAB24 | 0.0 | 44.3 |
| ENSG00000164849 | GPR146 | 0.0 | 44.3 |
| ENSG00000137509 | PRCP | 0.0 | 44.2 |
| ENSG00000121858 | TNFSF10 | 0.0 | 43.8 |
| ENSG00000128383 | APOBEC3A | 0.0 | 43.8 |
| ENSG00000233030 | ENSG00000233030 | 0.0 | 43.8 |
| ENSG00000186431 | FCAR | 0.0 | 43.6 |
| ENSG00000143119 | CD53 | 0.0 | 43.5 |
| ENSG00000224389 | C4B | 0.0 | 43.5 |
| ENSG00000070540 | WIPI1 | 0.0 | 43.4 |
| ENSG00000145685 | LHFPL2 | 0.0 | 43.3 |
| ENSG00000096063 | SRPK1 | 0.0 | 43.3 |
| ENSG00000244734 | HBB | 0.0 | 43.3 |
| ENSG00000108984 | MAP2K6 | 0.0 | 43.3 |
| ENSG00000073792 | IGF2BP2 | 0.0 | 43.3 |
| ENSG00000116793 | PHTF1 | 0.0 | 43.2 |
| ENSG00000186918 | ZNF395 | 0.0 | 43.2 |
| ENSG00000125952 | MAX | 0.0 | 43.0 |
| ENSG00000100100 | PIK3IP1 | 0.0 | 43.0 |
| ENSG00000063046 | EIF4B | 0.0 | 42.7 |
| ENSG00000246016 | LINC01513 | 0.0 | 42.7 |
| ENSG00000204420 | MPIG6B | 0.0 | 42.7 |
| ENSG00000163191 | S100A11 | 0.0 | 42.6 |
| ENSG00000165795 | NDRG2 | 0.0 | 42.6 |
| ENSG00000101474 | APMAP | 0.0 | 42.4 |
| ENSG00000216490 | IFI30 | 0.0 | 42.2 |
| ENSG00000160785 | SLC25A44 | 0.0 | 42.1 |
| ENSG00000099204 | ABLIM1 | 0.0 | 41.9 |
| ENSG00000130830 | MPP1 | 0.0 | 41.9 |
| ENSG00000186854 | TRABD2A | 0.0 | 41.8 |
| ENSG00000268500 | SIGLEC5 | 0.0 | 41.7 |
| ENSG00000167483 | NIBAN3 | 0.0 | 41.6 |
| ENSG00000111666 | CHPT1 | 0.0 | 41.4 |
| ENSG00000244731 | C4A | 0.0 | 41.4 |
| ENSG00000140743 | CDR2 | 0.0 | 41.3 |
| ENSG00000167208 | SNX20 | 0.0 | 41.3 |
| ENSG00000138798 | EGF | 0.0 | 41.2 |
| ENSG00000256235 | SMIM3 | 0.0 | 41.2 |
| ENSG00000081059 | TCF7 | 0.0 | 41.2 |
| ENSG00000287839 | ENSG00000287839 | 0.0 | 41.2 |
| ENSG00000121413 | ZSCAN18 | 0.0 | 41.1 |
| ENSG00000161381 | PLXDC1 | 0.0 | 41.1 |
| ENSG00000178809 | TRIM73 | 0.0 | 41.1 |
| ENSG00000231607 | DLEU2 | 0.0 | 41.1 |
| ENSG00000118508 | RAB32 | 0.0 | 41.0 |
| ENSG00000081177 | EXD2 | 0.0 | 41.0 |
| ENSG00000075413 | MARK3 | 0.0 | 40.9 |
| ENSG00000136156 | ITM2B | 0.0 | 40.9 |
| ENSG00000176225 | RTTN | 0.0 | 40.8 |
| ENSG00000111644 | ACRBP | 0.0 | 40.8 |
| ENSG00000145491 | ROPN1L | 0.0 | 40.8 |
| ENSG00000270210 | ENSG00000270210 | 0.0 | 40.8 |
| ENSG00000269404 | SPIB | 0.0 | 40.6 |
| ENSG00000165030 | NFIL3 | 0.0 | 40.6 |
| ENSG00000164236 | ANKRD33B | 0.0 | 40.5 |
| ENSG00000121060 | TRIM25 | 0.0 | 40.4 |
| ENSG00000102010 | BMX | 0.0 | 40.4 |
| ENSG00000151623 | NR3C2 | 0.0 | 40.2 |
| ENSG00000162894 | FCMR | 0.0 | 40.2 |
| ENSG00000187116 | LILRA5 | 0.0 | 40.1 |
| ENSG00000124374 | PAIP2B | 0.0 | 40.0 |
| ENSG00000197208 | SLC22A4 | 0.0 | 39.9 |
| ENSG00000228956 | SATB1-AS1 | 0.0 | 39.9 |
| ENSG00000132357 | CARD6 | 0.0 | 39.9 |
| ENSG00000163568 | AIM2 | 0.0 | 39.8 |
| ENSG00000225936 | SLC18A2-AS1 | 0.0 | 39.8 |
| ENSG00000158825 | CDA | 0.0 | 39.8 |
| ENSG00000181754 | AMIGO1 | 0.0 | 39.8 |
| ENSG00000158373 | H2BC5 | 0.0 | 39.8 |
| ENSG00000278828 | H3C10 | 0.0 | 39.6 |
| ENSG00000101842 | VSIG1 | 0.0 | 39.5 |
| ENSG00000148926 | ADM | 0.0 | 39.4 |
| ENSG00000068323 | TFE3 | 0.0 | 39.2 |
| ENSG00000144218 | AFF3 | 0.0 | 39.2 |
| ENSG00000158470 | B4GALT5 | 0.0 | 39.1 |
| ENSG00000288882 | ENSG00000288882 | 0.0 | 39.0 |
| ENSG00000135334 | AKIRIN2 | 0.0 | 38.9 |
| ENSG00000128218 | VPREB3 | 0.0 | 38.9 |
| ENSG00000121933 | TMIGD3 | 0.0 | 38.9 |
| ENSG00000106624 | AEBP1 | 0.0 | 38.9 |
| ENSG00000230615 | ENSG00000230615 | 0.0 | 38.7 |
| ENSG00000187037 | GPR141 | 0.0 | 38.5 |
| ENSG00000172197 | MBOAT1 | 0.0 | 38.5 |
| ENSG00000139266 | MARCHF9 | 0.0 | 38.3 |
| ENSG00000204397 | CARD16 | 0.0 | 38.1 |
| ENSG00000170909 | OSCAR | 0.0 | 38.0 |
| ENSG00000105835 | NAMPT | 0.0 | 38.0 |
| ENSG00000064601 | CTSA | 0.0 | 37.9 |
| ENSG00000162496 | DHRS3 | 0.0 | 37.8 |
| ENSG00000177663 | IL17RA | 0.0 | 37.8 |
| ENSG00000140391 | TSPAN3 | 0.0 | 37.7 |
| ENSG00000069974 | RAB27A | 0.0 | 37.7 |
| ENSG00000186205 | MTARC1 | 0.0 | 37.6 |
| ENSG00000168056 | LTBP3 | 0.0 | 37.5 |
| ENSG00000124225 | PMEPA1 | 0.0 | 37.5 |
| ENSG00000088298 | EDEM2 | 0.0 | 37.5 |
| ENSG00000119950 | MXI1 | 0.0 | 37.4 |
| ENSG00000125485 | DDX31 | 0.0 | 37.3 |
| ENSG00000149557 | FEZ1 | 0.0 | 37.2 |
| ENSG00000169896 | ITGAM | 0.0 | 37.2 |
| ENSG00000204386 | NEU1 | 0.0 | 37.1 |
| ENSG00000133317 | LGALS12 | 0.0 | 36.9 |
| ENSG00000013583 | HEBP1 | 0.0 | 36.9 |
| ENSG00000184271 | POU6F1 | 0.0 | 36.8 |
| ENSG00000085465 | OVGP1 | 0.0 | 36.7 |
| ENSG00000005471 | ABCB4 | 0.0 | 36.7 |
| ENSG00000196793 | ZNF239 | 0.0 | 36.6 |
| ENSG00000160201 | U2AF1 | 0.0 | 36.5 |
| ENSG00000112182 | BACH2 | 0.0 | 36.5 |
| ENSG00000187650 | VMAC | 0.0 | 36.4 |
| ENSG00000112299 | VNN1 | 0.0 | 36.3 |
| ENSG00000138185 | ENTPD1 | 0.0 | 36.3 |
| ENSG00000135636 | DYSF | 0.0 | 36.2 |
| ENSG00000163162 | RNF149 | 0.0 | 36.1 |
| ENSG00000101160 | CTSZ | 0.0 | 36.1 |
| ENSG00000108861 | DUSP3 | 0.0 | 36.0 |
| ENSG00000116701 | NCF2 | 0.0 | 35.9 |
| ENSG00000185875 | THNSL1 | 0.0 | 35.8 |
| ENSG00000124762 | CDKN1A | 0.0 | 35.8 |
| ENSG00000158869 | FCER1G | 0.0 | 35.7 |
| ENSG00000139998 | RAB15 | 0.0 | 35.7 |
| ENSG00000120217 | CD274 | 0.0 | 35.7 |
| ENSG00000132274 | TRIM22 | 0.0 | 35.7 |
| ENSG00000165943 | MOAP1 | 0.0 | 35.7 |
| ENSG00000095303 | PTGS1 | 0.0 | 35.6 |
| ENSG00000124098 | FAM210B | 0.0 | 35.5 |
| ENSG00000127804 | METTL16 | 0.0 | 35.4 |
| ENSG00000249992 | TMEM158 | 0.0 | 35.4 |
| ENSG00000272914 | ENSG00000272914 | 0.0 | 35.3 |
| ENSG00000125744 | RTN2 | 0.0 | 35.3 |
| ENSG00000198106 | ENSG00000198106 | 0.0 | 35.3 |
| ENSG00000149257 | SERPINH1 | 0.0 | 35.3 |
| ENSG00000115271 | GCA | 0.0 | 35.2 |
| ENSG00000175471 | MCTP1 | 0.0 | 35.1 |
| ENSG00000102230 | PCYT1B | 0.0 | 35.1 |
| ENSG00000135404 | CD63 | 0.0 | 35.0 |
| ENSG00000166341 | DCHS1 | 0.0 | 35.0 |
| ENSG00000211459 | MT-RNR1 | 0.0 | 35.0 |
| ENSG00000114784 | EIF1B | 0.0 | 35.0 |
| ENSG00000140853 | NLRC5 | 0.0 | 34.9 |
| ENSG00000250608 | NUDT16-DT | 0.0 | 34.9 |
| ENSG00000163421 | PROK2 | 0.0 | 34.9 |
| ENSG00000164713 | BRI3 | 0.0 | 34.7 |
| ENSG00000136026 | CKAP4 | 0.0 | 34.7 |
| ENSG00000151726 | ACSL1 | 0.0 | 34.7 |
| ENSG00000225873 | C3orf86 | 0.0 | 34.6 |
| ENSG00000236144 | TMEM147-AS1 | 0.0 | 34.6 |
| ENSG00000214212 | C19orf38 | 0.0 | 34.5 |
| ENSG00000176293 | ZNF135 | 0.0 | 34.5 |
| ENSG00000134548 | SPX | 0.0 | 34.5 |
| ENSG00000162630 | B3GALT2 | 0.0 | 34.4 |
| ENSG00000198932 | GPRASP1 | 0.0 | 34.4 |
| ENSG00000152804 | HHEX | 0.0 | 34.3 |
| ENSG00000112394 | SLC16A10 | 0.0 | 34.3 |
| ENSG00000224805 | LINC00853 | 0.0 | 34.3 |
| ENSG00000060138 | YBX3 | 0.0 | 34.3 |
| ENSG00000256706 | ENSG00000256706 | 0.0 | 34.3 |
| ENSG00000107796 | ACTA2 | 0.0 | 34.2 |
| ENSG00000171051 | FPR1 | 0.0 | 34.2 |
| ENSG00000017483 | SLC38A5 | 0.0 | 34.1 |
| ENSG00000228801 | PCMTD1-DT | 0.0 | 34.1 |
| ENSG00000197283 | SYNGAP1 | 0.0 | 34.1 |
| ENSG00000135486 | HNRNPA1 | 0.0 | 33.9 |
| ENSG00000282988 | ENSG00000282988 | 0.0 | 33.9 |
| ENSG00000236901 | MIR600HG | 0.0 | 33.8 |
| ENSG00000082781 | ITGB5 | 0.0 | 33.8 |
| ENSG00000157168 | NRG1 | 0.0 | 33.8 |
| ENSG00000186407 | CD300E | 0.0 | 33.7 |
| ENSG00000134755 | DSC2 | 0.0 | 33.7 |
| ENSG00000182512 | GLRX5 | 0.0 | 33.7 |
| ENSG00000135426 | TESPA1 | 0.0 | 33.7 |
| ENSG00000205309 | NT5M | 0.0 | 33.6 |
| ENSG00000088808 | PPP1R13B | 0.0 | 33.6 |
| ENSG00000115053 | NCL | 0.0 | 33.6 |
| ENSG00000124588 | NQO2 | 0.0 | 33.5 |
| ENSG00000004939 | SLC4A1 | 0.0 | 33.3 |
| ENSG00000136689 | IL1RN | 0.0 | 33.2 |
| ENSG00000149564 | ESAM | 0.0 | 33.1 |
| ENSG00000115604 | IL18R1 | 0.0 | 33.1 |
| ENSG00000122378 | PRXL2A | 0.0 | 33.0 |
| ENSG00000118564 | FBXL5 | 0.0 | 33.0 |
| ENSG00000183696 | UPP1 | 0.0 | 32.9 |
| ENSG00000173210 | ABLIM3 | 0.0 | 32.9 |
| ENSG00000134470 | IL15RA | 0.0 | 32.9 |
| ENSG00000133985 | TTC9 | 0.0 | 32.9 |
| ENSG00000167996 | FTH1 | 0.0 | 32.8 |
| ENSG00000233461 | ENSG00000233461 | 0.0 | 32.8 |
| ENSG00000005302 | MSL3 | 0.0 | 32.8 |
| ENSG00000115956 | PLEK | 0.0 | 32.8 |
| ENSG00000196358 | NTNG2 | 0.0 | 32.7 |
| ENSG00000126262 | FFAR2 | 0.0 | 32.7 |
| ENSG00000221923 | ZNF880 | 0.0 | 32.7 |
| ENSG00000137801 | THBS1 | 0.0 | 32.6 |
| ENSG00000116044 | NFE2L2 | 0.0 | 32.6 |
| ENSG00000121064 | SCPEP1 | 0.0 | 32.5 |
| ENSG00000100985 | MMP9 | 0.0 | 32.5 |
| ENSG00000103005 | USB1 | 0.0 | 32.5 |
| ENSG00000128594 | LRRC4 | 0.0 | 32.4 |
| ENSG00000186056 | MATN1-AS1 | 0.0 | 32.4 |
| ENSG00000023318 | ERP44 | 0.0 | 32.4 |
| ENSG00000077984 | CST7 | 0.0 | 32.4 |
| ENSG00000000938 | FGR | 0.0 | 32.3 |
| ENSG00000105711 | SCN1B | 0.0 | 32.3 |
| ENSG00000103148 | NPRL3 | 0.0 | 32.2 |
| ENSG00000184500 | PROS1 | 0.0 | 32.2 |
| ENSG00000185630 | PBX1 | 0.0 | 32.2 |
| ENSG00000103037 | SETD6 | 0.0 | 32.1 |
| ENSG00000113368 | LMNB1 | 0.0 | 32.1 |
| ENSG00000069188 | SDK2 | 0.0 | 32.0 |
| ENSG00000105486 | LIG1 | 0.0 | 31.8 |
| ENSG00000087253 | LPCAT2 | 0.0 | 31.8 |
| ENSG00000161791 | FMNL3 | 0.0 | 31.8 |
| ENSG00000129351 | ILF3 | 0.0 | 31.8 |
| ENSG00000186529 | CYP4F3 | 0.0 | 31.7 |
| ENSG00000262655 | SPON1 | 0.0 | 31.7 |
| ENSG00000161835 | TAMALIN | 0.0 | 31.7 |
| ENSG00000166669 | ATF7IP2 | 0.0 | 31.7 |
| ENSG00000173114 | LRRN3 | 0.0 | 31.6 |
| ENSG00000148450 | MSRB2 | 0.0 | 31.6 |
| ENSG00000119537 | KDSR | 0.0 | 31.6 |
| ENSG00000164241 | C5orf63 | 0.0 | 31.5 |
| ENSG00000267279 | ENSG00000267279 | 0.0 | 31.5 |
| ENSG00000257218 | GATC | 0.0 | 31.4 |
| ENSG00000133056 | PIK3C2B | 0.0 | 31.4 |
| ENSG00000140545 | MFGE8 | 0.0 | 31.3 |
| ENSG00000167106 | FAM102A | 0.0 | 31.2 |
| ENSG00000155016 | CYP2U1 | 0.0 | 31.2 |
| ENSG00000204514 | ZNF814 | 0.0 | 31.1 |
| ENSG00000184613 | NELL2 | 0.0 | 31.1 |
| ENSG00000100504 | PYGL | 0.0 | 31.1 |
| ENSG00000070087 | PFN2 | 0.0 | 31.1 |
| ENSG00000122085 | MTERF4 | 0.0 | 31.1 |
| ENSG00000197296 | FITM2 | 0.0 | 31.0 |
| ENSG00000197818 | SLC9A8 | 0.0 | 31.0 |
| ENSG00000121406 | ZNF549 | 0.0 | 31.0 |
| ENSG00000095585 | BLNK | 0.0 | 30.9 |
| ENSG00000170525 | PFKFB3 | 0.0 | 30.9 |
| ENSG00000152684 | PELO | 0.0 | 30.8 |
| ENSG00000259207 | ITGB3 | 0.0 | 30.8 |
| ENSG00000165138 | ANKS6 | 0.0 | 30.8 |
| ENSG00000274536 | MIR223HG | 0.0 | 30.7 |
| ENSG00000148229 | POLE3 | 0.0 | 30.7 |
| ENSG00000163737 | PF4 | 0.0 | 30.7 |
| ENSG00000092096 | SLC22A17 | 0.0 | 30.7 |
| ENSG00000271601 | LIX1L | 0.0 | 30.7 |
| ENSG00000162747 | FCGR3B | 0.0 | 30.7 |
| ENSG00000165914 | TTC7B | 0.0 | 30.7 |
| ENSG00000118971 | CCND2 | 0.0 | 30.6 |
| ENSG00000136810 | TXN | 0.0 | 30.6 |
| ENSG00000133030 | MPRIP | 0.0 | 30.6 |
| ENSG00000197467 | COL13A1 | 0.0 | 30.6 |
| ENSG00000204424 | LY6G6F | 0.0 | 30.5 |
| ENSG00000042088 | TDP1 | 0.0 | 30.4 |
| ENSG00000101856 | PGRMC1 | 0.0 | 30.4 |
| ENSG00000073737 | DHRS9 | 0.0 | 30.3 |
| ENSG00000113269 | RNF130 | 0.0 | 30.3 |
| ENSG00000136161 | RCBTB2 | 0.0 | 30.2 |
| ENSG00000288796 | ENSG00000288796 | 0.0 | 30.2 |
| ENSG00000142089 | IFITM3 | 0.0 | 30.2 |
| ENSG00000090376 | IRAK3 | 0.0 | 30.1 |
| ENSG00000116663 | FBXO6 | 0.0 | 30.1 |
| ENSG00000072071 | ADGRL1 | 0.0 | 30.1 |
| ENSG00000175274 | TP53I11 | 0.0 | 30.0 |
| ENSG00000151687 | ANKAR | 0.0 | 30.0 |
| ENSG00000153107 | ANAPC1 | 0.0 | 29.9 |
| ENSG00000213719 | CLIC1 | 0.0 | 29.9 |
| ENSG00000142657 | PGD | 0.0 | 29.8 |
| ENSG00000101236 | RNF24 | 0.0 | 29.8 |
| ENSG00000102393 | GLA | 0.0 | 29.7 |
| ENSG00000102524 | TNFSF13B | 0.0 | 29.7 |
| ENSG00000229754 | CXCR2P1 | 0.0 | 29.6 |
| ENSG00000145979 | TBC1D7 | 0.0 | 29.6 |
| ENSG00000167664 | TMIGD2 | 0.0 | 29.6 |
| ENSG00000152454 | ZNF256 | 0.0 | 29.6 |
| ENSG00000136842 | TMOD1 | 0.0 | 29.5 |
| ENSG00000265531 | FCGR1CP | 0.0 | 29.5 |
| ENSG00000118518 | RNF146 | 0.0 | 29.5 |
| ENSG00000141698 | NT5C3B | 0.0 | 29.4 |
| ENSG00000157077 | ZFYVE9 | 0.0 | 29.4 |
| ENSG00000198189 | HSD17B11 | 0.0 | 29.3 |
| ENSG00000142528 | ZNF473 | 0.0 | 29.3 |
| ENSG00000290032 | ENSG00000290032 | 0.0 | 29.3 |
| ENSG00000034152 | MAP2K3 | 0.0 | 29.3 |
| ENSG00000151023 | ENKUR | 0.0 | 29.3 |
| ENSG00000146021 | KLHL3 | 0.0 | 29.3 |
| ENSG00000145335 | SNCA | 0.0 | 29.3 |
| ENSG00000171992 | SYNPO | 0.0 | 29.2 |
| ENSG00000243646 | IL10RB | 0.0 | 29.2 |
| ENSG00000255717 | SNHG1 | 0.0 | 29.1 |
| ENSG00000181381 | DDX60L | 0.0 | 29.1 |
| ENSG00000187912 | CLEC17A | 0.0 | 29.1 |
| ENSG00000105369 | CD79A | 0.0 | 29.1 |
| ENSG00000069275 | NUCKS1 | 0.0 | 29.0 |
| ENSG00000165490 | DDIAS | 0.0 | 29.0 |
| ENSG00000141574 | SECTM1 | 0.0 | 28.9 |
| ENSG00000196189 | SEMA4A | 0.0 | 28.9 |
| ENSG00000131778 | CHD1L | 0.0 | 28.9 |
| ENSG00000255200 | PGAM1P8 | 0.0 | 28.8 |
| ENSG00000168280 | KIF5C | 0.0 | 28.8 |
| ENSG00000262292 | GRAPLDR | 0.0 | 28.8 |
| ENSG00000143226 | FCGR2A | 0.0 | 28.7 |
| ENSG00000212719 | LINC02693 | 0.0 | 28.7 |
| ENSG00000174989 | FBXW8 | 0.0 | 28.7 |
| ENSG00000163131 | CTSS | 0.0 | 28.6 |
| ENSG00000136869 | TLR4 | 0.0 | 28.6 |
| ENSG00000169635 | HIC2 | 0.0 | 28.6 |
| ENSG00000156966 | B3GNT7 | 0.0 | 28.6 |
| ENSG00000108773 | KAT2A | 0.0 | 28.6 |
| ENSG00000088726 | TMEM40 | 0.0 | 28.4 |
| ENSG00000139178 | C1RL | 0.0 | 28.4 |
| ENSG00000197635 | DPP4 | 0.0 | 28.4 |
| ENSG00000165702 | GFI1B | 0.0 | 28.3 |
| ENSG00000103423 | DNAJA3 | 0.0 | 28.3 |
| ENSG00000093167 | LRRFIP2 | 0.0 | 28.2 |
| ENSG00000257017 | HP | 0.0 | 28.2 |
| ENSG00000150977 | RILPL2 | 0.0 | 28.2 |
| ENSG00000085511 | MAP3K4 | 0.0 | 28.2 |
| ENSG00000240065 | PSMB9 | 0.0 | 28.2 |
| ENSG00000106066 | CPVL | 0.0 | 28.2 |
| ENSG00000100558 | PLEK2 | 0.0 | 28.2 |
| ENSG00000115339 | GALNT3 | 0.0 | 28.1 |
| ENSG00000167721 | TSR1 | 0.0 | 28.0 |
| ENSG00000126368 | NR1D1 | 0.0 | 28.0 |
| ENSG00000184677 | ZBTB40 | 0.0 | 27.9 |
| ENSG00000165806 | CASP7 | 0.0 | 27.9 |
| ENSG00000235750 | KIAA0040 | 0.0 | 27.9 |
| ENSG00000062524 | LTK | 0.0 | 27.9 |
| ENSG00000021300 | PLEKHB1 | 0.0 | 27.8 |
| ENSG00000187824 | TMEM220 | 0.0 | 27.8 |
| ENSG00000120306 | CYSTM1 | 0.0 | 27.8 |
| ENSG00000146592 | CREB5 | 0.0 | 27.8 |
| ENSG00000108387 | SEPTIN4 | 0.0 | 27.7 |
| ENSG00000145911 | N4BP3 | 0.0 | 27.6 |
| ENSG00000161010 | MRNIP | 0.0 | 27.6 |
| ENSG00000174444 | RPL4 | 0.0 | 27.6 |
| ENSG00000159958 | TNFRSF13C | 0.0 | 27.6 |
| ENSG00000125538 | IL1B | 0.0 | 27.5 |
| ENSG00000134243 | SORT1 | 0.0 | 27.5 |
| ENSG00000019485 | PRDM11 | 0.0 | 27.5 |
| ENSG00000160683 | CXCR5 | 0.0 | 27.5 |
| ENSG00000143595 | AQP10 | 0.0 | 27.4 |
| ENSG00000177628 | GBA | 0.0 | 27.4 |
| ENSG00000260807 | CEROX1 | 0.0 | 27.4 |
| ENSG00000154146 | NRGN | 0.0 | 27.3 |
| ENSG00000144036 | EXOC6B | 0.0 | 27.3 |
| ENSG00000117174 | ZNHIT6 | 0.0 | 27.3 |
| ENSG00000154134 | ROBO3 | 0.0 | 27.2 |
| ENSG00000173110 | HSPA6 | 0.0 | 27.2 |
| ENSG00000174292 | TNK1 | 0.0 | 27.2 |
| ENSG00000088826 | SMOX | 0.0 | 27.2 |
| ENSG00000113648 | MACROH2A1 | 0.0 | 27.2 |
| ENSG00000188786 | MTF1 | 0.0 | 27.2 |
| ENSG00000274173 | LINC02967 | 0.0 | 27.1 |
| ENSG00000151136 | BTBD11 | 0.0 | 27.1 |
| ENSG00000147224 | PRPS1 | 0.0 | 27.0 |
| ENSG00000130713 | EXOSC2 | 0.0 | 27.0 |
| ENSG00000065923 | SLC9A7 | 0.0 | 27.0 |
| ENSG00000102409 | BEX4 | 0.0 | 27.0 |
| ENSG00000166348 | USP54 | 0.0 | 27.0 |
| ENSG00000136997 | MYC | 0.0 | 26.8 |
| ENSG00000182117 | NOP10 | 0.0 | 26.8 |
| ENSG00000007312 | CD79B | 0.0 | 26.8 |
| ENSG00000131236 | CAP1 | 0.0 | 26.8 |
| ENSG00000232021 | LEF1-AS1 | 0.0 | 26.8 |
| ENSG00000138795 | LEF1 | 0.0 | 26.7 |
| ENSG00000140479 | PCSK6 | 0.0 | 26.7 |
| ENSG00000263394 | ENSG00000263394 | 0.0 | 26.7 |
| ENSG00000049323 | LTBP1 | 0.0 | 26.7 |
| ENSG00000179840 | PIK3CD-AS1 | 0.0 | 26.7 |
| ENSG00000213762 | ZNF134 | 0.0 | 26.7 |
| ENSG00000110944 | IL23A | 0.0 | 26.6 |
| ENSG00000135503 | ACVR1B | 0.0 | 26.6 |
| ENSG00000183785 | TUBA8 | 0.0 | 26.6 |
| ENSG00000204516 | MICB | 0.0 | 26.5 |
| ENSG00000258659 | TRIM34 | 0.0 | 26.5 |
| ENSG00000101084 | RAB5IF | 0.0 | 26.5 |
| ENSG00000171860 | C3AR1 | 0.0 | 26.5 |
| ENSG00000118520 | ARG1 | 0.0 | 26.5 |
| ENSG00000203710 | CR1 | 0.0 | 26.4 |
| ENSG00000237298 | TTN-AS1 | 0.0 | 26.4 |
| ENSG00000232859 | LYRM9 | 0.0 | 26.4 |
| ENSG00000166582 | CENPV | 0.0 | 26.4 |
| ENSG00000153786 | ZDHHC7 | 0.0 | 26.4 |
| ENSG00000171552 | BCL2L1 | 0.0 | 26.4 |
| ENSG00000243273 | ENSG00000243273 | 0.0 | 26.4 |
| ENSG00000203797 | DDO | 0.0 | 26.3 |
| ENSG00000140563 | MCTP2 | 0.0 | 26.3 |
| ENSG00000196154 | S100A4 | 0.0 | 26.3 |
| ENSG00000205930 | C21orf62-AS1 | 0.0 | 26.3 |
| ENSG00000162373 | BEND5 | 0.0 | 26.2 |
| ENSG00000260943 | LINC02555 | 0.0 | 26.2 |
| ENSG00000119866 | BCL11A | 0.0 | 26.1 |
| ENSG00000163812 | ZDHHC3 | 0.0 | 26.1 |
| ENSG00000184702 | SEPTIN5 | 0.0 | 26.1 |
| ENSG00000132581 | SDF2 | 0.0 | 26.1 |
| ENSG00000280734 | LINC01232 | 0.0 | 26.0 |
| ENSG00000103569 | AQP9 | 0.0 | 26.0 |
| ENSG00000178035 | IMPDH2 | 0.0 | 25.9 |
| ENSG00000140398 | NEIL1 | 0.0 | 25.9 |
| ENSG00000245532 | NEAT1 | 0.0 | 25.9 |
| ENSG00000101558 | VAPA | 0.0 | 25.9 |
| ENSG00000163466 | ARPC2 | 0.0 | 25.8 |
| ENSG00000272079 | ENSG00000272079 | 0.0 | 25.8 |
| ENSG00000117868 | ESYT2 | 0.0 | 25.8 |
| ENSG00000102144 | PGK1 | 0.0 | 25.8 |
| ENSG00000166523 | CLEC4E | 0.0 | 25.7 |
| ENSG00000182307 | C8orf33 | 0.0 | 25.7 |
| ENSG00000144134 | RABL2A | 0.0 | 25.6 |
| ENSG00000135604 | STX11 | 0.0 | 25.6 |
| ENSG00000240057 | NEPRO-AS1 | 0.0 | 25.6 |
| ENSG00000135365 | PHF21A | 0.0 | 25.6 |
| ENSG00000238243 | OR2W3 | 0.0 | 25.6 |
| ENSG00000092036 | HAUS4 | 0.0 | 25.5 |
| ENSG00000156642 | NPTN | 0.0 | 25.5 |
| ENSG00000185201 | IFITM2 | 0.0 | 25.5 |
| ENSG00000172006 | ZNF554 | 0.0 | 25.5 |
| ENSG00000172331 | BPGM | 0.0 | 25.4 |
| ENSG00000104728 | ARHGEF10 | 0.0 | 25.4 |
| ENSG00000003402 | CFLAR | 0.0 | 25.4 |
| ENSG00000221890 | NPTXR | 0.0 | 25.3 |
| ENSG00000184014 | DENND5A | 0.0 | 25.3 |
| ENSG00000136002 | ARHGEF4 | 0.0 | 25.3 |
| ENSG00000197536 | IRF1-AS1 | 0.0 | 25.2 |
| ENSG00000245848 | CEBPA | 0.0 | 25.2 |
| ENSG00000178038 | ALS2CL | 0.0 | 25.2 |
| ENSG00000005249 | PRKAR2B | 0.0 | 25.2 |
| ENSG00000180573 | H2AC6 | 0.0 | 25.2 |
| ENSG00000173559 | NABP1 | 0.0 | 25.1 |
| ENSG00000204252 | HLA-DOA | 0.0 | 25.1 |
| ENSG00000197249 | SERPINA1 | 0.0 | 25.1 |
| ENSG00000198876 | DCAF12 | 0.0 | 25.1 |
| ENSG00000269911 | FAM226B | 0.0 | 25.0 |
| ENSG00000152894 | PTPRK | 0.0 | 25.0 |
| ENSG00000116005 | PCYOX1 | 0.0 | 25.0 |
| ENSG00000070182 | SPTB | 0.0 | 25.0 |
| ENSG00000183347 | GBP6 | 0.0 | 25.0 |
| ENSG00000163736 | PPBP | 0.0 | 25.0 |
| ENSG00000181652 | ATG9B | 0.0 | 25.0 |
| ENSG00000074660 | SCARF1 | 0.0 | 24.9 |
| ENSG00000125037 | EMC3 | 0.0 | 24.9 |
| ENSG00000166501 | PRKCB | 0.0 | 24.9 |
| ENSG00000275210 | ENSG00000275210 | 0.0 | 24.9 |
| ENSG00000289398 | ENSG00000289398 | 0.0 | 24.9 |
| ENSG00000122643 | NT5C3A | 0.0 | 24.8 |
| ENSG00000160953 | PWWP3A | 0.0 | 24.8 |
| ENSG00000006007 | GDE1 | 0.0 | 24.7 |
| ENSG00000143365 | RORC | 0.0 | 24.7 |
| ENSG00000131067 | GGT7 | 0.0 | 24.7 |
| ENSG00000143167 | GPA33 | 0.0 | 24.6 |
| ENSG00000159496 | RGL4 | 0.0 | 24.6 |
| ENSG00000155307 | SAMSN1 | 0.0 | 24.6 |
| ENSG00000127831 | VIL1 | 0.0 | 24.6 |
| ENSG00000204482 | LST1 | 0.0 | 24.6 |
| ENSG00000091106 | NLRC4 | 0.0 | 24.6 |
| ENSG00000267278 | MAP3K14-AS1 | 0.0 | 24.6 |
| ENSG00000120051 | CFAP58 | 0.0 | 24.5 |
| ENSG00000180543 | TSPYL5 | 0.0 | 24.5 |
| ENSG00000100445 | SDR39U1 | 0.0 | 24.5 |
| ENSG00000166313 | APBB1 | 0.0 | 24.2 |
| ENSG00000057935 | MTA3 | 0.0 | 24.2 |
| ENSG00000072401 | UBE2D1 | 0.0 | 24.2 |
| ENSG00000073331 | ALPK1 | 0.0 | 24.2 |
| ENSG00000231793 | DOC2GP | 0.0 | 24.2 |
| ENSG00000172264 | MACROD2 | 0.0 | 24.2 |
| ENSG00000119285 | HEATR1 | 0.0 | 24.1 |
| ENSG00000127824 | TUBA4A | 0.0 | 24.1 |
| ENSG00000117115 | PADI2 | 0.0 | 24.1 |
| ENSG00000235560 | ZNF747-DT | 0.0 | 24.1 |
| ENSG00000197256 | KANK2 | 0.0 | 24.0 |
| ENSG00000127152 | BCL11B | 0.0 | 23.9 |
| ENSG00000163041 | H3-3A | 0.0 | 23.9 |
| ENSG00000240342 | RPS2P5 | 0.0 | 23.9 |
| ENSG00000058600 | POLR3E | 0.0 | 23.9 |
| ENSG00000131368 | MRPS25 | 0.0 | 23.9 |
| ENSG00000135632 | SMYD5 | 0.0 | 23.9 |
| ENSG00000136573 | BLK | 0.0 | 23.9 |
| ENSG00000117519 | CNN3 | 0.0 | 23.9 |
| ENSG00000269837 | IPO5P1 | 0.0 | 23.9 |
| ENSG00000137103 | TMEM8B | 0.0 | 23.8 |
| ENSG00000243317 | STMP1 | 0.0 | 23.8 |
| ENSG00000115310 | RTN4 | 0.0 | 23.8 |
| ENSG00000272734 | ADIRF-AS1 | 0.0 | 23.8 |
| ENSG00000165905 | LARGE2 | 0.0 | 23.8 |
| ENSG00000114861 | FOXP1 | 0.0 | 23.7 |
| ENSG00000167565 | SERTAD3 | 0.0 | 23.7 |
| ENSG00000163625 | WDFY3 | 0.0 | 23.7 |
| ENSG00000235954 | TTC28-AS1 | 0.0 | 23.7 |
| ENSG00000035720 | STAP1 | 0.0 | 23.7 |
| ENSG00000288869 | ENSG00000288869 | 0.0 | 23.7 |
| ENSG00000142920 | AZIN2 | 0.0 | 23.7 |
| ENSG00000103647 | CORO2B | 0.0 | 23.7 |
| ENSG00000107937 | GTPBP4 | 0.0 | 23.7 |
| ENSG00000065717 | TLE2 | 0.0 | 23.6 |
| ENSG00000198959 | TGM2 | 0.0 | 23.6 |
| ENSG00000198455 | ZXDB | 0.0 | 23.6 |
| ENSG00000019991 | HGF | 0.0 | 23.6 |
| ENSG00000223705 | NSUN5P1 | 0.0 | 23.5 |
| ENSG00000170956 | CEACAM3 | 0.0 | 23.5 |
| ENSG00000100336 | APOL4 | 0.0 | 23.5 |
| ENSG00000164576 | SAP30L | 0.0 | 23.5 |
| ENSG00000170464 | DNAJC18 | 0.0 | 23.5 |
| ENSG00000168795 | ZBTB5 | 0.0 | 23.4 |
| ENSG00000131089 | ARHGEF9 | 0.0 | 23.4 |
| ENSG00000235621 | LINC00494 | 0.0 | 23.4 |
| ENSG00000146416 | AIG1 | 0.0 | 23.4 |
| ENSG00000118855 | MFSD1 | 0.0 | 23.3 |
| ENSG00000176407 | KCMF1 | 0.0 | 23.3 |
| ENSG00000143797 | MBOAT2 | 0.0 | 23.3 |
| ENSG00000225528 | ENSG00000225528 | 0.0 | 23.3 |
| ENSG00000160593 | JAML | 0.0 | 23.3 |
| ENSG00000235655 | H3P6 | 0.0 | 23.3 |
| ENSG00000104154 | SLC30A4 | 0.0 | 23.2 |
| ENSG00000183691 | NOG | 0.0 | 23.2 |
| ENSG00000140931 | CMTM3 | 0.0 | 23.2 |
| ENSG00000177989 | ODF3B | 0.0 | 23.2 |
| ENSG00000167065 | DUSP18 | 0.0 | 23.2 |
| ENSG00000145936 | KCNMB1 | 0.0 | 23.2 |
| ENSG00000054793 | ATP9A | 0.0 | 23.1 |
| ENSG00000217555 | CKLF | 0.0 | 23.1 |
| ENSG00000100721 | TCL1A | 0.0 | 23.1 |
| ENSG00000145907 | G3BP1 | 0.0 | 23.1 |
| ENSG00000206077 | ZDHHC11B | 0.0 | 23.0 |
| ENSG00000286640 | ENSG00000286640 | 0.0 | 23.0 |
| ENSG00000228340 | MIR646HG | 0.0 | 22.9 |
| ENSG00000133134 | BEX2 | 0.0 | 22.9 |
| ENSG00000136231 | IGF2BP3 | 0.0 | 22.9 |
| ENSG00000187862 | TTC24 | 0.0 | 22.8 |
| ENSG00000088832 | FKBP1A | 0.0 | 22.8 |
| ENSG00000139572 | GPR84 | 0.0 | 22.8 |
| ENSG00000197016 | ZNF470 | 0.0 | 22.8 |
| ENSG00000082146 | STRADB | 0.0 | 22.8 |
| ENSG00000167100 | SAMD14 | 0.0 | 22.8 |
| ENSG00000088053 | GP6 | 0.0 | 22.8 |
| ENSG00000165272 | AQP3 | 0.0 | 22.7 |
| ENSG00000122862 | SRGN | 0.0 | 22.7 |
| ENSG00000085063 | CD59 | 0.0 | 22.7 |
| ENSG00000080546 | SESN1 | 0.0 | 22.7 |
| ENSG00000234518 | PTGES3P1 | 0.0 | 22.6 |
| ENSG00000129355 | CDKN2D | 0.0 | 22.6 |
| ENSG00000128016 | ZFP36 | 0.0 | 22.6 |
| ENSG00000149177 | PTPRJ | 0.0 | 22.5 |
| ENSG00000138413 | IDH1 | 0.0 | 22.5 |
| ENSG00000228794 | LINC01128 | 0.0 | 22.5 |
| ENSG00000274588 | DGKK | 0.0 | 22.4 |
| ENSG00000120915 | EPHX2 | 0.0 | 22.4 |
| ENSG00000183726 | TMEM50A | 0.0 | 22.4 |
| ENSG00000123353 | ORMDL2 | 0.0 | 22.3 |
| ENSG00000154743 | TSEN2 | 0.0 | 22.3 |
| ENSG00000135473 | PAN2 | 0.0 | 22.2 |
| ENSG00000198301 | SDAD1 | 0.0 | 22.2 |
| ENSG00000170364 | SETMAR | 0.0 | 22.2 |
| ENSG00000205155 | PSENEN | 0.0 | 22.1 |
| ENSG00000168685 | IL7R | 0.0 | 22.1 |
| ENSG00000289353 | ENSG00000289353 | 0.0 | 22.1 |
| ENSG00000094916 | CBX5 | 0.0 | 22.1 |
| ENSG00000174353 | STAG3L3 | 0.0 | 22.0 |
| ENSG00000068650 | ATP11A | 0.0 | 22.0 |
| ENSG00000171843 | MLLT3 | 0.0 | 22.0 |
| ENSG00000156502 | SUPV3L1 | 0.0 | 22.0 |
| ENSG00000174669 | SLC29A2 | 0.0 | 22.0 |
| ENSG00000038945 | MSR1 | 0.0 | 22.0 |
| ENSG00000127903 | ZNF835 | 0.0 | 21.9 |
| ENSG00000165526 | RPUSD4 | 0.0 | 21.9 |
| ENSG00000275342 | PRAG1 | 0.0 | 21.9 |
| ENSG00000136048 | DRAM1 | 0.0 | 21.9 |
| ENSG00000101384 | JAG1 | 0.0 | 21.8 |
| ENSG00000224397 | PELATON | 0.0 | 21.8 |
| ENSG00000160299 | PCNT | 0.0 | 21.8 |
| ENSG00000160049 | DFFA | 0.0 | 21.8 |
| ENSG00000170832 | USP32 | 0.0 | 21.8 |
| ENSG00000103351 | CLUAP1 | 0.0 | 21.8 |
| ENSG00000100099 | HPS4 | 0.0 | 21.7 |
| ENSG00000155926 | SLA | 0.0 | 21.7 |
| ENSG00000115641 | FHL2 | 0.0 | 21.6 |
| ENSG00000120594 | PLXDC2 | 0.0 | 21.6 |
| ENSG00000108788 | MLX | 0.0 | 21.5 |
| ENSG00000170296 | GABARAP | 0.0 | 21.5 |
| ENSG00000125900 | SIRPD | 0.0 | 21.5 |
| ENSG00000241404 | EGFL8 | 0.0 | 21.5 |
| ENSG00000100473 | COCH | 0.0 | 21.5 |
| ENSG00000185885 | IFITM1 | 0.0 | 21.4 |
| ENSG00000176463 | SLCO3A1 | 0.0 | 21.4 |
| ENSG00000100612 | DHRS7 | 0.0 | 21.4 |
| ENSG00000175928 | LRRN1 | 0.0 | 21.4 |
| ENSG00000140968 | IRF8 | 0.0 | 21.4 |
| ENSG00000102245 | CD40LG | 0.0 | 21.4 |
| ENSG00000153283 | CD96 | 0.0 | 21.3 |
| ENSG00000126353 | CCR7 | 0.0 | 21.3 |
| ENSG00000104763 | ASAH1 | 0.0 | 21.3 |
| ENSG00000115590 | IL1R2 | 0.0 | 21.3 |
| ENSG00000171766 | GATM | 0.0 | 21.1 |
| ENSG00000166900 | STX3 | 0.0 | 21.1 |
| ENSG00000074966 | TXK | 0.0 | 21.1 |
| ENSG00000103550 | KNOP1 | 0.0 | 21.0 |
| ENSG00000070190 | DAPP1 | 0.0 | 21.0 |
| ENSG00000147454 | SLC25A37 | 0.0 | 21.0 |
| ENSG00000132849 | PATJ | 0.0 | 21.0 |
| ENSG00000089220 | PEBP1 | 0.0 | 21.0 |
| ENSG00000108309 | RUNDC3A | 0.0 | 21.0 |
| ENSG00000254614 | CAPN1-AS1 | 0.0 | 20.9 |
| ENSG00000183255 | PTTG1IP | 0.0 | 20.9 |
| ENSG00000165689 | ENTR1 | 0.0 | 20.9 |
| ENSG00000030110 | BAK1 | 0.0 | 20.9 |
| ENSG00000076604 | TRAF4 | 0.0 | 20.9 |
| ENSG00000270277 | ENSG00000270277 | 0.0 | 20.9 |
| ENSG00000123836 | PFKFB2 | 0.0 | 20.8 |
| ENSG00000288640 | ENSG00000288640 | 0.0 | 20.8 |
| ENSG00000258511 | LINC02295 | 0.0 | 20.8 |
| ENSG00000011105 | TSPAN9 | 0.0 | 20.8 |
| ENSG00000110934 | BIN2 | 0.0 | 20.8 |
| ENSG00000133318 | RTN3 | 0.0 | 20.8 |
| ENSG00000269821 | KCNQ1OT1 | 0.0 | 20.8 |
| ENSG00000041802 | LSG1 | 0.0 | 20.8 |
| ENSG00000082014 | SMARCD3 | 0.0 | 20.7 |
| ENSG00000047597 | XK | 0.0 | 20.7 |
| ENSG00000213246 | SUPT4H1 | 0.0 | 20.7 |
| ENSG00000099785 | MARCHF2 | 0.0 | 20.7 |
| ENSG00000180354 | MTURN | 0.0 | 20.7 |
| ENSG00000197903 | H2BC12 | 0.0 | 20.6 |
| ENSG00000123609 | NMI | 0.0 | 20.6 |
| ENSG00000166333 | ILK | 0.0 | 20.6 |
| ENSG00000079277 | MKNK1 | 0.0 | 20.5 |
| ENSG00000169239 | CA5B | 0.0 | 20.5 |
| ENSG00000101162 | TUBB1 | 0.0 | 20.5 |
| ENSG00000106133 | NSUN5P2 | 0.0 | 20.5 |
| ENSG00000140932 | CMTM2 | 0.0 | 20.5 |
| ENSG00000067992 | PDK3 | 0.0 | 20.5 |
| ENSG00000130340 | SNX9 | 0.0 | 20.5 |
| ENSG00000174125 | TLR1 | 0.0 | 20.5 |
| ENSG00000271856 | LINC01215 | 0.0 | 20.5 |
| ENSG00000205683 | DPF3 | 0.0 | 20.4 |
| ENSG00000187446 | CHP1 | 0.0 | 20.4 |
| ENSG00000264343 | NOTCH2NLA | 0.0 | 20.4 |
| ENSG00000100814 | CCNB1IP1 | 0.0 | 20.3 |
| ENSG00000104522 | GFUS | 0.0 | 20.3 |
| ENSG00000173597 | SULT1B1 | 0.0 | 20.3 |
| ENSG00000010803 | SCMH1 | 0.0 | 20.3 |
| ENSG00000132334 | PTPRE | 0.0 | 20.3 |
| ENSG00000277763 | ENSG00000277763 | 0.0 | 20.3 |
| ENSG00000225217 | HSPA7 | 0.0 | 20.3 |
| ENSG00000172967 | XKR3 | 0.0 | 20.3 |
| ENSG00000073536 | NLE1 | 0.0 | 20.3 |
| ENSG00000152213 | ARL11 | 0.0 | 20.2 |
| ENSG00000118420 | UBE3D | 0.0 | 20.2 |
| ENSG00000130684 | ZNF337 | 0.0 | 20.2 |
| ENSG00000162777 | DENND2D | 0.0 | 20.2 |
| ENSG00000130787 | HIP1R | 0.0 | 20.2 |
| ENSG00000153310 | CYRIB | 0.0 | 20.2 |
| ENSG00000100316 | RPL3 | 0.0 | 20.1 |
| ENSG00000185339 | TCN2 | 0.0 | 20.1 |
| ENSG00000204577 | LILRB3 | 0.0 | 20.0 |
| ENSG00000132475 | H3-3B | 0.0 | 20.0 |
| ENSG00000049245 | VAMP3 | 0.0 | 20.0 |
| ENSG00000089876 | DHX32 | 0.0 | 20.0 |
| ENSG00000012779 | ALOX5 | 0.0 | 20.0 |
| ENSG00000204010 | IFIT1B | 0.0 | 20.0 |
| ENSG00000166091 | CMTM5 | 0.0 | 20.0 |
| ENSG00000182487 | NCF1B | 0.0 | 19.9 |
| ENSG00000056972 | TRAF3IP2 | 0.0 | 19.9 |
| ENSG00000078699 | CBFA2T2 | 0.0 | 19.9 |
| ENSG00000018699 | TTC27 | 0.0 | 19.9 |
| ENSG00000174080 | CTSF | 0.0 | 19.8 |
| ENSG00000082512 | TRAF5 | 0.0 | 19.8 |
| ENSG00000101150 | TPD52L2 | 0.0 | 19.8 |
| ENSG00000160883 | HK3 | 0.0 | 19.8 |
| ENSG00000135378 | PRRG4 | 0.0 | 19.8 |
| ENSG00000224950 | ENSG00000224950 | 0.0 | 19.8 |
| ENSG00000118495 | PLAGL1 | 0.0 | 19.8 |
| ENSG00000099377 | HSD3B7 | 0.0 | 19.8 |
| ENSG00000141480 | ARRB2 | 0.0 | 19.7 |
| ENSG00000213347 | MXD3 | 0.0 | 19.7 |
| ENSG00000139436 | GIT2 | 0.0 | 19.6 |
| ENSG00000162551 | ALPL | 0.0 | 19.6 |
| ENSG00000289621 | ENSG00000289621 | 0.0 | 19.6 |
| ENSG00000180596 | H2BC4 | 0.0 | 19.5 |
| ENSG00000035664 | DAPK2 | 0.0 | 19.5 |
| ENSG00000290021 | ENSG00000290021 | 0.0 | 19.5 |
| ENSG00000103064 | SLC7A6 | 0.0 | 19.5 |
| ENSG00000136404 | TM6SF1 | 0.0 | 19.5 |
| ENSG00000159399 | HK2 | 0.0 | 19.5 |
| ENSG00000130348 | QRSL1 | 0.0 | 19.4 |
| ENSG00000078589 | P2RY10 | 0.0 | 19.4 |
| ENSG00000196352 | CD55 | 0.0 | 19.4 |
| ENSG00000143486 | EIF2D | 0.0 | 19.4 |
| ENSG00000181631 | P2RY13 | 0.0 | 19.4 |
| ENSG00000178562 | CD28 | 0.0 | 19.4 |
| ENSG00000141469 | SLC14A1 | 0.0 | 19.4 |
| ENSG00000272669 | ENSG00000272669 | 0.0 | 19.3 |
| ENSG00000011523 | CEP68 | 0.0 | 19.3 |
| ENSG00000105202 | FBL | 0.0 | 19.3 |
| ENSG00000185101 | ANO9 | 0.0 | 19.3 |
| ENSG00000168310 | IRF2 | 0.0 | 19.3 |
| ENSG00000124357 | NAGK | 0.0 | 19.2 |
| ENSG00000131100 | ATP6V1E1 | 0.0 | 19.2 |
| ENSG00000172594 | SMPDL3A | 0.0 | 19.2 |
| ENSG00000240505 | TNFRSF13B | 0.0 | 19.2 |
| ENSG00000189152 | GRAPL | 0.0 | 19.2 |
| ENSG00000182197 | EXT1 | 0.0 | 19.1 |
| ENSG00000124181 | PLCG1 | 0.0 | 19.1 |
| ENSG00000146094 | DOK3 | 0.0 | 19.1 |
| ENSG00000006194 | ZNF263 | 0.0 | 19.1 |
| ENSG00000169084 | DHRSX | 0.0 | 19.1 |
| ENSG00000266401 | ENSG00000266401 | 0.0 | 19.0 |
| ENSG00000273802 | H2BC8 | 0.0 | 19.0 |
| ENSG00000148110 | MFSD14B | 0.0 | 19.0 |
| ENSG00000151353 | TMEM18 | 0.0 | 19.0 |
| ENSG00000273033 | LINC02035 | 0.0 | 19.0 |
| ENSG00000187607 | ZNF286A | 0.0 | 19.0 |
| ENSG00000172071 | EIF2AK3 | 0.0 | 18.9 |
| ENSG00000184060 | ADAP2 | 0.0 | 18.9 |
| ENSG00000136819 | C9orf78 | 0.0 | 18.9 |
| ENSG00000058799 | YIPF1 | 0.0 | 18.9 |
| ENSG00000143862 | ARL8A | 0.0 | 18.8 |
| ENSG00000198858 | R3HDM4 | 0.0 | 18.8 |
| ENSG00000185475 | TMEM179B | 0.0 | 18.8 |
| ENSG00000109466 | KLHL2 | 0.0 | 18.8 |
| ENSG00000276644 | DACH1 | 0.0 | 18.8 |
| ENSG00000165006 | UBAP1 | 0.0 | 18.7 |
| ENSG00000140284 | SLC27A2 | 0.0 | 18.7 |
| ENSG00000197880 | MDS2 | 0.0 | 18.7 |
| ENSG00000130254 | SAFB2 | 0.0 | 18.7 |
| ENSG00000158828 | PINK1 | 0.0 | 18.7 |
| ENSG00000171777 | RASGRP4 | 0.0 | 18.7 |
| ENSG00000275395 | FCGBP | 0.0 | 18.6 |
| ENSG00000175063 | UBE2C | 0.0 | 18.6 |
| ENSG00000172878 | METAP1D | 0.0 | 18.6 |
| ENSG00000154309 | DISP1 | 0.0 | 18.6 |
| ENSG00000112592 | TBP | 0.0 | 18.6 |
| ENSG00000189060 | H1-0 | 0.0 | 18.5 |
| ENSG00000156206 | CFAP161 | 0.0 | 18.5 |
| ENSG00000137054 | POLR1E | 0.0 | 18.5 |
| ENSG00000283633 | ENSG00000283633 | 0.0 | 18.5 |
| ENSG00000170340 | B3GNT2 | 0.0 | 18.4 |
| ENSG00000135828 | RNASEL | 0.0 | 18.4 |
| ENSG00000204396 | VWA7 | 0.0 | 18.4 |
| ENSG00000122512 | PMS2 | 0.0 | 18.4 |
| ENSG00000258819 | LINC02289 | 0.0 | 18.4 |
| ENSG00000266236 | NARF-IT1 | 0.0 | 18.4 |
| ENSG00000198121 | LPAR1 | 0.0 | 18.4 |
| ENSG00000127951 | FGL2 | 0.0 | 18.3 |
| ENSG00000150961 | SEC24D | 0.0 | 18.3 |
| ENSG00000165678 | GHITM | 0.0 | 18.3 |
| ENSG00000269609 | C10orf95-AS1 | 0.0 | 18.2 |
| ENSG00000124731 | TREM1 | 0.0 | 18.2 |
| ENSG00000182983 | ZNF662 | 0.0 | 18.2 |
| ENSG00000166947 | EPB42 | 0.0 | 18.2 |
| ENSG00000148175 | STOM | 0.0 | 18.2 |
| ENSG00000210082 | MT-RNR2 | 0.0 | 18.1 |
| ENSG00000197006 | METTL9 | 0.0 | 18.1 |
| ENSG00000231160 | KLF3-AS1 | 0.0 | 18.1 |
| ENSG00000107742 | SPOCK2 | 0.0 | 18.1 |
| ENSG00000181481 | RNF135 | 0.0 | 18.1 |
| ENSG00000136514 | RTP4 | 0.0 | 18.1 |
| ENSG00000160208 | RRP1B | 0.0 | 18.1 |
| ENSG00000177324 | BEND2 | 0.0 | 18.1 |
| ENSG00000155275 | TRMT44 | 0.0 | 18.1 |
| ENSG00000166405 | RIC3 | 0.0 | 18.1 |
| ENSG00000196689 | TRPV1 | 0.0 | 18.0 |
| ENSG00000184465 | WDR27 | 0.0 | 18.0 |
| ENSG00000197093 | GAL3ST4 | 0.0 | 18.0 |
| ENSG00000257285 | PRMT5-DT | 0.0 | 18.0 |
| ENSG00000283602 | ENSG00000283602 | 0.0 | 18.0 |
| ENSG00000155629 | PIK3AP1 | 0.0 | 18.0 |
| ENSG00000127507 | ADGRE2 | 0.0 | 18.0 |
| ENSG00000125753 | VASP | 0.0 | 17.9 |
| ENSG00000086232 | EIF2AK1 | 0.0 | 17.9 |
| ENSG00000251002 | ENSG00000251002 | 0.0 | 17.9 |
| ENSG00000205352 | PRR13 | 0.0 | 17.9 |
| ENSG00000237943 | PRKCQ-AS1 | 0.0 | 17.9 |
| ENSG00000171049 | FPR2 | 0.0 | 17.9 |
| ENSG00000154814 | OXNAD1 | 0.0 | 17.9 |
| ENSG00000182809 | CRIP2 | 0.0 | 17.9 |
| ENSG00000123405 | NFE2 | 0.0 | 17.8 |
| ENSG00000155465 | SLC7A7 | 0.0 | 17.8 |
| ENSG00000108839 | ALOX12 | 0.0 | 17.8 |
| ENSG00000137757 | CASP5 | 0.0 | 17.8 |
| ENSG00000142961 | MOB3C | 0.0 | 17.8 |
| ENSG00000289130 | ENSG00000289130 | 0.0 | 17.8 |
| ENSG00000152380 | FAM151B | 0.0 | 17.7 |
| ENSG00000215114 | UBXN2B | 0.0 | 17.7 |
| ENSG00000145191 | EIF2B5 | 0.0 | 17.7 |
| ENSG00000158856 | DMTN | 0.0 | 17.7 |
| ENSG00000287562 | ENSG00000287562 | 0.0 | 17.7 |
| ENSG00000198690 | FAN1 | 0.0 | 17.7 |
| ENSG00000142207 | URB1 | 0.0 | 17.7 |
| ENSG00000068489 | PRR11 | 0.0 | 17.6 |
| ENSG00000170846 | ENSG00000170846 | 0.0 | 17.6 |
| ENSG00000158517 | NCF1 | 0.0 | 17.6 |
| ENSG00000076554 | TPD52 | 0.0 | 17.6 |
| ENSG00000091490 | SEL1L3 | 0.0 | 17.5 |
| ENSG00000126602 | TRAP1 | 0.0 | 17.5 |
| ENSG00000100030 | MAPK1 | 0.0 | 17.5 |
| ENSG00000138449 | SLC40A1 | 0.0 | 17.5 |
| ENSG00000113048 | MRPS27 | 0.0 | 17.5 |
| ENSG00000187621 | TCL6 | 0.0 | 17.5 |
| ENSG00000136315 | RNASE2CP | 0.0 | 17.4 |
| ENSG00000122965 | RBM19 | 0.0 | 17.4 |
| ENSG00000198478 | SH3BGRL2 | 0.0 | 17.4 |
| ENSG00000184117 | NIPSNAP1 | 0.0 | 17.4 |
| ENSG00000101546 | RBFA | 0.0 | 17.4 |
| ENSG00000272462 | LINC02980 | 0.0 | 17.3 |
| ENSG00000029534 | ANK1 | 0.0 | 17.3 |
| ENSG00000185862 | EVI2B | 0.0 | 17.3 |
| ENSG00000141447 | OSBPL1A | 0.0 | 17.3 |
| ENSG00000234741 | GAS5 | 0.0 | 17.3 |
| ENSG00000158089 | GALNT14 | 0.0 | 17.3 |
| ENSG00000105373 | NOP53 | 0.0 | 17.2 |
| ENSG00000129450 | SIGLEC9 | 0.0 | 17.2 |
| ENSG00000188042 | ARL4C | 0.0 | 17.2 |
| ENSG00000110841 | PPFIBP1 | 0.0 | 17.2 |
| ENSG00000188234 | AGAP4 | 0.0 | 17.2 |
| ENSG00000182841 | RRP7BP | 0.0 | 17.2 |
| ENSG00000076685 | NT5C2 | 0.0 | 17.1 |
| ENSG00000166949 | SMAD3 | 0.0 | 17.1 |
| ENSG00000125875 | TBC1D20 | 0.0 | 17.1 |
| ENSG00000177455 | CD19 | 0.0 | 17.1 |
| ENSG00000197956 | S100A6 | 0.0 | 17.1 |
| ENSG00000143321 | HDGF | 0.0 | 17.1 |
| ENSG00000247746 | USP51 | 0.0 | 17.1 |
| ENSG00000153162 | BMP6 | 0.0 | 17.1 |
| ENSG00000166428 | PLD4 | 0.0 | 17.1 |
| ENSG00000182670 | TTC3 | 0.0 | 17.0 |
| ENSG00000175061 | SNHG29 | 0.0 | 17.0 |
| ENSG00000087086 | FTL | 0.0 | 17.0 |
| ENSG00000112195 | TREML2 | 0.0 | 17.0 |
| ENSG00000115977 | AAK1 | 0.0 | 17.0 |
| ENSG00000137575 | SDCBP | 0.0 | 16.9 |
| ENSG00000170837 | GPR27 | 0.0 | 16.9 |
| ENSG00000184515 | BEX5 | 0.0 | 16.9 |
| ENSG00000137193 | PIM1 | 0.0 | 16.9 |
| ENSG00000118402 | ELOVL4 | 0.0 | 16.9 |
| ENSG00000257743 | MGAM2 | 0.0 | 16.9 |
| ENSG00000143207 | COP1 | 0.0 | 16.9 |
| ENSG00000184792 | OSBP2 | 0.0 | 16.9 |
| ENSG00000229124 | VIM-AS1 | 0.0 | 16.8 |
| ENSG00000133606 | MKRN1 | 0.0 | 16.8 |
| ENSG00000100823 | APEX1 | 0.0 | 16.8 |
| ENSG00000078319 | PMS2P1 | 0.0 | 16.8 |
| ENSG00000111729 | CLEC4A | 0.0 | 16.8 |
| ENSG00000198346 | ZNF813 | 0.0 | 16.8 |
| ENSG00000168297 | PXK | 0.0 | 16.8 |
| ENSG00000124102 | PI3 | 0.0 | 16.8 |
| ENSG00000177337 | DLGAP1-AS1 | 0.0 | 16.7 |
| ENSG00000135074 | ADAM19 | 0.0 | 16.7 |
| ENSG00000149970 | CNKSR2 | 0.0 | 16.7 |
| ENSG00000124788 | ATXN1 | 0.0 | 16.7 |
| ENSG00000160991 | ORAI2 | 0.0 | 16.7 |
| ENSG00000150477 | KIAA1328 | 0.0 | 16.7 |
| ENSG00000173930 | SLCO4C1 | 0.0 | 16.7 |
| ENSG00000059728 | MXD1 | 0.0 | 16.6 |
| ENSG00000143337 | TOR1AIP1 | 0.0 | 16.6 |
| ENSG00000166002 | SMCO4 | 0.0 | 16.6 |
| ENSG00000260916 | CCPG1 | 0.0 | 16.6 |
| ENSG00000288932 | ENSG00000288932 | 0.0 | 16.6 |
| ENSG00000196724 | ZNF418 | 0.0 | 16.5 |
| ENSG00000260719 | ENSG00000260719 | 0.0 | 16.5 |
| ENSG00000107815 | TWNK | 0.0 | 16.5 |
| ENSG00000213625 | LEPROT | 0.0 | 16.4 |
| ENSG00000213523 | SRA1 | 0.0 | 16.4 |
| ENSG00000173281 | PPP1R3B | 0.0 | 16.4 |
| ENSG00000254772 | EEF1G | 0.0 | 16.4 |
| ENSG00000234171 | RNASEH1-DT | 0.0 | 16.4 |
| ENSG00000187800 | PEAR1 | 0.0 | 16.4 |
| ENSG00000147274 | RBMX | 0.0 | 16.3 |
| ENSG00000135363 | LMO2 | 0.0 | 16.3 |
| ENSG00000103740 | ACSBG1 | 0.0 | 16.3 |
| ENSG00000143409 | MINDY1 | 0.0 | 16.3 |
| ENSG00000102967 | DHODH | 0.0 | 16.2 |
| ENSG00000169570 | DTWD2 | 0.0 | 16.2 |
| ENSG00000085788 | DDHD2 | 0.0 | 16.2 |
| ENSG00000169122 | FAM110B | 0.0 | 16.2 |
| ENSG00000134668 | SPOCD1 | 0.0 | 16.2 |
| ENSG00000072858 | SIDT1 | 0.0 | 16.1 |
| ENSG00000184205 | TSPYL2 | 0.0 | 16.1 |
| ENSG00000106636 | YKT6 | 0.0 | 16.1 |
| ENSG00000116991 | SIPA1L2 | 0.0 | 16.1 |
| ENSG00000147789 | ZNF7 | 0.0 | 16.0 |
| ENSG00000204219 | TCEA3 | 0.0 | 16.0 |
| ENSG00000285796 | ENSG00000285796 | 0.0 | 16.0 |
| ENSG00000189077 | TMEM120A | 0.0 | 16.0 |
| ENSG00000127920 | GNG11 | 0.0 | 16.0 |
| ENSG00000076513 | ANKRD13A | 0.0 | 15.9 |
| ENSG00000267481 | ENSG00000267481 | 0.0 | 15.9 |
| ENSG00000169826 | CSGALNACT2 | 0.0 | 15.9 |
| ENSG00000161298 | ZNF382 | 0.0 | 15.9 |
| ENSG00000174428 | GTF2IRD2B | 0.0 | 15.9 |
| ENSG00000119004 | CYP20A1 | 0.0 | 15.9 |
| ENSG00000213988 | ZNF90 | 0.0 | 15.9 |
| ENSG00000137337 | MDC1 | 0.0 | 15.8 |
| ENSG00000198453 | ZNF568 | 0.0 | 15.8 |
| ENSG00000129484 | PARP2 | 0.0 | 15.8 |
| ENSG00000026559 | KCNG1 | 0.0 | 15.8 |
| ENSG00000130956 | HABP4 | 0.0 | 15.8 |
| ENSG00000163235 | TGFA | 0.0 | 15.7 |
| ENSG00000171314 | PGAM1 | 0.0 | 15.7 |
| ENSG00000100897 | DCAF11 | 0.0 | 15.7 |
| ENSG00000154174 | TOMM70 | 0.0 | 15.6 |
| ENSG00000134463 | ECHDC3 | 0.0 | 15.6 |
| ENSG00000146376 | ARHGAP18 | 0.0 | 15.6 |
| ENSG00000166025 | AMOTL1 | 0.0 | 15.6 |
| ENSG00000144559 | TAMM41 | 0.0 | 15.6 |
| ENSG00000126249 | PDCD2L | 0.0 | 15.5 |
| ENSG00000096968 | JAK2 | 0.0 | 15.5 |
| ENSG00000151553 | FHIP2A | 0.0 | 15.5 |
| ENSG00000121316 | PLBD1 | 0.0 | 15.5 |
| ENSG00000286010 | ENSG00000286010 | 0.0 | 15.5 |
| ENSG00000132825 | PPP1R3D | 0.0 | 15.5 |
| ENSG00000129467 | ADCY4 | 0.0 | 15.5 |
| ENSG00000154016 | GRAP | 0.0 | 15.4 |
| ENSG00000270164 | LINC01480 | 0.0 | 15.4 |
| ENSG00000249115 | HAUS5 | 0.0 | 15.4 |
| ENSG00000146112 | PPP1R18 | 0.0 | 15.4 |
| ENSG00000043462 | LCP2 | 0.0 | 15.4 |
| ENSG00000196209 | SIRPB2 | 0.0 | 15.3 |
| ENSG00000197548 | ATG7 | 0.0 | 15.3 |
| ENSG00000266947 | ENSG00000266947 | 0.0 | 15.3 |
| ENSG00000127334 | DYRK2 | 0.0 | 15.3 |
| ENSG00000139132 | FGD4 | 0.0 | 15.3 |
| ENSG00000075275 | CELSR1 | 0.0 | 15.2 |
| ENSG00000065357 | DGKA | 0.0 | 15.2 |
| ENSG00000174748 | RPL15 | 0.0 | 15.2 |
| ENSG00000108846 | ABCC3 | 0.0 | 15.2 |
| ENSG00000111057 | KRT18 | 0.0 | 15.1 |
| ENSG00000105507 | CABP5 | 0.0 | 15.1 |
| ENSG00000226891 | LINC01359 | 0.0 | 15.1 |
| ENSG00000269896 | ENSG00000269896 | 0.0 | 15.1 |
| ENSG00000065615 | CYB5R4 | 0.0 | 15.1 |
| ENSG00000162881 | OXER1 | 0.0 | 15.1 |
| ENSG00000142405 | NLRP12 | 0.0 | 15.1 |
| ENSG00000151948 | GLT1D1 | 0.0 | 15.1 |
| ENSG00000198363 | ASPH | 0.0 | 15.1 |
| ENSG00000166526 | ZNF3 | 0.0 | 15.0 |
| ENSG00000244627 | TPTEP2 | 0.0 | 15.0 |
| ENSG00000119280 | C1orf198 | 0.0 | 15.0 |
| ENSG00000132300 | PTCD3 | 0.0 | 14.1 |
| ENSG00000140382 | HMG20A | 0.0 | 13.7 |
| ENSG00000185760 | KCNQ5 | 0.0 | 13.0 |
| ENSG00000186891 | TNFRSF18 | 0.0 | 12.2 |
| ENSG00000102221 | JADE3 | 0.0 | 12.0 |
| ENSG00000110079 | MS4A4A | 0.0 | 11.8 |
| ENSG00000168961 | LGALS9 | 0.0 | 11.7 |
| ENSG00000204301 | NOTCH4 | 0.0 | 9.3 |
| ENSG00000102978 | POLR2C | 0.0 | 7.5 |
| ENSG00000157837 | SPPL3 | 0.0 | 7.4 |
| ENSG00000163026 | WDCP | 0.0 | 6.4 |
| ENSG00000124207 | CSE1L | 0.0 | 6.2 |
| ENSG00000148848 | ADAM12 | 0.0 | 6.2 |
| ENSG00000134490 | TMEM241 | 0.0 | 5.9 |
| ENSG00000169981 | ZNF35 | 0.0 | 5.8 |
| ENSG00000002834 | LASP1 | 0.0 | 5.2 |
| ENSG00000008516 | MMP25 | 0.0 | 5.0 |
| ENSG00000284879 | ENSG00000284879 | 0.0 | 5.0 |
| ENSG00000115504 | EHBP1 | 0.0 | 4.6 |
| ENSG00000149474 | KAT14 | 0.0 | 4.6 |
| ENSG00000175003 | SLC22A1 | 0.0 | 3.9 |
| ENSG00000106344 | RBM28 | 0.0 | 3.8 |
| ENSG00000203739 | PRDX6-AS1 | 0.0 | 3.2 |
| ENSG00000123427 | EEF1AKMT3 | 0.0 | 3.1 |
| ENSG00000042062 | RIPOR3 | 0.0 | 3.1 |
| ENSG00000146731 | CCT6A | 0.0 | 3.0 |
| ENSG00000233024 | NPIPA9 | 0.0 | 2.4 |
| ENSG00000164048 | ZNF589 | 0.0 | 2.4 |
| ENSG00000165630 | PRPF18 | 0.0 | 2.4 |
| ENSG00000115507 | OTX1 | 0.0 | 2.3 |
| ENSG00000196550 | FAM72A | 0.0 | 2.2 |
| ENSG00000169902 | TPST1 | 0.0 | 2.1 |
| ENSG00000002726 | AOC1 | 0.0 | 2.0 |
| ENSG00000247679 | ENSG00000247679 | 0.0 | 2.0 |
| ENSG00000166343 | MSS51 | 0.0 | 1.4 |
| ENSG00000138670 | RASGEF1B | 0.0 | 1.3 |
| ENSG00000166848 | TERF2IP | 0.0 | 1.2 |
| ENSG00000127463 | EMC1 | 0.0 | 1.2 |
| ENSG00000138101 | DTNB | 0.0 | 1.2 |
| ENSG00000225205 | PDK1-AS1 | 0.0 | 1.2 |

TABLE 2

| | | AA | CRC |
|---|---|---|---|
| ensemble ID | gene symbol | score | score |
| ENSG00000128656 | CHN1 | 52.02 | 0.00 |
| ENSG00000289561 | ENSG00000289561 | 51.93 | 2.00 |
| ENSG00000113296 | THBS4 | 49.16 | 0.00 |
| ENSG00000105205 | CLC | 48.70 | 1.10 |
| ENSG00000115602 | IL1RL1 | 47.95 | 2.37 |
| ENSG00000181804 | SLC9A9 | 46.50 | 40.03 |
| ENSG00000263961 | RHEX | 46.32 | 0.00 |
| ENSG00000131203 | IDO1 | 45.50 | 9.73 |
| ENSG00000103056 | SMPD3 | 44.12 | 0.00 |
| ENSG00000273314 | ENSG00000273314 | 43.66 | 0.00 |
| ENSG00000188033 | ZNF490 | 43.29 | 0.00 |
| ENSG00000282608 | ADORA3 | 42.70 | 0.00 |
| ENSG00000169397 | RNASE3 | 42.20 | 0.00 |
| ENSG00000142408 | CACNG8 | 41.80 | 4.20 |
| ENSG00000091181 | IL5RA | 41.47 | 1.15 |
| ENSG00000184208 | C22orf46 | 40.72 | 4.25 |
| ENSG00000143653 | SCCPDH | 40.03 | 0.00 |
| ENSG00000121691 | CAT | 39.80 | 0.00 |
| ENSG00000182557 | SPNS3 | 39.35 | 0.00 |
| ENSG00000175591 | P2RY2 | 39.28 | 0.00 |
| ENSG00000103355 | PRSS33 | 38.73 | 6.19 |
| ENSG00000089091 | DZANK1 | 37.87 | 0.00 |
| ENSG00000124019 | FAM124B | 37.36 | 0.00 |
| ENSG00000235217 | TSPY26P | 36.00 | 0.00 |
| ENSG00000174944 | P2RY14 | 34.33 | 2.20 |
| ENSG00000090470 | PDCD7 | 32.96 | 12.75 |
| ENSG00000255587 | RAB44 | 32.78 | 1.15 |
| ENSG00000128596 | CCDC136 | 31.98 | 0.00 |
| ENSG00000161905 | ALOX15 | 31.27 | 0.00 |
| ENSG00000175048 | ZDHHC14 | 31.00 | 0.00 |
| ENSG00000120279 | MYCT1 | 30.62 | 0.00 |
| ENSG00000138617 | PARP16 | 30.10 | 0.00 |
| ENSG00000105366 | SIGLEC8 | 29.28 | 4.00 |
| ENSG00000232788 | ITGA6-AS1 | 29.04 | 0.00 |
| ENSG00000173166 | RAPH1 | 28.07 | 1.30 |
| ENSG00000112759 | SLC29A1 | 27.40 | 4.87 |
| ENSG00000140263 | SORD | 26.70 | 35.17 |
| ENSG00000276231 | PIK3R6 | 26.27 | 5.88 |
| ENSG00000290017 | ENSG00000290017 | 26.14 | 10.05 |
| ENSG00000186197 | EDARADD | 25.50 | 0.00 |
| ENSG00000013619 | MAMLD1 | 25.29 | 0.00 |
| ENSG00000204165 | CXorf65 | 25.23 | 1.33 |
| ENSG00000239732 | TLR9 | 25.15 | 6.35 |
| ENSG00000048162 | NOP16 | 24.82 | 3.33 |
| ENSG00000133424 | LARGE1 | 24.82 | 6.90 |
| ENSG00000135414 | GDF11 | 24.60 | 19.10 |
| ENSG00000214174 | AMZ2P1 | 24.47 | 0.00 |
| ENSG00000132718 | SYT11 | 24.43 | 1.15 |
| ENSG00000145730 | PAM | 23.47 | 0.00 |
| ENSG00000204444 | APOM | 23.40 | 0.00 |
| ENSG00000130433 | CACNG6 | 23.10 | 0.00 |
| ENSG00000125869 | LAMP5 | 23.09 | 22.10 |
| ENSG00000119673 | ACOT2 | 23.05 | 0.00 |
| ENSG00000144290 | SLC4A10 | 23.03 | 18.93 |
| ENSG00000176903 | PNMA1 | 22.63 | 0.00 |
| ENSG00000119401 | TRIM32 | 22.30 | 6.07 |
| ENSG00000233038 | PTPRN2-AS1 | 22.20 | 0.00 |
| ENSG00000029993 | HMGB3 | 22.15 | 0.00 |
| ENSG00000176083 | ZNF683 | 22.13 | 0.00 |
| ENSG00000185090 | MANEAL | 22.02 | 2.80 |
| ENSG00000011638 | LDAF1 | 21.67 | 0.00 |
| ENSG00000189068 | VSTM1 | 21.60 | 25.67 |
| ENSG00000182195 | LDOC1 | 21.58 | 0.00 |
| ENSG00000119689 | DLST | 21.03 | 0.00 |
| ENSG00000092067 | CEBPE | 21.00 | 0.00 |
| ENSG00000184154 | LRRC51 | 20.69 | 0.00 |
| ENSG00000279693 | ENSG00000279693 | 20.37 | 0.00 |
| ENSG00000184221 | OLIG1 | 20.33 | 0.00 |
| ENSG00000188282 | RUFY4 | 20.32 | 5.00 |
| ENSG00000256128 | LINC00944 | 20.23 | 0.00 |
| ENSG00000166086 | JAM3 | 20.20 | 1.02 |
| ENSG00000026950 | BTN3A1 | 20.19 | 0.00 |
| ENSG00000245904 | BTG1-DT | 20.05 | 21.18 |
| ENSG00000164663 | USP49 | 20.00 | 0.00 |
| ENSG00000123358 | NR4A1 | 19.86 | 0.00 |
| ENSG00000083844 | ZNF264 | 19.85 | 2.25 |
| ENSG00000232098 | ZNF584-DT | 19.30 | 0.00 |
| ENSG00000204472 | AIF1 | 19.29 | 5.47 |
| ENSG00000100448 | CTSG | 19.03 | 0.00 |
| ENSG00000134489 | HRH4 | 18.93 | 1.62 |
| ENSG00000020181 | ADGRA2 | 18.88 | 0.00 |
| ENSG00000196220 | SRGAP3 | 18.87 | 0.00 |
| ENSG00000083814 | ZNF671 | 18.83 | 7.60 |
| ENSG00000184857 | TMEM186 | 18.80 | 0.00 |
| ENSG00000166016 | ABTB2 | 18.77 | 0.00 |
| ENSG00000158050 | DUSP2 | 18.67 | 0.00 |
| ENSG00000150093 | ITGB1 | 18.23 | 0.00 |
| ENSG00000144115 | THNSL2 | 18.17 | 0.00 |
| ENSG00000178538 | CA8 | 18.10 | 0.00 |
| ENSG00000138031 | ADCY3 | 17.97 | 1.07 |
| ENSG00000290043 | ENSG00000290043 | 17.80 | 0.00 |
| ENSG00000169398 | PTK2 | 17.77 | 0.00 |
| ENSG00000005381 | MPO | 17.57 | 0.00 |
| ENSG00000185291 | IL3RA | 17.50 | 0.00 |
| ENSG00000152465 | NMT2 | 17.47 | 18.70 |
| ENSG00000128973 | CLN6 | 17.20 | 0.00 |
| ENSG00000112149 | CD83 | 17.03 | 9.67 |
| ENSG00000008513 | ST3GAL1 | 16.90 | 0.00 |
| ENSG00000099977 | DDT | 16.80 | 0.00 |
| ENSG00000176371 | ZSCAN2 | 16.79 | 0.00 |
| ENSG00000168404 | MLKL | 16.30 | 40.03 |
| ENSG00000288838 | ENSG00000288838 | 10.40 | 43.60 |
| ENSG00000146425 | DYNLT1 | 7.87 | 38.83 |
| ENSG00000153064 | BANK1 | 7.00 | 42.87 |
| ENSG00000153029 | MR1 | 7.00 | 34.70 |
| ENSG00000144567 | RETREG2 | 6.83 | 41.70 |
| ENSG00000116717 | GADD45A | 6.83 | 42.43 |
| ENSG00000162722 | TRIM58 | 5.73 | 53.40 |
| ENSG00000110987 | BCL7A | 5.52 | 38.56 |
| ENSG00000259162 | ENSG00000259162 | 5.45 | 42.05 |
| ENSG00000112486 | CCR6 | 5.15 | 37.45 |
| ENSG00000156738 | MS4A1 | 5.00 | 37.03 |
| ENSG00000168394 | TAP1 | 4.40 | 46.73 |
| ENSG00000136816 | TOR1B | 4.40 | 37.10 |
| ENSG00000152229 | PSTPIP2 | 4.27 | 52.17 |
| ENSG00000173193 | PARP14 | 3.97 | 35.17 |
| ENSG00000055332 | EIF2AK2 | 3.77 | 39.77 |
| ENSG00000012124 | CD22 | 3.33 | 40.03 |
| ENSG00000124256 | ZBP1 | 3.27 | 48.57 |
| ENSG00000023171 | GRAMD1B | 3.27 | 41.17 |
| ENSG00000198019 | FCGR1BP | 3.20 | 42.53 |
| ENSG00000229961 | ENSG00000229961 | 3.07 | 36.73 |
| ENSG00000158578 | ALAS2 | 3.03 | 80.73 |
| ENSG00000105426 | PTPRS | 2.83 | 43.69 |
| ENSG00000225101 | OR52K3P | 2.47 | 42.80 |
| ENSG00000160710 | ADAR | 2.30 | 40.23 |
| ENSG00000173334 | TRIB1 | 2.30 | 40.17 |
| ENSG00000022840 | RNF10 | 2.27 | 52.77 |
| ENSG00000163840 | DTX3L | 2.20 | 35.76 |
| ENSG00000272941 | ENSG00000272941 | 2.10 | 63.57 |
| ENSG00000162645 | GBP2 | 2.10 | 52.40 |
| ENSG00000138496 | PARP9 | 2.10 | 49.93 |
| ENSG00000163565 | IFI16 | 2.10 | 35.33 |
| ENSG00000168062 | BATF2 | 2.00 | 53.47 |
| ENSG00000132109 | TRIM21 | 2.00 | 49.60 |
| ENSG00000135148 | TRAFD1 | 2.00 | 48.30 |
| ENSG00000156587 | UBE2L6 | 2.00 | 36.70 |
| ENSG00000128335 | APOL2 | 1.90 | 49.10 |
| ENSG00000213928 | IRF9 | 1.90 | 44.63 |
| ENSG00000125347 | IRF1 | 1.90 | 40.80 |
| ENSG00000181894 | ZNF329 | 1.85 | 47.40 |
| ENSG00000157734 | SNX22 | 1.85 | 45.68 |
| ENSG00000188313 | PLSCR1 | 1.80 | 51.83 |
| ENSG00000113273 | ARSB | 1.75 | 36.74 |
| ENSG00000225151 | GOLGA2P7 | 1.65 | 34.91 |
| ENSG00000247982 | LINC00926 | 1.57 | 43.50 |
| ENSG00000137101 | CD72 | 1.47 | 38.50 |
| ENSG00000255197 | ENSG00000255197 | 1.43 | 34.30 |
| ENSG00000254470 | AP5B1 | 1.40 | 52.00 |
| ENSG00000213903 | LTB4R | 1.40 | 36.07 |
| ENSG00000079385 | CEACAM1 | 1.20 | 58.37 |
| ENSG00000011422 | PLAUR | 1.20 | 55.20 |
| ENSG00000161921 | CXCL16 | 1.20 | 49.60 |
| ENSG00000163932 | PRKCD | 1.20 | 45.13 |
| ENSG00000148841 | ITPRIP | 1.20 | 43.10 |
| ENSG00000135899 | SP110 | 1.20 | 43.07 |
| ENSG00000174837 | ADGRE1 | 1.20 | 37.73 |
| ENSG00000213203 | GIMAP1 | 1.20 | 57.18 |
| ENSG00000117228 | GBP1 | 1.13 | 42.73 |
| ENSG00000181788 | SIAH2 | 0.00 | 71.83 |
| ENSG00000080561 | MID2 | 0.00 | 67.79 |
| ENSG00000184678 | H2BC21 | 0.00 | 66.29 |
| ENSG00000124635 | H2BC11 | 0.00 | 65.83 |
| ENSG00000172159 | FRMD3 | 0.00 | 64.56 |
| ENSG00000287733 | ENSG00000287733 | 0.00 | 62.17 |
| ENSG00000198734 | F5 | 0.00 | 60.70 |
| ENSG00000196867 | ZFP28 | 0.00 | 60.36 |
| ENSG00000158406 | H4C8 | 0.00 | 59.99 |
| ENSG00000198216 | CACNA1E | 0.00 | 58.87 |
| ENSG00000115828 | QPCT | 0.00 | 57.33 |
| ENSG00000174175 | SELP | 0.00 | 57.23 |
| ENSG00000183762 | KREMEN1 | 0.00 | 57.20 |
| ENSG00000154027 | AK5 | 0.00 | 56.88 |
| ENSG00000171649 | ZIK1 | 0.00 | 56.83 |
| ENSG00000188404 | SELL | 0.00 | 56.20 |
| ENSG00000068784 | SRBD1 | 0.00 | 55.90 |
| ENSG00000204580 | DDR1 | 0.00 | 55.65 |
| ENSG00000122694 | GLIPR2 | 0.00 | 55.30 |
| ENSG00000137767 | SQOR | 0.00 | 54.10 |
| ENSG00000238113 | LINC01410 | 0.00 | 54.10 |
| ENSG00000075340 | ADD2 | 0.00 | 54.03 |
| ENSG00000106537 | TSPAN13 | 0.00 | 53.29 |
| ENSG00000167434 | CA4 | 0.00 | 53.20 |
| ENSG00000110931 | CAMKK2 | 0.00 | 52.60 |
| ENSG00000112053 | SLC26A8 | 0.00 | 52.50 |
| ENSG00000167207 | NOD2 | 0.00 | 52.29 |
| ENSG00000119655 | NPC2 | 0.00 | 52.16 |
| ENSG00000159346 | ADIPOR1 | 0.00 | 52.03 |
| ENSG00000109272 | PF4V1 | 0.00 | 51.63 |
| ENSG00000161911 | TREML1 | 0.00 | 51.60 |
| ENSG00000224032 | EPB41L4A-AS1 | 0.00 | 51.60 |
| ENSG00000135318 | NT5E | 0.00 | 51.51 |
| ENSG00000113319 | RASGRF2 | 0.00 | 51.38 |
| ENSG00000109743 | BST1 | 0.00 | 51.27 |
| ENSG00000172936 | MYD88 | 0.00 | 51.20 |
| ENSG00000196954 | CASP4 | 0.00 | 51.17 |
| ENSG00000185052 | SLC24A3 | 0.00 | 51.14 |
| ENSG00000288612 | ENSG00000288612 | 0.00 | 51.09 |
| ENSG00000163754 | GYG1 | 0.00 | 50.77 |
| ENSG00000065150 | IPO5 | 0.00 | 50.62 |
| ENSG00000161955 | TNFSF13 | 0.00 | 50.36 |
| ENSG00000271303 | SRXN1 | 0.00 | 49.60 |
| ENSG00000198176 | TFDP1 | 0.00 | 49.60 |
| ENSG00000138678 | GPAT3 | 0.00 | 49.43 |
| ENSG00000132386 | SERPINF1 | 0.00 | 49.26 |
| ENSG00000159128 | IFNGR2 | 0.00 | 49.10 |
| ENSG00000171604 | CXXC5 | 0.00 | 49.03 |
| ENSG00000113140 | SPARC | 0.00 | 49.00 |
| ENSG00000111962 | UST | 0.00 | 49.00 |
| ENSG00000136827 | TOR1A | 0.00 | 48.98 |
| ENSG00000173083 | HPSE | 0.00 | 48.70 |
| ENSG00000187554 | TLR5 | 0.00 | 48.60 |
| ENSG00000109854 | HTATIP2 | 0.00 | 48.50 |
| ENSG00000180316 | PNPLA1 | 0.00 | 48.20 |
| ENSG00000183621 | ZNF438 | 0.00 | 48.20 |
| ENSG00000156381 | ANKRD9 | 0.00 | 48.00 |
| ENSG00000123130 | ACOT9 | 0.00 | 47.97 |
| ENSG00000137752 | CASP1 | 0.00 | 47.80 |
| ENSG00000059804 | SLC2A3 | 0.00 | 47.70 |
| ENSG00000005961 | ITGA2B | 0.00 | 47.63 |
| ENSG00000128203 | ASPHD2 | 0.00 | 47.40 |
| ENSG00000147457 | CHMP7 | 0.00 | 47.30 |
| ENSG00000198182 | ZNF607 | 0.00 | 47.25 |
| ENSG00000105136 | ZNF419 | 0.00 | 47.07 |
| ENSG00000188242 | PP7080 | 0.00 | 47.06 |
| ENSG00000159445 | THEM4 | 0.00 | 47.00 |
| ENSG00000137198 | GMPR | 0.00 | 46.60 |
| ENSG00000113916 | BCL6 | 0.00 | 46.47 |
| ENSG00000128512 | DOCK4 | 0.00 | 46.33 |
| ENSG00000157551 | KCNJ15 | 0.00 | 46.13 |
| ENSG00000112062 | MAPK14 | 0.00 | 46.10 |
| ENSG00000117602 | RCAN3 | 0.00 | 46.10 |
| ENSG00000068971 | PPP2R5B | 0.00 | 46.10 |
| ENSG00000290104 | ENSG00000290104 | 0.00 | 46.10 |
| ENSG00000101335 | MYL9 | 0.00 | 45.87 |
| ENSG00000162367 | TAL1 | 0.00 | 45.80 |
| ENSG00000215788 | TNFRSF25 | 0.00 | 45.63 |
| ENSG00000176871 | WSB2 | 0.00 | 45.57 |
| ENSG00000120885 | CLU | 0.00 | 45.43 |
| ENSG00000288836 | ENSG00000288836 | 0.00 | 45.40 |
| ENSG00000150337 | FCGR1A | 0.00 | 44.87 |
| ENSG00000182541 | LIMK2 | 0.00 | 44.63 |
| ENSG00000132965 | ALOX5AP | 0.00 | 44.53 |
| ENSG00000143753 | DEGS1 | 0.00 | 44.47 |
| ENSG00000157800 | SLC37A3 | 0.00 | 44.40 |
| ENSG00000169228 | RAB24 | 0.00 | 44.30 |
| ENSG00000164849 | GPR146 | 0.00 | 44.30 |
| ENSG00000137509 | PRCP | 0.00 | 44.23 |
| ENSG00000121858 | TNFSF10 | 0.00 | 43.83 |
| ENSG00000128383 | APOBEC3A | 0.00 | 43.80 |
| ENSG00000233030 | ENSG00000233030 | 0.00 | 43.75 |
| ENSG00000186431 | FCAR | 0.00 | 43.57 |
| ENSG00000143119 | CD53 | 0.00 | 43.53 |
| ENSG00000224389 | C4B | 0.00 | 43.53 |
| ENSG00000070540 | WIPI1 | 0.00 | 43.40 |
| ENSG00000145685 | LHFPL2 | 0.00 | 43.30 |
| ENSG00000096063 | SRPK1 | 0.00 | 43.30 |
| ENSG00000244734 | HBB | 0.00 | 43.30 |
| ENSG00000108984 | MAP2K6 | 0.00 | 43.27 |
| ENSG00000073792 | IGF2BP2 | 0.00 | 43.27 |
| ENSG00000116793 | PHTF1 | 0.00 | 43.20 |
| ENSG00000186918 | ZNF395 | 0.00 | 43.17 |
| ENSG00000125952 | MAX | 0.00 | 43.00 |
| ENSG00000100100 | PIK3IP1 | 0.00 | 43.00 |
| ENSG00000063046 | EIF4B | 0.00 | 42.73 |
| ENSG00000246016 | LINC01513 | 0.00 | 42.70 |
| ENSG00000204420 | MPIG6B | 0.00 | 42.70 |
| ENSG00000163191 | S100A11 | 0.00 | 42.60 |
| ENSG00000165795 | NDRG2 | 0.00 | 42.57 |
| ENSG00000101474 | APMAP | 0.00 | 42.43 |
| ENSG00000216490 | IFI30 | 0.00 | 42.20 |
| ENSG00000160785 | SLC25A44 | 0.00 | 42.10 |
| ENSG00000099204 | ABLIM1 | 0.00 | 41.93 |
| ENSG00000130830 | MPP1 | 0.00 | 41.93 |
| ENSG00000186854 | TRABD2A | 0.00 | 41.77 |
| ENSG00000268500 | SIGLEC5 | 0.00 | 41.73 |
| ENSG00000167483 | NIBAN3 | 0.00 | 41.63 |
| ENSG00000111666 | CHPT1 | 0.00 | 41.43 |
| ENSG00000244731 | C4A | 0.00 | 41.43 |
| ENSG00000140743 | CDR2 | 0.00 | 41.33 |
| ENSG00000167208 | SNX20 | 0.00 | 41.30 |
| ENSG00000138798 | EGF | 0.00 | 41.20 |
| ENSG00000256235 | SMIM3 | 0.00 | 41.20 |
| ENSG00000081059 | TCF7 | 0.00 | 41.20 |
| ENSG00000287839 | ENSG00000287839 | 0.00 | 41.17 |
| ENSG00000121413 | ZSCAN18 | 0.00 | 41.10 |
| ENSG00000161381 | PLXDC1 | 0.00 | 41.10 |
| ENSG00000178809 | TRIM73 | 0.00 | 41.08 |
| ENSG00000231607 | DLEU2 | 0.00 | 41.07 |
| ENSG00000118508 | RAB32 | 0.00 | 40.97 |
| ENSG00000081177 | EXD2 | 0.00 | 40.96 |
| ENSG00000075413 | MARK3 | 0.00 | 40.93 |
| ENSG00000136156 | ITM2B | 0.00 | 40.90 |
| ENSG00000176225 | RTTN | 0.00 | 40.84 |
| ENSG00000111644 | ACRBP | 0.00 | 40.83 |
| ENSG00000145491 | ROPN1L | 0.00 | 40.80 |
| ENSG00000270210 | ENSG00000270210 | 0.00 | 40.75 |
| ENSG00000269404 | SPIB | 0.00 | 40.63 |
| ENSG00000165030 | NFIL3 | 0.00 | 40.60 |
| ENSG00000164236 | ANKRD33B | 0.00 | 40.55 |
| ENSG00000121060 | TRIM25 | 0.00 | 40.40 |
| ENSG00000102010 | BMX | 0.00 | 40.40 |
| ENSG00000151623 | NR3C2 | 0.00 | 40.23 |
| ENSG00000162894 | FCMR | 0.00 | 40.20 |
| ENSG00000187116 | LILRA5 | 0.00 | 40.10 |
| ENSG00000124374 | PAIP2B | 0.00 | 39.95 |
| ENSG00000197208 | SLC22A4 | 0.00 | 39.90 |
| ENSG00000228956 | SATB1-AS1 | 0.00 | 39.88 |
| ENSG00000132357 | CARD6 | 0.00 | 39.87 |
| ENSG00000163568 | AIM2 | 0.00 | 39.83 |
| ENSG00000225936 | SLC18A2-AS1 | 0.00 | 39.83 |
| ENSG00000158825 | CDA | 0.00 | 39.80 |
| ENSG00000181754 | AMIGO1 | 0.00 | 39.80 |
| ENSG00000158373 | H2BC5 | 0.00 | 39.77 |
| ENSG00000278828 | H3C10 | 0.00 | 39.57 |
| ENSG00000101842 | VSIG1 | 0.00 | 39.50 |
| ENSG00000148926 | ADM | 0.00 | 39.40 |
| ENSG00000068323 | TFE3 | 0.00 | 39.17 |
| ENSG00000144218 | AFF3 | 0.00 | 39.15 |
| ENSG00000158470 | B4GALT5 | 0.00 | 39.07 |
| ENSG00000288882 | ENSG00000288882 | 0.00 | 39.03 |
| ENSG00000135334 | AKIRIN2 | 0.00 | 38.90 |
| ENSG00000128218 | VPREB3 | 0.00 | 38.9 |
| ENSG00000121933 | TMIGD3 | 0.00 | 38.87 |
| ENSG00000106624 | AEBP10 | 0.00 | 38.85 |
| ENSG00000230615 | ENSG00000230615 | 0.00 | 38.73 |
| ENSG00000187037 | GPR141 | 0.00 | 38.50 |
| ENSG00000172197 | MBOAT1 | 0.00 | 38.47 |
| ENSG00000139266 | MARCHF9 | 0.00 | 38.27 |
| ENSG00000204397 | CARD16 | 0.00 | 38.07 |
| ENSG00000170909 | OSCAR | 0.00 | 38.03 |
| ENSG00000105835 | NAMPT | 0.00 | 37.97 |
| ENSG00000064601 | CTSA | 0.00 | 37.90 |
| ENSG00000162496 | DHRS3 | 0.00 | 37.82 |
| ENSG00000177663 | IL17RA | 0.00 | 37.80 |
| ENSG00000140391 | TSPAN3 | 0.00 | 37.73 |
| ENSG00000069974 | RAB27A | 0.00 | 37.67 |
| ENSG00000186205 | MTARC1 | 0.00 | 37.60 |
| ENSG00000168056 | LTBP3 | 0.00 | 37.50 |
| ENSG00000124225 | PMEPA1 | 0.00 | 37.47 |
| ENSG00000088298 | EDEM2 | 0.00 | 37.47 |
| ENSG00000119950 | MXI1 | 0.00 | 37.40 |
| ENSG00000125485 | DDX31 | 0.00 | 37.34 |
| ENSG00000149557 | FEZ1 | 0.00 | 37.22 |
| ENSG00000169896 | ITGAM | 0.00 | 37.17 |
| ENSG00000204386 | NEU1 | 0.00 | 37.07 |
| ENSG00000133317 | LGALS12 | 0.00 | 36.93 |
| ENSG00000013583 | HEBP1 | 0.00 | 36.93 |
| ENSG00000184271 | POU6F1 | 0.00 | 36.80 |
| ENSG00000085465 | OVGP1 | 0.00 | 36.68 |
| ENSG00000005471 | ABCB4 | 0.00 | 36.65 |
| ENSG00000196793 | ZNF239 | 0.00 | 36.60 |
| ENSG00000160201 | U2AF1 | 0.00 | 36.53 |
| ENSG00000112182 | BACH2 | 0.00 | 36.50 |
| ENSG00000187650 | VMAC | 0.00 | 36.41 |
| ENSG00000112299 | VNN1 | 0.00 | 36.30 |
| ENSG00000138185 | ENTPD1 | 0.00 | 36.30 |
| ENSG00000135636 | DYSF | 0.00 | 36.20 |
| ENSG00000163162 | RNF149 | 0.00 | 36.13 |
| ENSG00000101160 | CTSZ | 0.00 | 36.10 |
| ENSG00000108861 | DUSP3 | 0.00 | 36.00 |
| ENSG00000116701 | NCF2 | 0.00 | 35.90 |
| ENSG00000185875 | THNSL1 | 0.00 | 35.83 |
| ENSG00000124762 | CDKN1A | 0.00 | 35.83 |
| ENSG00000158869 | FCER1G | 0.00 | 35.73 |
| ENSG00000139998 | RAB15 | 0.00 | 35.73 |
| ENSG00000120217 | CD274 | 0.00 | 35.68 |
| ENSG00000132274 | TRIM22 | 0.00 | 35.67 |
| ENSG00000165943 | MOAP1 | 0.00 | 35.67 |
| ENSG00000095303 | PTGS1 | 0.00 | 35.57 |
| ENSG00000124098 | FAM210B | 0.00 | 35.53 |
| ENSG00000127804 | METTL16 | 0.00 | 35.40 |
| ENSG00000249992 | TMEM158 | 0.00 | 35.37 |
| ENSG00000272914 | ENSG00000272914 | 0.00 | 35.30 |
| ENSG00000125744 | RTN2 | 0.00 | 35.30 |
| ENSG00000198106 | ENSG00000198106 | 0.00 | 35.25 |
| ENSG00000149257 | SERPINH1 | 0.00 | 35.25 |
| ENSG00000115271 | GCA | 0.00 | 35.20 |
| ENSG00000175471 | MCTP1 | 0.00 | 35.13 |
| ENSG00000102230 | PCYT1B | 0.00 | 35.10 |
| ENSG00000135404 | CD63 | 0.00 | 35.00 |
| ENSG00000166341 | DCHS1 | 0.00 | 35.00 |
| ENSG00000211459 | MT-RNR1 | 0.00 | 34.97 |
| ENSG00000114784 | EIF1B | 0.00 | 34.97 |
| ENSG00000140853 | NLRC5 | 0.00 | 34.93 |
| ENSG00000250608 | NUDT16-DT | 0.00 | 34.93 |
| ENSG00000163421 | PROK2 | 0.00 | 34.87 |
| ENSG00000164713 | BRI3 | 0.00 | 34.70 |
| ENSG00000136026 | CKAP4 | 0.00 | 34.70 |
| ENSG00000151726 | ACSL1 | 0.00 | 34.67 |
| ENSG00000225873 | C3orf86 | 0.00 | 34.57 |
| ENSG00000236144 | TMEM147-AS1 | 0.00 | 34.55 |
| ENSG00000214212 | C19orf38 | 0.00 | 34.53 |
| ENSG00000176293 | ZNF135 | 0.00 | 34.47 |
| ENSG00000134548 | SPX | 0.00 | 34.45 |
| ENSG00000162630 | B3GALT2 | 0.00 | 34.43 |
| ENSG00000198932 | GPRASP1 | 0.00 | 34.42 |
| ENSG00000152804 | HHEX | 0.00 | 34.33 |

TABLE 3

| ensemble ID | gene symbol | AA score |
|---|---|---|
| ENSG00000128656 | CHN1 | 52.02 |
| ENSG00000273314 | ENSG00000273314 | 43.66 |
| ENSG00000091181 | IL5RA | 41.47 |
| ENSG00000048162 | NOP16 | 24.82 |
| ENSG00000135414 | GDF11 | 24.60 |
| ENSG00000204444 | APOM | 23.40 |
| ENSG00000144290 | SLC4A10 | 23.03 |
| ENSG00000164663 | USP49 | 20.00 |
| ENSG00000123358 | NR4A1 | 19.86 |
| ENSG00000020181 | ADGRA2 | 18.88 |
| ENSG00000158050 | DUSP2 | 18.67 |
| ENSG00000203705 | TATDN3 | 16.13 |
| ENSG00000166035 | LIPC | 15.62 |
| ENSG00000120738 | EGR1 | 15.40 |
| ENSG00000186081 | KRT5 | 12.93 |
| ENSG00000174099 | MSRB3 | 12.47 |
| ENSG00000141560 | FN3KRP | 12.19 |
| ENSG00000140287 | HDC | 11.70 |
| ENSG00000240403 | KIR3DL2 | 10.85 |
| ENSG00000091640 | SPAG7 | 10.47 |
| ENSG00000163661 | PTX3 | 10.43 |
| ENSG00000144214 | LYG1 | 10.25 |
| ENSG00000170854 | RIOX2 | 10.19 |
| ENSG00000257027 | ENSG00000257027 | 9.80 |
| ENSG00000138758 | SEPTIN11 | 9.76 |
| ENSG00000177374 | HIC1 | 8.43 |
| ENSG00000198960 | ARMCX6 | 7.10 |
| ENSG00000073849 | ST6GAL1 | 7.03 |
| ENSG00000135622 | SEMA4F | 6.97 |
| ENSG00000187134 | AKR1C1 | 6.88 |
| ENSG00000226979 | LTA | 6.80 |
| ENSG00000265118 | ENSG00000265118 | 6.13 |
| ENSG00000288082 | ENSG00000288082 | 5.89 |
| ENSG00000198018 | ENTPD7 | 5.87 |
| ENSG00000165646 | SLC18A2 | 5.53 |
| ENSG00000244607 | CCDC13 | 5.35 |
| ENSG00000124201 | ZNFX1 | 5.30 |
| ENSG00000154856 | APCDD1 | 5.27 |
| ENSG00000112486 | CCR6 | 5.15 |
| ENSG00000151366 | NDUFC2 | 5.00 |
| ENSG00000119729 | RHOQ | 4.90 |
| ENSG00000141664 | ZCCHC2 | 4.40 |
| ENSG00000130821 | SLC6A8 | 4.27 |
| ENSG00000204267 | TAP2 | 4.20 |
| ENSG00000183864 | TOB2 | 4.00 |
| ENSG00000139624 | CERS5 | 3.80 |
| ENSG00000055332 | EIF2AK2 | 3.77 |
| ENSG00000100813 | ACIN1 | 3.59 |
| ENSG00000100441 | KHNYN | 3.57 |
| ENSG00000143367 | TUFT1 | 3.50 |
| ENSG00000124942 | AHNAK | 3.43 |
| ENSG00000139055 | ERP27 | 3.43 |
| ENSG00000148346 | LCN2 | 3.40 |
| ENSG00000198663 | C6orf89 | 3.40 |
| ENSG00000099282 | TSPAN15 | 3.37 |
| ENSG00000198019 | FCGR1BP | 3.20 |
| ENSG00000225938 | S1PR1-DT | 3.10 |
| ENSG00000225101 | OR52K3P | 2.47 |
| ENSG00000068400 | GRIPAP1 | 2.40 |
| ENSG00000115998 | C2orf42 | 2.27 |
| ENSG00000136826 | KLF4 | 2.10 |
| ENSG00000129084 | PSMA1 | 2.10 |
| ENSG00000110060 | PUS3 | 2.00 |
| ENSG00000197555 | SIPA1L1 | 1.90 |
| ENSG00000141252 | VPS53 | 1.90 |
| ENSG00000132185 | FCRLA | 1.80 |
| ENSG00000145919 | BOD1 | 1.80 |
| ENSG00000184983 | NDUFA6 | 1.80 |
| ENSG00000226777 | FAM30A | 1.77 |
| ENSG00000084110 | HAL | 1.77 |
| ENSG00000182196 | ARL6IP4 | 1.47 |
| ENSG00000213903 | LTB4R | 1.40 |
| ENSG00000141337 | ARSG | 1.33 |
| ENSG00000178537 | SLC25A20 | 1.23 |
| ENSG00000239998 | LILRA2 | 1.20 |
| ENSG00000198937 | CCDC167 | 1.07 |
| ENSG00000166947 | EPB42 | 0.00 |
| ENSG00000137198 | GMPR | 0.00 |
| ENSG00000211459 | MT-RNR1 | 0.00 |
| ENSG00000284882 | ENSG00000284882 | 0.00 |
| ENSG00000117262 | GPR89A | 0.00 |
| ENSG00000206177 | HBM | 0.00 |
| ENSG00000099901 | RANBP1 | 0.00 |
| ENSG00000260923 | LINC02193 | 0.00 |

TABLE 4

| ensemble ID | gene symbol | CRC score |
|---|---|---|
| ENSG00000115828 | QPCT | 57.33 |
| ENSG00000204580 | DDR1 | 55.65 |
| ENSG00000172936 | MYD88 | 51.20 |
| ENSG00000163754 | GYG1 | 50.77 |
| ENSG00000161955 | TNFSF13 | 50.36 |
| ENSG00000198182 | ZNF607 | 47.25 |
| ENSG00000105426 | PTPRS | 43.69 |
| ENSG00000096063 | SRPK1 | 43.30 |
| ENSG00000186918 | ZNF395 | 43.17 |
| ENSG00000100100 | PIK3IP1 | 43.00 |
| ENSG00000101474 | APMAP | 42.43 |
| ENSG00000111666 | CHPT1 | 41.43 |
| ENSG00000176225 | RTTN | 40.84 |
| ENSG00000187116 | LILRA5 | 40.10 |
| ENSG00000197208 | SLC22A4 | 39.90 |
| ENSG00000158825 | CDA | 39.80 |
| ENSG00000230615 | ENSG00000230615 | 38.73 |
| ENSG00000172197 | MBOAT1 | 38.47 |
| ENSG00000119950 | MXI1 | 37.40 |
| ENSG00000113273 | ARSB | 36.74 |
| ENSG00000160201 | U2AF1 | 36.53 |
| ENSG00000124098 | FAM210B | 35.53 |
| ENSG00000163565 | IFI16 | 35.33 |
| ENSG00000060138 | YBX3 | 34.27 |
| ENSG00000282988 | ENSG00000282988 | 33.87 |
| ENSG00000157168 | NRG1 | 33.80 |
| ENSG00000152766 | ANKRD22 | 33.22 |
| ENSG00000103005 | USB1 | 32.47 |
| ENSG00000023318 | ERP44 | 32.40 |
| ENSG00000000938 | FGR | 32.33 |
| ENSG00000130821 | SLC6A8 | 31.90 |
| ENSG00000105486 | LIG1 | 31.80 |
| ENSG00000129351 | ILF3 | 31.77 |
| ENSG00000133056 | PIK3C2B | 31.35 |
| ENSG00000274536 | MIR223HG | 30.73 |
| ENSG00000142089 | IFITM3 | 30.20 |
| ENSG00000153107 | ANAPC1 | 29.93 |
| ENSG00000118518 | RNF146 | 29.47 |
| ENSG00000130066 | SAT1 | 29.07 |
| ENSG00000025708 | TYMP | 28.87 |
| ENSG00000139178 | C1RL | 28.40 |
| ENSG00000126368 | NR1D1 | 27.95 |
| ENSG00000108387 | SEPTIN4 | 27.68 |
| ENSG00000183785 | TUBA8 | 26.60 |
| ENSG00000186204 | CYP4F12 | 26.00 |
| ENSG00000135604 | STX11 | 25.60 |
| ENSG00000005249 | PRKAR2B | 25.17 |
| ENSG00000173559 | NABP1 | 25.13 |
| ENSG00000198876 | DCAF12 | 25.10 |
| ENSG00000116005 | PCYOX1 | 24.99 |
| ENSG00000146416 | AIG1 | 23.38 |
| ENSG00000160593 | JAML | 23.27 |
| ENSG00000145936 | KCNMB1 | 23.17 |
| ENSG00000171843 | MLLT3 | 22.00 |
| ENSG00000174669 | SLC29A2 | 21.97 |
| ENSG00000160299 | PCNT | 21.80 |
| ENSG00000160049 | DFFA | 21.80 |
| ENSG00000166900 | STX3 | 21.07 |
| ENSG00000108309 | RUNDC3A | 21.00 |
| ENSG00000133318 | RTN3 | 20.80 |
| ENSG00000132334 | PTPRE | 20.30 |
| ENSG00000139436 | GIT2 | 19.60 |
| ENSG00000180596 | H2BC4 | 19.53 |
| ENSG00000198858 | R3HDM4 | 18.83 |
| ENSG00000152465 | NMT2 | 18.70 |
| ENSG00000258819 | LINC02289 | 18.40 |
| ENSG00000166947 | EPB42 | 18.20 |
| ENSG00000268027 | ENSG00000268027 | 17.93 |
| ENSG00000160991 | ORAI2 | 16.70 |

TABLE 5

| | | | | | selection |
|---|---|---|---|---|---|
| ensemble ID | gene symbol | comparison | deseq_logFC | deseq_adj_p | method |
| ENSG00000184178 | SCFD2 | earlyCRC_CON | −0.21 | 0.05 | univariant |
| ENSG00000163933 | RFT1 | earlyCRC_CON | 0.15 | 0.07 | multivariant |
| ENSG00000054277 | OPN3 | earlyCRC_CON | −0.09 | 0.23 | multivariant |
| ENSG00000163597 | SNHG16 | earlyCRC_CON | −0.08 | 0.27 | multivariant |
| ENSG00000186687 | LYRM7 | earlyCRC_CON | −0.08 | 0.29 | multivariant |
| ENSG00000104921 | FCER2 | earlyCRC_CON | −0.07 | 0.30 | multivariant |
| ENSG00000185973 | TMLHE | earlyCRC_CON | −0.07 | 0.34 | multivariant |
| ENSG00000121104 | FAM117A | earlyCRC_CON | 0.06 | 0.45 | multivariant |
| ENSG00000173531 | MST1 | earlyCRC_CON | −0.35 | 0.02 | univariant |
| ENSG00000107890 | ANKRD26 | earlyCRC_CON | −0.16 | 0.04 | univariant |
| ENSG00000166821 | PEX11A | earlyCRC_CON | −0.17 | 0.07 | multivariant |
| ENSG00000138303 | ASCC1 | earlyCRC_CON | −0.11 | 0.15 | multivariant |
| ENSG00000140987 | ZSCAN32 | earlyCRC_CON | −0.09 | 0.23 | multivariant |
| ENSG00000157014 | TATDN2 | earlyCRC_CON | −0.06 | 0.40 | multivariant |
| ENSG00000131116 | ZNF428 | earlyCRC_CON | −0.05 | 0.55 | multivariant |
| ENSG00000136770 | DNAJC1 | earlyCRC_CON | 0.04 | 0.64 | multivariant |
| ENSG00000136630 | HLX | earlyCRC_CON | 0.03 | 0.76 | multivariant |
| ENSG00000204536 | CCHCR1 | earlyCRC_CON | −0.03 | 0.77 | multivariant |
| ENSG00000163803 | PLB1 | groupAA_CON | −0.09 | 0.16 | multivariant |
| ENSG00000112137 | PHACTR1 | groupAA_CON | 0.19 | 0.07 | multivariant |
| ENSG00000102893 | PHKB | groupAA_CON | 0.05 | 0.30 | multivariant |
| ENSG00000102898 | NUTF2 | groupAA_CON | −0.06 | 0.32 | multivariant |
| ENSG00000129255 | MPDU1 | groupAA_CON | −0.05 | 0.46 | multivariant |
| ENSG00000065485 | PDIA5 | groupAA_CON | −0.06 | 0.22 | multivariant |
| ENSG00000237649 | KIFC1 | groupAA_CON | −0.05 | 0.30 | multivariant |
| ENSG00000004809 | SLC22A16 | groupAA_CON | −0.05 | 0.31 | multivariant |
| ENSG00000072422 | RHOBTB1 | groupAA_CON | 0.05 | 0.31 | multivariant |
| ENSG00000134531 | EMP1 | groupAA_CON | −1.07 | 0.00 | univariant |
| ENSG00000114026 | OGG1 | groupAA_CON | −0.06 | 0.25 | multivariant |
| ENSG00000130921 | C12orf65 | groupAA_CON | 0.06 | 0.31 | multivariant |
| ENSG00000141076 | UTP4 | groupAA_CON | −0.06 | 0.32 | multivariant |
| ENSG00000172053 | QARS | groupAA_CON | −0.06 | 0.34 | multivariant |
| ENSG00000135930 | EIF4E2 | groupAA_CON | −0.05 | 0.34 | multivariant |
| ENSG00000157020 | SEC13 | groupAA_CON | −0.05 | 0.43 | multivariant |
| ENSG00000112799 | LY86 | groupAA_CON | −0.04 | 0.47 | multivariant |
| ENSG00000162869 | PPP1R21 | groupAA_CON | 0.04 | 0.51 | multivariant |
| ENSG00000151491 | EPS8 | groupAA_CON | −0.75 | 0.01 | univariant |
| ENSG00000099139 | PCSK5 | groupAA_CON | 0.26 | 0.05 | univariant |
| ENSG00000020577 | SAMD4A | groupAA_CON | −0.38 | 0.06 | univariant |
| ENSG00000184924 | PTRHD1 | groupAA_CON | −0.14 | 0.07 | multivariant |
| ENSG00000071242 | RPS6KA2 | groupAA_CON | −0.13 | 0.10 | multivariant |
| ENSG00000163629 | PTPN13 | groupAA_CON | −0.18 | 0.12 | multivariant |
| ENSG00000197782 | ZNF780A | groupAA_CON | −0.08 | 0.16 | multivariant |
| ENSG00000139291 | TMEM19 | groupAA_CON | −0.08 | 0.17 | multivariant |
| ENSG00000167005 | NUDT21 | groupAA_CON | −0.08 | 0.18 | multivariant |
| ENSG00000239382 | ALKBH6 | groupAA_CON | −0.06 | 0.24 | multivariant |
| ENSG00000269893 | SNHG8 | groupAA_CON | −0.06 | 0.24 | multivariant |
| ENSG00000132623 | ANKEF1 | groupAA_CON | 0.06 | 0.24 | multivariant |
| ENSG00000205593 | DENND6B | groupAA_CON | −0.06 | 0.25 | multivariant |
| ENSG00000117691 | NENF | groupAA_CON | −0.06 | 0.25 | multivariant |
| ENSG00000154518 | ATP5MC3 | groupAA_CON | −0.06 | 0.25 | multivariant |
| ENSG00000175575 | PAAF1 | groupAA_CON | −0.06 | 0.27 | multivariant |
| ENSG00000140691 | ARMC5 | groupAA_CON | −0.05 | 0.28 | multivariant |
| ENSG00000100577 | GSTZ1 | groupAA_CON | −0.05 | 0.30 | multivariant |
| ENSG00000100181 | TPTEP1 | groupAA_CON | 0.05 | 0.31 | multivariant |
| ENSG00000111640 | GAPDH | groupAA_CON | −0.05 | 0.33 | multivariant |
| ENSG00000119686 | FLVCR2 | groupAA_CON | −0.05 | 0.33 | multivariant |
| ENSG00000178741 | COX5A | groupAA_CON | −0.05 | 0.33 | multivariant |
| ENSG00000148358 | GPR107 | groupAA_CON | 0.06 | 0.34 | multivariant |
| ENSG00000197037 | ZSCAN25 | groupAA_CON | 0.05 | 0.34 | multivariant |
| ENSG00000214530 | STARD10 | groupAA_CON | −0.05 | 0.35 | multivariant |
| ENSG00000113643 | RARS | groupAA_CON | −0.06 | 0.35 | multivariant |
| ENSG00000163818 | LZTFL1 | groupAA_CON | −0.05 | 0.36 | multivariant |
| ENSG00000132846 | ZBED3 | groupAA_CON | −0.04 | 0.36 | multivariant |
| ENSG00000197363 | ZNF517 | groupAA_CON | −0.05 | 0.37 | multivariant |
| ENSG00000255198 | SNHG9 | groupAA_CON | −0.04 | 0.38 | multivariant |
| ENSG00000185721 | DRG1 | groupAA_CON | −0.05 | 0.38 | multivariant |
| ENSG00000258315 | C17orf49 | groupAA_CON | −0.05 | 0.40 | multivariant |
| ENSG00000111669 | TPI1 | groupAA_CON | −0.05 | 0.40 | multivariant |
| ENSG00000126432 | PRDX5 | groupAA_CON | −0.04 | 0.41 | multivariant |
| ENSG00000049618 | ARID1B | groupAA_CON | 0.05 | 0.45 | multivariant |
| ENSG00000171634 | BPTF | groupAA_CON | 0.04 | 0.48 | multivariant |
| ENSG00000168672 | FAM84B | groupAA_CON | 0.02 | 0.57 | multivariant |
| ENSG00000267520 | AC010733.2 | groupAA_CON | 0.02 | 0.58 | multivariant |
| ENSG00000283674 | AC068587.4 | groupAA_CON | 0.02 | 0.68 | multivariant |
| ENSG00000075239 | ACAT1 | groupAA_CON | −0.02 | 0.71 | multivariant |
| ENSG00000158796 | DEDD | groupAA_CON | 0.02 | 0.77 | multivariant |
| ENSG00000139684 | ESD | groupAA_CON | −0.01 | 0.83 | multivariant |
| ENSG00000104129 | DJC17 | groupAA_CON | −0.06 | 0.19 | multivariant |
| ENSG00000128731 | HERC2 | groupAA_CON | 0.03 | 0.54 | multivariant |
| ENSG00000114491 | UMPS | groupAA_CON | −0.05 | 0.30 | multivariant |
| ENSG00000177106 | EPS8L2 | groupAA_CON | −0.49 | 0.01 | univariant |
| ENSG00000214756 | CSKMT | groupAA_CON | −0.36 | 0.05 | univariant |
| ENSG00000162572 | SCNN1D | groupAA_CON | −0.18 | 0.10 | multivariant |
| ENSG00000123104 | ITPR2 | groupAA_CON | −0.18 | 0.10 | multivariant |
| ENSG00000228594 | FNDC10 | groupAA_CON | −0.08 | 0.14 | multivariant |
| ENSG00000125843 | AP5S1 | groupAA_CON | −0.08 | 0.14 | multivariant |
| ENSG00000204271 | SPIN3 | groupAA_CON | 0.07 | 0.15 | multivariant |
| ENSG00000170348 | TMED10 | groupAA_CON | −0.07 | 0.15 | multivariant |
| ENSG00000176894 | PXMP2 | groupAA_CON | −0.07 | 0.16 | multivariant |
| ENSG00000100197 | CYP2D6 | groupAA_CON | −0.08 | 0.16 | multivariant |
| ENSG00000159792 | PSKH1 | groupAA_CON | −0.07 | 0.19 | multivariant |
| ENSG00000099341 | PSMD8 | groupAA_CON | −0.07 | 0.19 | multivariant |
| ENSG00000110442 | COMMD9 | groupAA_CON | −0.06 | 0.20 | multivariant |
| ENSG00000161960 | EIF4A1 | groupAA_CON | −0.07 | 0.21 | multivariant |
| ENSG00000140365 | COMMD4 | groupAA_CON | −0.06 | 0.22 | multivariant |
| ENSG00000185527 | PDE6G | groupAA_CON | −0.06 | 0.24 | multivariant |
| ENSG00000167088 | SNRPD1 | groupAA_CON | −0.06 | 0.24 | multivariant |
| ENSG00000177854 | TMEM187 | groupAA_CON | −0.05 | 0.30 | multivariant |
| ENSG00000107960 | STN1 | groupAA_CON | −0.06 | 0.30 | multivariant |
| ENSG00000099800 | TIMM13 | groupAA_CON | −0.05 | 0.30 | multivariant |
| ENSG00000162222 | TTC9C | groupAA_CON | −0.05 | 0.32 | multivariant |
| ENSG00000151062 | CAC2D4 | groupAA_CON | −0.05 | 0.33 | multivariant |
| ENSG00000143322 | ABL2 | groupAA_CON | 0.06 | 0.33 | multivariant |
| ENSG00000221994 | ZNF630 | groupAA_CON | 0.05 | 0.33 | multivariant |
| ENSG00000068001 | HYAL2 | groupAA_CON | −0.05 | 0.33 | multivariant |
| ENSG00000114054 | PCCB | groupAA_CON | −0.06 | 0.34 | multivariant |
| ENSG00000125877 | ITPA | groupAA_CON | −0.05 | 0.34 | multivariant |
| ENSG00000119684 | MLH3 | groupAA_CON | −0.04 | 0.35 | multivariant |
| ENSG00000122550 | KLHL7 | groupAA_CON | −0.04 | 0.37 | multivariant |
| ENSG00000163596 | ICA1L | groupAA_CON | 0.04 | 0.37 | multivariant |
| ENSG00000115109 | EPB41L5 | groupAA_CON | 0.04 | 0.38 | multivariant |
| ENSG00000285793 | AC125232.2 | groupAA_CON | 0.05 | 0.40 | multivariant |
| ENSG00000103274 | NUBP1 | groupAA_CON | −0.05 | 0.41 | multivariant |
| ENSG00000119718 | EIF2B2 | groupAA_CON | −0.05 | 0.44 | multivariant |
| ENSG00000007129 | CEACAM21 | groupAA_CON | −0.04 | 0.47 | multivariant |
| ENSG00000079482 | OPHN1 | groupAA_CON | −0.02 | 0.69 | multivariant |
| ENSG00000187122 | SLIT1 | groupAA_CON | −1.12 | 0.00 | univariant |
| ENSG00000108375 | RNF43 | groupAA_CON | −0.82 | 0.00 | univariant |
| ENSG00000146350 | TBC1D32 | groupAA_CON | 0.67 | 0.01 | univariant |
| ENSG00000177301 | KC2 | groupAA_CON | 0.61 | 0.02 | univariant |
| ENSG00000103995 | CEP152 | groupAA_CON | 0.48 | 0.02 | univariant |
| ENSG00000072415 | MPP5 | groupAA_CON | 0.29 | 0.05 | univariant |
| ENSG00000118514 | ALDH8A1 | groupAA_CON | 0.39 | 0.05 | univariant |
| ENSG00000216937 | CCDC7 | groupAA_CON | 0.29 | 0.06 | multivariant |
| ENSG00000248905 | FMN1 | groupAA_CON | −0.28 | 0.07 | multivariant |
| ENSG00000125733 | TRIP10 | groupAA_CON | 0.15 | 0.10 | multivariant |
| ENSG00000189171 | S100A13 | groupAA_CON | −0.15 | 0.11 | multivariant |
| ENSG00000119943 | PYROXD2 | groupAA_CON | 0.10 | 0.12 | multivariant |
| ENSG00000142583 | SLC2A5 | groupAA_CON | −0.18 | 0.12 | multivariant |
| ENSG00000111452 | ADGRD1 | groupAA_CON | −0.15 | 0.14 | multivariant |
| ENSG00000267575 | AC006504.5 | groupAA_CON | −0.07 | 0.15 | multivariant |
| ENSG00000117411 | B4GALT2 | groupAA_CON | −0.10 | 0.15 | multivariant |
| ENSG00000117010 | ZNF684 | groupAA_CON | 0.09 | 0.17 | multivariant |
| ENSG00000106123 | EPHB6 | groupAA_CON | −0.08 | 0.17 | multivariant |
| ENSG00000169814 | BTD | groupAA_CON | −0.07 | 0.17 | multivariant |
| ENSG00000268975 | MIA-RAB4B | groupAA_CON | −0.09 | 0.17 | multivariant |
| ENSG00000157349 | DDX19B | groupAA_CON | −0.07 | 0.18 | multivariant |
| ENSG00000182903 | ZNF721 | groupAA_CON | −0.07 | 0.18 | multivariant |
| ENSG00000167861 | HID1 | groupAA_CON | −0.09 | 0.18 | multivariant |
| ENSG00000251364 | AC107884.1 | groupAA_CON | 0.08 | 0.18 | multivariant |
| ENSG00000134013 | LOXL2 | groupAA_CON | −0.09 | 0.19 | multivariant |
| ENSG00000137563 | GGH | groupAA_CON | −0.08 | 0.19 | multivariant |
| ENSG00000008086 | CDKL5 | groupAA_CON | 0.07 | 0.19 | multivariant |
| ENSG00000104660 | LEPROTL1 | groupAA_CON | −0.06 | 0.19 | multivariant |
| ENSG00000182185 | RAD51B | groupAA_CON | −0.07 | 0.21 | multivariant |
| ENSG00000010256 | UQCRC1 | groupAA_CON | −0.06 | 0.21 | multivariant |
| ENSG00000069869 | NEDD4 | groupAA_CON | 0.06 | 0.22 | multivariant |
| ENSG00000196712 | NF1 | groupAA_CON | −0.07 | 0.22 | multivariant |
| ENSG00000155966 | AFF2 | groupAA_CON | −0.07 | 0.22 | multivariant |
| ENSG00000120254 | MTHFD1L | groupAA_CON | 0.07 | 0.22 | multivariant |
| ENSG00000042317 | SPATA7 | groupAA_CON | −0.06 | 0.22 | multivariant |
| ENSG00000106069 | CHN2 | groupAA_CON | −0.06 | 0.23 | multivariant |
| ENSG00000065802 | ASB1 | groupAA_CON | −0.06 | 0.23 | multivariant |
| ENSG00000272084 | AL137127.1 | groupAA_CON | −0.06 | 0.25 | multivariant |
| ENSG00000166575 | TMEM135 | groupAA_CON | −0.06 | 0.25 | multivariant |
| ENSG00000142945 | KIF2C | groupAA_CON | −0.06 | 0.25 | multivariant |
| ENSG00000072958 | AP1M1 | groupAA_CON | −0.06 | 0.26 | multivariant |
| ENSG00000165959 | CLMN | groupAA_CON | −0.06 | 0.27 | multivariant |
| ENSG00000131437 | KIF3A | groupAA_CON | 0.05 | 0.27 | multivariant |
| ENSG00000160972 | PPP1R16A | groupAA_CON | −0.06 | 0.28 | multivariant |
| ENSG00000225339 | AL354740.1 | groupAA_CON | −0.06 | 0.28 | multivariant |
| ENSG00000088881 | EBF4 | groupAA_CON | −0.06 | 0.29 | multivariant |
| ENSG00000269514 | AC024257.3 | groupAA_CON | 0.05 | 0.29 | multivariant |
| ENSG00000196476 | C20orf96 | groupAA_CON | −0.05 | 0.29 | multivariant |
| ENSG00000205464 | ATP6AP1L | groupAA_CON | −0.06 | 0.30 | multivariant |
| ENSG00000101220 | C20orf27 | groupAA_CON | −0.05 | 0.30 | multivariant |
| ENSG00000171425 | ZNF581 | groupAA_CON | −0.05 | 0.30 | multivariant |
| ENSG00000263335 | AF001548.2 | groupAA_CON | −0.05 | 0.30 | multivariant |
| ENSG00000139289 | PHLDA1 | groupAA_CON | 0.05 | 0.30 | multivariant |
| ENSG00000116198 | CEP104 | groupAA_CON | −0.05 | 0.30 | multivariant |
| ENSG00000144283 | PKP4 | groupAA_CON | 0.05 | 0.30 | multivariant |
| ENSG00000181019 | NQO1 | groupAA_CON | −0.05 | 0.30 | multivariant |
| ENSG00000147804 | SLC39A4 | groupAA_CON | −0.05 | 0.30 | multivariant |
| ENSG00000171703 | TCEA2 | groupAA_CON | −0.05 | 0.30 | multivariant |
| ENSG00000010626 | LRRC23 | groupAA_CON | −0.05 | 0.30 | multivariant |
| ENSG00000229043 | AC091729.3 | groupAA_CON | −0.05 | 0.30 | multivariant |
| ENSG00000135678 | CPM | groupAA_CON | −0.05 | 0.30 | multivariant |
| ENSG00000135540 | NHSL1 | groupAA_CON | 0.05 | 0.30 | multivariant |
| ENSG00000145284 | SCD5 | groupAA_CON | 0.05 | 0.30 | multivariant |
| ENSG00000078967 | UBE2D4 | groupAA_CON | −0.05 | 0.30 | multivariant |
| ENSG00000109501 | WFS1 | groupAA_CON | −0.05 | 0.31 | multivariant |
| ENSG00000198707 | CEP290 | groupAA_CON | 0.05 | 0.31 | multivariant |
| ENSG00000117724 | CENPF | groupAA_CON | −0.05 | 0.31 | multivariant |
| ENSG00000064201 | TSPAN32 | groupAA_CON | −0.05 | 0.32 | multivariant |
| ENSG00000131165 | CHMP1A | groupAA_CON | −0.06 | 0.32 | multivariant |
| ENSG00000174442 | ZWILCH | groupAA_CON | −0.05 | 0.32 | multivariant |
| ENSG00000040487 | PQLC2 | groupAA_CON | −0.06 | 0.33 | multivariant |
| ENSG00000160117 | ANKLE1 | groupAA_CON | −0.05 | 0.33 | multivariant |
| ENSG00000090263 | MRPS33 | groupAA_CON | −0.05 | 0.33 | multivariant |
| ENSG00000197858 | GPAA1 | groupAA_CON | −0.05 | 0.33 | multivariant |
| ENSG00000136813 | ECPAS | groupAA_CON | −0.05 | 0.33 | multivariant |
| ENSG00000159761 | C16orf86 | groupAA_CON | −0.05 | 0.33 | multivariant |
| ENSG00000121152 | NCAPH | groupAA_CON | −0.05 | 0.33 | multivariant |
| ENSG00000167965 | MLST8 | groupAA_CON | −0.05 | 0.33 | multivariant |
| ENSG00000182621 | PLCB1 | groupAA_CON | 0.05 | 0.33 | multivariant |
| ENSG00000213145 | CRIP1 | groupAA_CON | −0.05 | 0.34 | multivariant |
| ENSG00000144589 | STK11IP | groupAA_CON | −0.05 | 0.34 | multivariant |
| ENSG00000223547 | ZNF844 | groupAA_CON | 0.05 | 0.34 | multivariant |
| ENSG00000126870 | WDR60 | groupAA_CON | 0.04 | 0.34 | multivariant |
| ENSG00000171159 | C9orf16 | groupAA_CON | −0.05 | 0.34 | multivariant |
| ENSG00000156232 | WHAMM | groupAA_CON | 0.06 | 0.34 | multivariant |
| ENSG00000106266 | SNX8 | groupAA_CON | −0.04 | 0.34 | multivariant |
| ENSG00000133027 | PEMT | groupAA_CON | −0.05 | 0.34 | multivariant |
| ENSG00000177685 | CRACR2B | groupAA_CON | −0.04 | 0.34 | multivariant |
| ENSG00000172638 | EFEMP2 | groupAA_CON | −0.05 | 0.34 | multivariant |
| ENSG00000165923 | AGBL2 | groupAA_CON | 0.04 | 0.34 | multivariant |
| ENSG00000259529 | AL136295.5 | groupAA_CON | 0.05 | 0.34 | multivariant |
| ENSG00000139546 | TARBP2 | groupAA_CON | −0.06 | 0.34 | multivariant |
| ENSG00000152082 | MZT2B | groupAA_CON | −0.04 | 0.34 | multivariant |
| ENSG00000172893 | DHCR7 | groupAA_CON | −0.04 | 0.34 | multivariant |
| ENSG00000068097 | HEATR6 | groupAA_CON | 0.05 | 0.34 | multivariant |
| ENSG00000153006 | SREK1IP1 | groupAA_CON | −0.04 | 0.34 | multivariant |
| ENSG00000177570 | SAMD12 | groupAA_CON | 0.05 | 0.35 | multivariant |
| ENSG00000148481 | MINDY3 | groupAA_CON | 0.04 | 0.35 | multivariant |
| ENSG00000231752 | EMBP1 | groupAA_CON | −0.04 | 0.35 | multivariant |
| ENSG00000141349 | G6PC3 | groupAA_CON | −0.05 | 0.35 | multivariant |
| ENSG00000187778 | MCRS1 | groupAA_CON | −0.05 | 0.36 | multivariant |
| ENSG00000132535 | DLG4 | groupAA_CON | −0.05 | 0.36 | multivariant |
| ENSG00000232022 | FAAHP1 | groupAA_CON | −0.04 | 0.36 | multivariant |
| ENSG00000183963 | SMTN | groupAA_CON | −0.04 | 0.36 | multivariant |
| ENSG00000122515 | ZMIZ2 | groupAA_CON | −0.05 | 0.36 | multivariant |
| ENSG00000189079 | ARID2 | groupAA_CON | 0.05 | 0.36 | multivariant |
| ENSG00000099795 | NDUFB7 | groupAA_CON | −0.04 | 0.36 | multivariant |
| ENSG00000063241 | ISOC2 | groupAA_CON | −0.05 | 0.36 | multivariant |
| ENSG00000125611 | CHCHD5 | groupAA_CON | −0.04 | 0.36 | multivariant |
| ENSG00000171174 | RBKS | groupAA_CON | −0.04 | 0.36 | multivariant |
| ENSG00000132612 | VPS4A | groupAA_CON | −0.06 | 0.36 | multivariant |
| ENSG00000100294 | MCAT | groupAA_CON | −0.05 | 0.36 | multivariant |
| ENSG00000112782 | CLIC5 | groupAA_CON | 0.04 | 0.36 | multivariant |
| ENSG00000104381 | GDAP1 | groupAA_CON | −0.04 | 0.36 | multivariant |
| ENSG00000156873 | PHKG2 | groupAA_CON | −0.06 | 0.37 | multivariant |
| ENSG00000143149 | ALDH9A1 | groupAA_CON | 0.06 | 0.37 | multivariant |
| ENSG00000149743 | TRPT1 | groupAA_CON | −0.05 | 0.37 | multivariant |
| ENSG00000007376 | RPUSD1 | groupAA_CON | −0.05 | 0.37 | multivariant |
| ENSG00000228300 | C19orf24 | groupAA_CON | −0.04 | 0.37 | multivariant |
| ENSG00000101868 | POLA1 | groupAA_CON | −0.04 | 0.37 | multivariant |
| ENSG00000166794 | PPIB | groupAA_CON | −0.05 | 0.37 | multivariant |
| ENSG00000183530 | PRR14L | groupAA_CON | 0.06 | 0.37 | multivariant |
| ENSG00000179091 | CYC1 | groupAA_CON | −0.05 | 0.37 | multivariant |
| ENSG00000243678 | NME2 | groupAA_CON | −0.05 | 0.37 | multivariant |
| ENSG00000197448 | GSTK1 | groupAA_CON | −0.05 | 0.37 | multivariant |
| ENSG00000112667 | DNPH1 | groupAA_CON | −0.05 | 0.37 | multivariant |
| ENSG00000166068 | SPRED1 | groupAA_CON | 0.04 | 0.37 | multivariant |
| ENSG00000178623 | GPR35 | groupAA_CON | −0.04 | 0.37 | multivariant |
| ENSG00000257702 | LBX2-AS1 | groupAA_CON | −0.04 | 0.37 | multivariant |
| ENSG00000133059 | DSTYK | groupAA_CON | 0.05 | 0.38 | multivariant |
| ENSG00000159640 | ACE | groupAA_CON | 0.03 | 0.40 | multivariant |
| ENSG00000128524 | ATP6V1F | groupAA_CON | −0.05 | 0.40 | multivariant |
| ENSG00000236778 | INTS6-AS1 | groupAA_CON | 0.04 | 0.40 | multivariant |
| ENSG00000160194 | NDUFV3 | groupAA_CON | −0.05 | 0.41 | multivariant |
| ENSG00000169627 | BOLA2B | groupAA_CON | −0.04 | 0.42 | multivariant |
| ENSG00000084623 | EIF3I | groupAA_CON | −0.05 | 0.43 | multivariant |
| ENSG00000117480 | FAAH | groupAA_CON | −0.04 | 0.46 | multivariant |
| ENSG00000065833 | ME1 | groupAA_CON | −0.03 | 0.46 | multivariant |
| ENSG00000171121 | KCNMB3 | groupAA_CON | 0.03 | 0.46 | multivariant |
| ENSG00000126106 | TMEM53 | groupAA_CON | −0.03 | 0.46 | multivariant |
| ENSG00000255274 | SMIM35 | groupAA_CON | 0.03 | 0.47 | multivariant |
| ENSG00000238197 | PAXBP1-AS1 | groupAA_CON | 0.04 | 0.48 | multivariant |
| ENSG00000196199 | MPHOSPH8 | groupAA_CON | 0.04 | 0.49 | multivariant |
| ENSG00000272356 | AL080317.3 | groupAA_CON | 0.03 | 0.49 | multivariant |
| ENSG00000119969 | HELLS | groupAA_CON | 0.02 | 0.49 | multivariant |
| ENSG00000253522 | MIR3142HG | groupAA_CON | 0.03 | 0.49 | multivariant |
| ENSG00000156172 | C8orf37 | groupAA_CON | 0.03 | 0.50 | multivariant |
| ENSG00000186792 | HYAL3 | groupAA_CON | −0.03 | 0.50 | multivariant |
| ENSG00000256594 | AC010186.2 | groupAA_CON | 0.03 | 0.50 | multivariant |
| ENSG00000023608 | SPC1 | groupAA_CON | 0.03 | 0.51 | multivariant |
| ENSG00000117560 | FASLG | groupAA_CON | 0.03 | 0.52 | multivariant |
| ENSG00000103044 | HAS3 | groupAA_CON | 0.03 | 0.53 | multivariant |
| ENSG00000233806 | LINC01237 | groupAA_CON | 0.02 | 0.54 | multivariant |
| ENSG00000285799 | AL645929.2 | groupAA_CON | 0.02 | 0.56 | multivariant |
| ENSG00000176714 | CCDC121 | groupAA_CON | 0.02 | 0.56 | multivariant |
| ENSG00000196456 | ZNF775 | groupAA_CON | −0.03 | 0.57 | multivariant |
| ENSG00000117682 | DHDDS | groupAA_CON | −0.03 | 0.58 | multivariant |
| ENSG00000134602 | STK26 | groupAA_CON | 0.02 | 0.59 | multivariant |
| ENSG00000100650 | SRSF5 | groupAA_CON | 0.03 | 0.64 | multivariant |
| ENSG00000285943 | AC112128.1 | groupAA_CON | 0.01 | 0.72 | multivariant |
| ENSG00000131725 | WDR44 | groupAA_CON | 0.02 | 0.75 | multivariant |
| ENSG00000267041 | ZNF850 | groupAA_CON | −0.01 | 0.80 | multivariant |
| ENSG00000111300 | A25 | groupAA_CON | −0.01 | 0.85 | multivariant |
| ENSG00000257242 | LINC01619 | groupAA_CON | 0.01 | 0.87 | multivariant |
| ENSG00000141753 | IGFBP4 | groupAA_CON | 0.00 | 0.95 | multivariant |
| ENSG00000198814 | GK | groupCRC_CON | 0.26 | 0.01 | univariant |
| ENSG00000138722 | MMRN1 | groupCRC_CON | 0.45 | 0.00 | univariant |
| ENSG00000112303 | VNN2 | groupCRC_CON | 0.21 | 0.02 | univariant |
| ENSG00000163220 | S100A9 | groupCRC_CON | 0.22 | 0.02 | univariant |
| ENSG00000181274 | FRAT2 | groupCRC_CON | 0.18 | 0.03 | univariant |
| ENSG00000054523 | KIF1B | groupCRC_CON | 0.17 | 0.03 | univariant |
| ENSG00000197063 | MAFG | groupCRC_CON | 0.17 | 0.03 | univariant |
| ENSG00000069399 | BCL3 | groupCRC_CON | 0.17 | 0.03 | univariant |
| ENSG00000123700 | KCNJ2 | groupCRC_CON | 0.21 | 0.02 | univariant |
| ENSG00000178999 | AURKB | groupCRC_CON | 0.14 | 0.09 | multivariant |
| ENSG00000257093 | KIAA1147 | groupCRC_CON | −0.22 | 0.01 | univariant |
| ENSG00000187837 | HIST1H1C | groupCRC_CON | 0.28 | 0.01 | univariant |
| ENSG00000259330 | INAFM2 | groupCRC_CON | 0.19 | 0.01 | univariant |
| ENSG00000198736 | MSRB1 | groupCRC_CON | 0.19 | 0.03 | univariant |
| ENSG00000268734 | AC245128.3 | groupCRC_CON | 0.20 | 0.03 | univariant |
| ENSG00000198934 | MAGEE1 | groupCRC_CON | −0.19 | 0.03 | univariant |
| ENSG00000162415 | ZSWIM5 | groupCRC_CON | −0.20 | 0.03 | univariant |
| ENSG00000172216 | CEBPB | groupCRC_CON | 0.17 | 0.04 | univariant |
| ENSG00000249437 | NAIP | groupCRC_CON | 0.19 | 0.04 | univariant |
| ENSG00000109689 | STIM2 | groupCRC_CON | −0.21 | 0.01 | univariant |
| ENSG00000259436 | AC010247.2 | groupCRC_CON | −0.18 | 0.03 | univariant |
| ENSG00000105609 | LILRB5 | groupCRC_CON | 0.16 | 0.03 | univariant |
| ENSG00000162545 | CAMK2N1 | groupCRC_CON | −0.20 | 0.03 | univariant |
| ENSG00000100385 | IL2RB | groupCRC_CON | −0.21 | 0.02 | univariant |
| ENSG00000204427 | ABHD16A | groupCRC_CON | 0.22 | 0.00 | univariant |
| ENSG00000034533 | ASTE1 | groupCRC_CON | −0.17 | 0.01 | univariant |
| ENSG00000157895 | C12orf43 | groupCRC_CON | −0.18 | 0.01 | univariant |
| ENSG00000143162 | CREG1 | groupCRC_CON | 0.31 | 0.00 | univariant |
| ENSG00000187266 | EPOR | groupCRC_CON | 0.16 | 0.03 | univariant |
| ENSG00000203618 | GP1BB | groupCRC_CON | 0.48 | 0.00 | univariant |
| ENSG00000107521 | HPS1 | groupCRC_CON | 0.18 | 0.03 | univariant |
| ENSG00000160796 | NBEAL2 | groupCRC_CON | 0.27 | 0.00 | univariant |
| ENSG00000122490 | PQLC1 | groupCRC_CON | 0.38 | 0.00 | univariant |
| ENSG00000184860 | SDR42E1 | groupCRC_CON | −0.31 | 0.01 | univariant |
| ENSG00000075399 | VPS9D1 | groupCRC_CON | 0.28 | 0.00 | univariant |
| ENSG00000132768 | DPH2 | groupCRC_CON | −0.19 | 0.02 | univariant |
| ENSG00000096746 | HNRNPH3 | groupCRC_CON | 0.09 | 0.08 | multivariant |
| ENSG00000140688 | C16orf58 | groupCRC_CON | −0.25 | 0.00 | univariant |
| ENSG00000047644 | WWC3 | groupCRC_CON | 0.21 | 0.00 | univariant |
| ENSG00000160818 | GPATCH4 | groupCRC_CON | −0.23 | 0.00 | univariant |
| ENSG00000118689 | FOXO3 | groupCRC_CON | 0.25 | 0.00 | univariant |
| ENSG00000145860 | RNF145 | groupCRC_CON | 0.22 | 0.01 | univariant |
| ENSG00000137460 | FHDC1 | groupCRC_CON | 0.33 | 0.01 | univariant |
| ENSG00000173812 | EIF1 | groupCRC_CON | 0.16 | 0.01 | univariant |
| ENSG00000110851 | PRDM4 | groupCRC_CON | −0.19 | 0.01 | univariant |
| ENSG00000059588 | TARBP1 | groupCRC_CON | −0.25 | 0.01 | univariant |
| ENSG00000187109 | NAP1L1 | groupCRC_CON | −0.16 | 0.01 | univariant |
| ENSG00000178814 | OPLAH | groupCRC_CON | 0.34 | 0.01 | univariant |
| ENSG00000183508 | TENT5C | groupCRC_CON | 0.31 | 0.01 | univariant |
| ENSG00000145882 | PCYOX1L | groupCRC_CON | −0.16 | 0.01 | univariant |
| ENSG00000141562 | NARF | groupCRC_CON | 0.20 | 0.01 | univariant |
| ENSG00000102349 | KLF8 | groupCRC_CON | −0.28 | 0.01 | univariant |
| ENSG00000178927 | CYBC1 | groupCRC_CON | 0.19 | 0.01 | univariant |
| ENSG00000145041 | DCAF1 | groupCRC_CON | −0.18 | 0.01 | univariant |
| ENSG00000185909 | KLHDC8B | groupCRC_CON | 0.27 | 0.01 | univariant |
| ENSG00000164430 | CGAS | groupCRC_CON | 0.19 | 0.01 | univariant |
| ENSG00000177383 | MAGEF1 | groupCRC_CON | −0.19 | 0.01 | univariant |
| ENSG00000105281 | SLC1A5 | groupCRC_CON | 0.24 | 0.01 | univariant |
| ENSG00000115042 | FAHD2A | groupCRC_CON | −0.19 | 0.01 | univariant |
| ENSG00000126351 | THRA | groupCRC_CON | −0.16 | 0.01 | univariant |
| ENSG00000103257 | SLC7A5 | groupCRC_CON | 0.21 | 0.01 | univariant |
| ENSG00000149016 | TUT1 | groupCRC_CON | −0.18 | 0.02 | univariant |
| ENSG00000253276 | CCDC71L | groupCRC_CON | 0.23 | 0.02 | univariant |
| ENSG00000138433 | CIR1 | groupCRC_CON | 0.16 | 0.02 | univariant |
| ENSG00000050130 | JKAMP | groupCRC_CON | 0.18 | 0.02 | univariant |
| ENSG00000114127 | XRN1 | groupCRC_CON | 0.21 | 0.02 | univariant |
| ENSG00000133193 | FAM104A | groupCRC_CON | 0.20 | 0.02 | univariant |
| ENSG00000169991 | IFFO2 | groupCRC_CON | −0.17 | 0.02 | univariant |
| ENSG00000077721 | UBE2A | groupCRC_CON | 0.13 | 0.02 | univariant |
| ENSG00000196778 | OR52K1 | groupCRC_CON | 0.24 | 0.02 | univariant |
| ENSG00000087269 | NOP14 | groupCRC_CON | −0.16 | 0.02 | univariant |
| ENSG00000101782 | RIOK3 | groupCRC_CON | 0.21 | 0.02 | univariant |
| ENSG00000005893 | LAMP2 | groupCRC_CON | 0.17 | 0.02 | univariant |
| ENSG00000114812 | VIPR1 | groupCRC_CON | −0.17 | 0.03 | univariant |
| ENSG00000177674 | AGTRAP | groupCRC_CON | 0.17 | 0.03 | univariant |
| ENSG00000119844 | AFTPH | groupCRC_CON | 0.14 | 0.03 | univariant |
| ENSG00000149483 | TMEM138 | groupCRC_CON | −0.16 | 0.03 | univariant |
| ENSG00000184574 | LPAR5 | groupCRC_CON | −0.17 | 0.03 | univariant |
| ENSG00000168781 | PPIP5K1 | groupCRC_CON | −0.20 | 0.04 | univariant |
| ENSG00000106392 | C1GALT1 | groupCRC_CON | 0.20 | 0.04 | univariant |
| ENSG00000049769 | PPP1R3F | groupCRC_CON | −0.18 | 0.05 | univariant |
| ENSG00000250318 | AC003072.1 | groupCRC_CON | 0.24 | 0.00 | univariant |
| ENSG00000119326 | CTNNAL1 | groupCRC_CON | 0.42 | 0.00 | univariant |
| ENSG00000154764 | WNT7A | groupCRC_CON | −0.32 | 0.01 | univariant |
| ENSG00000169704 | GP9 | groupCRC_CON | 0.35 | 0.01 | univariant |
| ENSG00000100568 | VTI1B | groupCRC_CON | 0.28 | 0.01 | univariant |
| ENSG00000013364 | MVP | groupCRC_CON | 0.20 | 0.01 | univariant |
| ENSG00000105738 | SIPA1L3 | groupCRC_CON | −0.31 | 0.01 | univariant |
| ENSG00000166326 | TRIM44 | groupCRC_CON | −0.16 | 0.01 | univariant |
| ENSG00000213937 | CLDN9 | groupCRC_CON | 0.26 | 0.01 | univariant |
| ENSG00000135838 | NPL | groupCRC_CON | 0.21 | 0.01 | univariant |
| ENSG00000140564 | FURIN | groupCRC_CON | 0.21 | 0.01 | univariant |
| ENSG00000168899 | VAMP5 | groupCRC_CON | 0.26 | 0.01 | univariant |
| ENSG00000129103 | SUMF2 | groupCRC_CON | −0.16 | 0.01 | univariant |
| ENSG00000136040 | PLXNC1 | groupCRC_CON | 0.23 | 0.01 | univariant |
| ENSG00000113013 | HSPA9 | groupCRC_CON | −0.15 | 0.01 | univariant |
| ENSG00000120942 | UBIAD1 | groupCRC_CON | −0.17 | 0.01 | univariant |
| ENSG00000248019 | FAM13A-AS1 | groupCRC_CON | 0.18 | 0.01 | univariant |
| ENSG00000180667 | YOD1 | groupCRC_CON | 0.30 | 0.01 | univariant |
| ENSG00000070501 | POLB | groupCRC_CON | 0.19 | 0.01 | univariant |
| ENSG00000125459 | MSTO1 | groupCRC_CON | −0.22 | 0.01 | univariant |
| ENSG00000185897 | FFAR3 | groupCRC_CON | 0.32 | 0.01 | univariant |
| ENSG00000179409 | GEMIN4 | groupCRC_CON | −0.20 | 0.01 | univariant |
| ENSG00000034713 | GABARAPL2 | groupCRC_CON | 0.19 | 0.01 | univariant |
| ENSG00000123106 | CCDC91 | groupCRC_CON | −0.30 | 0.01 | univariant |
| ENSG00000135108 | FBXO21 | groupCRC_CON | −0.21 | 0.01 | univariant |
| ENSG00000166825 | ANPEP | groupCRC_CON | 0.24 | 0.01 | univariant |
| ENSG00000232912 | AL096855.1 | groupCRC_CON | 0.24 | 0.01 | univariant |
| ENSG00000125124 | BBS2 | groupCRC_CON | −0.24 | 0.01 | univariant |
| ENSG00000164181 | ELOVL7 | groupCRC_CON | 0.27 | 0.01 | univariant |
| ENSG00000134905 | CARS2 | groupCRC_CON | 0.20 | 0.01 | univariant |
| ENSG00000086102 | NFX1 | groupCRC_CON | −0.13 | 0.01 | univariant |
| ENSG00000105255 | FSD1 | groupCRC_CON | −0.27 | 0.01 | univariant |
| ENSG00000090674 | MCOLN1 | groupCRC_CON | 0.21 | 0.01 | univariant |
| ENSG00000099985 | OSM | groupCRC_CON | 0.26 | 0.01 | univariant |
| ENSG00000158773 | USF1 | groupCRC_CON | 0.15 | 0.01 | univariant |
| ENSG00000167635 | ZNF146 | groupCRC_CON | −0.20 | 0.01 | univariant |
| ENSG00000277443 | MARCKS | groupCRC_CON | 0.21 | 0.01 | univariant |
| ENSG00000026103 | FAS | groupCRC_CON | 0.22 | 0.01 | univariant |
| ENSG00000204323 | SMIM5 | groupCRC_CON | 0.28 | 0.01 | univariant |
| ENSG00000186310 | NAP1L3 | groupCRC_CON | −0.26 | 0.01 | univariant |
| ENSG00000112335 | SNX3 | groupCRC_CON | 0.21 | 0.01 | univariant |
| ENSG00000060971 | ACAA1 | groupCRC_CON | 0.16 | 0.01 | univariant |
| ENSG00000235192 | AC009495.3 | groupCRC_CON | 0.25 | 0.01 | univariant |
| ENSG00000164649 | CDCA7L | groupCRC_CON | −0.23 | 0.01 | univariant |
| ENSG00000125245 | GPR18 | groupCRC_CON | −0.19 | 0.01 | univariant |
| ENSG00000279884 | AC010894.5 | groupCRC_CON | 0.22 | 0.01 | univariant |
| ENSG00000078618 | NRDC | groupCRC_CON | 0.19 | 0.02 | univariant |
| ENSG00000166946 | CCNDBP1 | groupCRC_CON | 0.18 | 0.02 | univariant |
| ENSG00000118600 | RXYLT1 | groupCRC_CON | −0.20 | 0.02 | univariant |
| ENSG00000213782 | DDX47 | groupCRC_CON | −0.15 | 0.02 | univariant |
| ENSG00000167074 | TEF | groupCRC_CON | −0.20 | 0.02 | univariant |
| ENSG00000160410 | SHKBP1 | groupCRC_CON | 0.23 | 0.02 | univariant |
| ENSG00000131845 | ZNF304 | groupCRC_CON | −0.20 | 0.02 | univariant |
| ENSG00000255398 | HCAR3 | groupCRC_CON | 0.21 | 0.02 | univariant |
| ENSG00000106628 | POLD2 | groupCRC_CON | −0.18 | 0.02 | univariant |
| ENSG00000116120 | FARSB | groupCRC_CON | −0.15 | 0.02 | univariant |
| ENSG00000108960 | MMD | groupCRC_CON | 0.23 | 0.02 | univariant |
| ENSG00000182134 | TDRKH | groupCRC_CON | −0.18 | 0.02 | univariant |
| ENSG00000278845 | MRPL45 | groupCRC_CON | −0.13 | 0.02 | univariant |
| ENSG00000143384 | MCL1 | groupCRC_CON | 0.16 | 0.02 | univariant |
| ENSG00000151651 | ADAM8 | groupCRC_CON | 0.18 | 0.02 | univariant |
| ENSG00000166681 | BEX3 | groupCRC_CON | 0.21 | 0.02 | univariant |
| ENSG00000186566 | GPATCH8 | groupCRC_CON | −0.18 | 0.02 | univariant |
| ENSG00000198286 | CARD11 | groupCRC_CON | −0.18 | 0.02 | univariant |
| ENSG00000204628 | RACK1 | groupCRC_CON | −0.13 | 0.02 | univariant |
| ENSG00000051128 | HOMER3 | groupCRC_CON | 0.20 | 0.02 | univariant |
| ENSG00000177666 | PNPLA2 | groupCRC_CON | 0.17 | 0.02 | univariant |
| ENSG00000133789 | SWAP70 | groupCRC_CON | −0.18 | 0.02 | univariant |
| ENSG00000130024 | PHF10 | groupCRC_CON | −0.13 | 0.02 | univariant |
| ENSG00000267121 | AC008105.3 | groupCRC_CON | 0.15 | 0.02 | univariant |
| ENSG00000174282 | ZBTB4 | groupCRC_CON | −0.15 | 0.02 | univariant |
| ENSG00000176170 | SPHK1 | groupCRC_CON | 0.21 | 0.02 | univariant |
| ENSG00000265666 | RARA-AS1 | groupCRC_CON | 0.21 | 0.02 | univariant |
| ENSG00000113460 | BRIX1 | groupCRC_CON | −0.21 | 0.02 | univariant |
| ENSG00000163938 | GNL3 | groupCRC_CON | −0.20 | 0.02 | univariant |
| ENSG00000117410 | ATP6V0B | groupCRC_CON | 0.17 | 0.02 | univariant |
| ENSG00000136929 | HEMGN | groupCRC_CON | 0.24 | 0.02 | univariant |
| ENSG00000196204 | RNF216P1 | groupCRC_CON | −0.16 | 0.02 | univariant |
| ENSG00000206344 | HCG27 | groupCRC_CON | 0.21 | 0.02 | univariant |
| ENSG00000114554 | PLXNA1 | groupCRC_CON | −0.23 | 0.02 | univariant |
| ENSG00000134851 | TMEM165 | groupCRC_CON | 0.19 | 0.02 | univariant |
| ENSG00000156471 | PTDSS1 | groupCRC_CON | −0.13 | 0.02 | univariant |
| ENSG00000155744 | FAM126B | groupCRC_CON | 0.21 | 0.02 | univariant |
| ENSG00000137414 | FAM8A1 | groupCRC_CON | 0.20 | 0.02 | univariant |
| ENSG00000168010 | ATG16L2 | groupCRC_CON | 0.16 | 0.02 | univariant |
| ENSG00000111052 | LIN7A | groupCRC_CON | 0.22 | 0.02 | univariant |
| ENSG00000271858 | CYB561D2 | groupCRC_CON | −0.22 | 0.02 | univariant |
| ENSG00000241170 | AP001992.1 | groupCRC_CON | 0.19 | 0.02 | univariant |
| ENSG00000196668 | LINC00173 | groupCRC_CON | 0.22 | 0.02 | univariant |
| ENSG00000125826 | RBCK1 | groupCRC_CON | 0.16 | 0.02 | univariant |
| ENSG00000267288 | AC138150.2 | groupCRC_CON | 0.22 | 0.02 | univariant |
| ENSG00000115806 | GORASP2 | groupCRC_CON | −0.14 | 0.02 | univariant |
| ENSG00000188636 | RTL6 | groupCRC_CON | −0.17 | 0.02 | univariant |
| ENSG00000134954 | ETS1 | groupCRC_CON | −0.18 | 0.02 | univariant |
| ENSG00000119535 | CSF3R | groupCRC_CON | 0.18 | 0.02 | univariant |
| ENSG00000065183 | WDR3 | groupCRC_CON | −0.22 | 0.02 | univariant |
| ENSG00000285693 | AP002381.2 | groupCRC_CON | 0.19 | 0.02 | univariant |
| ENSG00000173540 | GMPPB | groupCRC_CON | −0.16 | 0.02 | univariant |
| ENSG00000064547 | LPAR2 | groupCRC_CON | 0.18 | 0.02 | univariant |
| ENSG00000100211 | CBY1 | groupCRC_CON | −0.16 | 0.02 | univariant |
| ENSG00000111885 | MAN1A1 | groupCRC_CON | 0.20 | 0.02 | univariant |
| ENSG00000158201 | ABHD3 | groupCRC_CON | 0.19 | 0.02 | univariant |
| ENSG00000092010 | PSME1 | groupCRC_CON | 0.15 | 0.02 | univariant |
| ENSG00000019995 | ZRANB1 | groupCRC_CON | 0.15 | 0.02 | univariant |
| ENSG00000135966 | TGFBRAP1 | groupCRC_CON | −0.12 | 0.02 | univariant |
| ENSG00000128438 | TBC1D27P | groupCRC_CON | −0.23 | 0.02 | univariant |
| ENSG00000160602 | NEK8 | groupCRC_CON | −0.18 | 0.02 | univariant |
| ENSG00000101158 | NELFCD | groupCRC_CON | −0.16 | 0.02 | univariant |
| ENSG00000100365 | NCF4 | groupCRC_CON | 0.18 | 0.02 | univariant |
| ENSG00000125148 | MT2A | groupCRC_CON | 0.23 | 0.02 | univariant |
| ENSG00000105639 | JAK3 | groupCRC_CON | 0.15 | 0.02 | univariant |
| ENSG00000173473 | SMARCC1 | groupCRC_CON | −0.13 | 0.03 | univariant |
| ENSG00000148300 | REXO4 | groupCRC_CON | −0.13 | 0.03 | univariant |
| ENSG00000146828 | SLC12A9 | groupCRC_CON | 0.17 | 0.03 | univariant |
| ENSG00000205056 | LINC02397 | groupCRC_CON | −0.22 | 0.03 | univariant |
| ENSG00000135144 | DTX1 | groupCRC_CON | −0.22 | 0.03 | univariant |
| ENSG00000162729 | IGSF8 | groupCRC_CON | −0.15 | 0.03 | univariant |
| ENSG00000099860 | GADD45B | groupCRC_CON | 0.17 | 0.03 | univariant |
| ENSG00000007923 | DNAJC11 | groupCRC_CON | −0.13 | 0.03 | univariant |
| ENSG00000198399 | ITSN2 | groupCRC_CON | 0.17 | 0.03 | univariant |
| ENSG00000114993 | RTKN | groupCRC_CON | −0.22 | 0.03 | univariant |
| ENSG00000139631 | CSAD | groupCRC_CON | 0.19 | 0.03 | univariant |
| ENSG00000204613 | TRIM10 | groupCRC_CON | 0.22 | 0.03 | univariant |
| ENSG00000115306 | SPTBN1 | groupCRC_CON | −0.16 | 0.03 | univariant |
| ENSG00000085733 | CTTN | groupCRC_CON | 0.21 | 0.03 | univariant |
| ENSG00000244701 | AC004918.1 | groupCRC_CON | −0.18 | 0.03 | univariant |
| ENSG00000203999 | LINC01270 | groupCRC_CON | 0.22 | 0.03 | univariant |
| ENSG00000188897 | AC099489.1 | groupCRC_CON | 0.21 | 0.03 | univariant |
| ENSG00000101412 | E2F1 | groupCRC_CON | 0.21 | 0.03 | univariant |
| ENSG00000096433 | ITPR3 | groupCRC_CON | −0.17 | 0.03 | univariant |
| ENSG00000233426 | EIF3FP3 | groupCRC_CON | −0.22 | 0.03 | univariant |
| ENSG00000122642 | FKBP9 | groupCRC_CON | 0.22 | 0.03 | univariant |
| ENSG00000139146 | SINHCAF | groupCRC_CON | −0.18 | 0.03 | univariant |
| ENSG00000235437 | LINC01278 | groupCRC_CON | −0.17 | 0.03 | univariant |
| ENSG00000102796 | DHRS12 | groupCRC_CON | 0.19 | 0.03 | univariant |
| ENSG00000123091 | RNF11 | groupCRC_CON | 0.21 | 0.03 | univariant |
| ENSG00000178719 | GRINA | groupCRC_CON | 0.19 | 0.03 | univariant |
| ENSG00000196700 | ZNF512B | groupCRC_CON | −0.16 | 0.03 | univariant |
| ENSG00000132819 | RBM38 | groupCRC_CON | 0.21 | 0.03 | univariant |
| ENSG00000256806 | C17orf100 | groupCRC_CON | −0.20 | 0.03 | univariant |
| ENSG00000139644 | TMBIM6 | groupCRC_CON | 0.13 | 0.03 | univariant |
| ENSG00000188906 | LRRK2 | groupCRC_CON | 0.19 | 0.03 | univariant |
| ENSG00000172270 | BSG | groupCRC_CON | 0.20 | 0.03 | univariant |
| ENSG00000174021 | GNG5 | groupCRC_CON | 0.16 | 0.03 | univariant |
| ENSG00000166938 | DIS3L | groupCRC_CON | −0.19 | 0.03 | univariant |
| ENSG00000185340 | GAS2L1 | groupCRC_CON | 0.20 | 0.03 | univariant |
| ENSG00000083223 | TUT7 | groupCRC_CON | 0.16 | 0.03 | univariant |
| ENSG00000189430 | NCR1 | groupCRC_CON | −0.20 | 0.03 | univariant |
| ENSG00000168646 | AXIN2 | groupCRC_CON | −0.21 | 0.03 | univariant |
| ENSG00000280832 | GSEC | groupCRC_CON | 0.20 | 0.03 | univariant |
| ENSG00000113811 | SELENOK | groupCRC_CON | 0.19 | 0.03 | univariant |
| ENSG00000150938 | CRIM1 | groupCRC_CON | −0.21 | 0.03 | univariant |
| ENSG00000114423 | CBLB | groupCRC_CON | −0.17 | 0.03 | univariant |
| ENSG00000109220 | CHIC2 | groupCRC_CON | 0.15 | 0.03 | univariant |
| ENSG00000166987 | MBD6 | groupCRC_CON | 0.16 | 0.03 | univariant |
| ENSG00000198574 | SH2D1B | groupCRC_CON | −0.20 | 0.03 | univariant |
| ENSG00000112033 | PPARD | groupCRC_CON | −0.11 | 0.03 | univariant |
| ENSG00000181016 | LSMEM1 | groupCRC_CON | 0.20 | 0.03 | univariant |
| ENSG00000136193 | SCRN1 | groupCRC_CON | −0.17 | 0.03 | univariant |
| ENSG00000153814 | JAZF1 | groupCRC_CON | 0.19 | 0.03 | univariant |
| ENSG00000161405 | IKZF3 | groupCRC_CON | −0.16 | 0.03 | univariant |
| ENSG00000204576 | PRR3 | groupCRC_CON | −0.13 | 0.03 | univariant |
| ENSG00000139620 | KANSL2 | groupCRC_CON | −0.16 | 0.03 | univariant |
| ENSG00000133316 | WDR74 | groupCRC_CON | −0.15 | 0.03 | univariant |
| ENSG00000100614 | PPM1A | groupCRC_CON | 0.20 | 0.03 | univariant |
| ENSG00000102910 | LONP2 | groupCRC_CON | −0.18 | 0.03 | univariant |
| ENSG00000118096 | IFT46 | groupCRC_CON | −0.17 | 0.03 | univariant |
| ENSG00000277791 | PSMB3 | groupCRC_CON | 0.16 | 0.03 | univariant |
| ENSG00000137574 | TGS1 | groupCRC_CON | −0.14 | 0.03 | univariant |
| ENSG00000280088 | AC126474.2 | groupCRC_CON | 0.15 | 0.03 | univariant |
| ENSG00000173372 | C1QA | groupCRC_CON | 0.21 | 0.03 | univariant |
| ENSG00000111641 | NOP2 | groupCRC_CON | −0.14 | 0.03 | univariant |
| ENSG00000196923 | PDLIM7 | groupCRC_CON | 0.18 | 0.03 | univariant |
| ENSG00000114023 | FAM162A | groupCRC_CON | −0.16 | 0.03 | univariant |
| ENSG00000083093 | PALB2 | groupCRC_CON | −0.21 | 0.03 | univariant |
| ENSG00000184557 | SOCS3 | groupCRC_CON | 0.20 | 0.03 | univariant |
| ENSG00000119977 | TCTN3 | groupCRC_CON | −0.14 | 0.03 | univariant |
| ENSG00000111802 | TDP2 | groupCRC_CON | 0.16 | 0.03 | univariant |
| ENSG00000185482 | STAC3 | groupCRC_CON | 0.17 | 0.03 | univariant |
| ENSG00000116983 | HPCAL4 | groupCRC_CON | −0.20 | 0.03 | univariant |
| ENSG00000011600 | TYROBP | groupCRC_CON | 0.17 | 0.03 | univariant |
| ENSG00000059377 | TBXAS1 | groupCRC_CON | 0.16 | 0.03 | univariant |
| ENSG00000167113 | COQ4 | groupCRC_CON | −0.13 | 0.03 | univariant |
| ENSG00000138867 | GUCD1 | groupCRC_CON | 0.19 | 0.03 | univariant |
| ENSG00000118922 | KLF12 | groupCRC_CON | −0.18 | 0.03 | univariant |
| ENSG00000248769 | AC139495.2 | groupCRC_CON | 0.20 | 0.03 | univariant |
| ENSG00000148834 | GSTO1 | groupCRC_CON | 0.16 | 0.03 | univariant |
| ENSG00000250687 | AC146944.2 | groupCRC_CON | 0.20 | 0.03 | univariant |
| ENSG00000239653 | PSMD6-AS2 | groupCRC_CON | 0.19 | 0.03 | univariant |
| ENSG00000102886 | GDPD3 | groupCRC_CON | 0.16 | 0.03 | univariant |
| ENSG00000105339 | DENND3 | groupCRC_CON | 0.17 | 0.04 | univariant |
| ENSG00000197721 | CR1L | groupCRC_CON | 0.19 | 0.04 | univariant |
| ENSG00000143436 | MRPL9 | groupCRC_CON | −0.15 | 0.04 | univariant |
| ENSG00000244041 | LINC01011 | groupCRC_CON | 0.16 | 0.04 | univariant |
| ENSG00000245164 | LINC00861 | groupCRC_CON | −0.17 | 0.04 | univariant |
| ENSG00000164105 | SAP30 | groupCRC_CON | 0.18 | 0.04 | univariant |
| ENSG00000186184 | POLR1D | groupCRC_CON | 0.18 | 0.04 | univariant |
| ENSG00000205339 | IPO7 | groupCRC_CON | −0.17 | 0.04 | univariant |
| ENSG00000163534 | FCRL1 | groupCRC_CON | −0.20 | 0.04 | univariant |
| ENSG00000204519 | ZNF551 | groupCRC_CON | −0.19 | 0.04 | univariant |
| ENSG00000115594 | IL1R1 | groupCRC_CON | 0.18 | 0.04 | univariant |
| ENSG00000135119 | RNFT2 | groupCRC_CON | −0.20 | 0.04 | univariant |
| ENSG00000143382 | ADAMTSL4 | groupCRC_CON | 0.18 | 0.04 | univariant |
| ENSG00000137720 | C11orf1 | groupCRC_CON | −0.19 | 0.04 | univariant |
| ENSG00000275216 | AL161431.1 | groupCRC_CON | 0.19 | 0.04 | univariant |
| ENSG00000104524 | PYCR3 | groupCRC_CON | −0.17 | 0.04 | univariant |
| ENSG00000189046 | ALKBH2 | groupCRC_CON | −0.17 | 0.04 | univariant |
| ENSG00000182782 | HCAR2 | groupCRC_CON | 0.17 | 0.04 | univariant |
| ENSG00000183624 | HMCES | groupCRC_CON | −0.14 | 0.04 | univariant |
| ENSG00000145354 | CISD2 | groupCRC_CON | 0.19 | 0.04 | univariant |
| ENSG00000244560 | AC004890.2 | groupCRC_CON | −0.18 | 0.04 | univariant |
| ENSG00000261326 | LINC01355 | groupCRC_CON | −0.18 | 0.04 | univariant |
| ENSG00000104765 | BNIP3L | groupCRC_CON | 0.19 | 0.04 | univariant |
| ENSG00000271699 | SNX29P2 | groupCRC_CON | −0.19 | 0.04 | univariant |
| ENSG00000204475 | NCR3 | groupCRC_CON | −0.18 | 0.04 | univariant |
| ENSG00000160226 | CFAP410 | groupCRC_CON | −0.16 | 0.04 | univariant |
| ENSG00000088247 | KHSRP | groupCRC_CON | −0.13 | 0.05 | univariant |
| ENSG00000281404 | LINC01176 | groupCRC_CON | 0.17 | 0.05 | univariant |
| ENSG00000213373 | LINC00671 | groupCRC_CON | 0.18 | 0.05 | univariant |
| ENSG00000168591 | TMUB2 | groupCRC_CON | 0.12 | 0.05 | univariant |
| ENSG00000125630 | POLR1B | groupCRC_CON | −0.17 | 0.05 | univariant |
| ENSG00000107679 | PLEKHA1 | groupCRC_CON | −0.17 | 0.05 | univariant |
| ENSG00000204946 | ZNF783 | groupCRC_CON | −0.12 | 0.05 | univariant |
| ENSG00000281100 | AC105749.1 | groupCRC_CON | 0.16 | 0.05 | univariant |
| ENSG00000095319 | NUP188 | groupCRC_CON | −0.16 | 0.05 | univariant |
| ENSG00000165406 | MARCH8 | groupCRC_CON | 0.16 | 0.05 | univariant |
| ENSG00000197457 | STMN3 | groupCRC_CON | −0.14 | 0.06 | univariant |
| ENSG00000049759 | NEDD4L | groupCRC_CON | 0.17 | 0.06 | univariant |
| ENSG00000107242 | PIP5K1B | groupCRC_CON | 0.16 | 0.06 | univariant |
| ENSG00000100129 | EIF3L | groupCRC_CON | −0.15 | 0.06 | univariant |
| ENSG00000133740 | E2F5 | groupCRC_CON | −0.17 | 0.06 | univariant |
| ENSG00000128536 | CDHR3 | groupCRC_CON | −0.17 | 0.06 | univariant |
| ENSG00000165060 | FXN | groupCRC_CON | −0.14 | 0.06 | multivariant |
| ENSG00000169087 | HSPBAP1 | groupCRC_CON | 0.13 | 0.06 | multivariant |
| ENSG00000109332 | UBE2D3 | groupCRC_CON | 0.13 | 0.06 | multivariant |
| ENSG00000152439 | ZNF773 | groupCRC_CON | −0.15 | 0.06 | multivariant |
| ENSG00000225032 | AL162586.1 | groupCRC_CON | −0.17 | 0.06 | multivariant |
| ENSG00000204228 | HSD17B8 | groupCRC_CON | −0.14 | 0.06 | multivariant |
| ENSG00000181666 | HKR1 | groupCRC_CON | −0.14 | 0.06 | multivariant |
| ENSG00000186187 | ZNRF1 | groupCRC_CON | 0.16 | 0.06 | multivariant |
| ENSG00000116209 | TMEM59 | groupCRC_CON | 0.12 | 0.06 | multivariant |
| ENSG00000102710 | SUPT20H | groupCRC_CON | 0.12 | 0.06 | multivariant |
| ENSG00000141086 | CTRL | groupCRC_CON | 0.15 | 0.07 | multivariant |
| ENSG00000130816 | DNMT1 | groupCRC_CON | −0.15 | 0.07 | multivariant |
| ENSG00000161558 | TMEM143 | groupCRC_CON | −0.15 | 0.07 | multivariant |
| ENSG00000163412 | EIF4E3 | groupCRC_CON | 0.14 | 0.07 | multivariant |
| ENSG00000119599 | DCAF4 | groupCRC_CON | −0.16 | 0.07 | multivariant |
| ENSG00000090447 | TFAP4 | groupCRC_CON | −0.15 | 0.07 | multivariant |
| ENSG00000154102 | C16orf74 | groupCRC_CON | −0.15 | 0.07 | multivariant |
| ENSG00000140022 | STON2 | groupCRC_CON | 0.16 | 0.07 | multivariant |
| ENSG00000064703 | DDX20 | groupCRC_CON | −0.14 | 0.07 | multivariant |
| ENSG00000179909 | ZNF154 | groupCRC_CON | −0.16 | 0.08 | multivariant |
| ENSG00000089094 | KDM2B | groupCRC_CON | −0.13 | 0.08 | multivariant |
| ENSG00000043143 | JADE2 | groupCRC_CON | −0.12 | 0.08 | multivariant |
| ENSG00000132661 | NXT1 | groupCRC_CON | −0.12 | 0.08 | multivariant |
| ENSG00000187699 | C2orf88 | groupCRC_CON | 0.15 | 0.08 | multivariant |
| ENSG00000114166 | KAT2B | groupCRC_CON | 0.14 | 0.09 | multivariant |
| ENSG00000108582 | CPD | groupCRC_CON | 0.13 | 0.09 | multivariant |
| ENSG00000125731 | SH2D3A | groupCRC_CON | −0.13 | 0.09 | multivariant |
| ENSG00000186174 | BCL9L | groupCRC_CON | −0.14 | 0.09 | multivariant |
| ENSG00000005206 | SPPL2B | groupCRC_CON | −0.11 | 0.09 | multivariant |
| ENSG00000178921 | PFAS | groupCRC_CON | −0.14 | 0.10 | multivariant |
| ENSG00000104957 | CCDC130 | groupCRC_CON | −0.10 | 0.10 | multivariant |
| ENSG00000137497 | NUMA1 | groupCRC_CON | −0.09 | 0.10 | multivariant |
| ENSG00000129295 | LRRC6 | groupCRC_CON | 0.14 | 0.10 | multivariant |
| ENSG00000111203 | ITFG2 | groupCRC_CON | −0.13 | 0.10 | multivariant |
| ENSG00000101391 | CDK5RAP1 | groupCRC_CON | −0.12 | 0.10 | multivariant |
| ENSG00000198169 | ZNF251 | groupCRC_CON | −0.13 | 0.10 | multivariant |
| ENSG00000063587 | ZNF275 | groupCRC_CON | −0.12 | 0.11 | multivariant |
| ENSG00000131634 | TMEM204 | groupCRC_CON | −0.13 | 0.11 | multivariant |
| ENSG00000132382 | MYBBP1A | groupCRC_CON | −0.12 | 0.11 | multivariant |
| ENSG00000182472 | CAPN12 | groupCRC_CON | −0.14 | 0.11 | multivariant |
| ENSG00000143164 | DCAF6 | groupCRC_CON | 0.12 | 0.11 | multivariant |
| ENSG00000182979 | MTA1 | groupCRC_CON | −0.11 | 0.12 | multivariant |
| ENSG00000166685 | COG1 | groupCRC_CON | −0.12 | 0.12 | multivariant |
| ENSG00000240583 | AQP1 | groupCRC_CON | 0.13 | 0.12 | multivariant |
| ENSG00000243477 | NAA80 | groupCRC_CON | −0.12 | 0.12 | multivariant |
| ENSG00000244617 | ASPRV1 | groupCRC_CON | 0.13 | 0.12 | multivariant |
| ENSG00000185163 | DDX51 | groupCRC_CON | −0.11 | 0.12 | multivariant |
| ENSG00000133460 | SLC2A11 | groupCRC_CON | −0.13 | 0.13 | multivariant |
| ENSG00000143845 | ETNK2 | groupCRC_CON | −0.13 | 0.13 | multivariant |
| ENSG00000132153 | DHX30 | groupCRC_CON | −0.09 | 0.14 | multivariant |
| ENSG00000102057 | KCND1 | groupCRC_CON | 0.12 | 0.14 | multivariant |
| ENSG00000173762 | CD7 | groupCRC_CON | −0.11 | 0.16 | multivariant |
| ENSG00000027001 | MIPEP | groupCRC_CON | −0.11 | 0.18 | multivariant |
| ENSG00000284308 | C2orf81 | groupCRC_CON | −0.11 | 0.19 | multivariant |
| ENSG00000142303 | ADAMTS10 | groupCRC_CON | −0.10 | 0.22 | multivariant |
| ENSG00000140941 | MAP1LC3B | groupCRC_CON | 0.10 | 0.23 | multivariant |
| ENSG00000214022 | REPIN1 | groupCRC_CON | −0.07 | 0.25 | multivariant |
| ENSG00000146904 | EPHA1 | groupCRC_CON | −0.10 | 0.26 | multivariant |
| ENSG00000099821 | POLRMT | groupCRC_CON | −0.07 | 0.42 | multivariant |
| ENSG00000198894 | CIPC | groupCRC_CON | −0.25 | 0.00 | univariant |
| ENSG00000197620 | CXorf40A | groupCRC_CON | −0.16 | 0.01 | univariant |
| ENSG00000139629 | GALNT6 | groupCRC_CON | 0.20 | 0.02 | univariant |
| ENSG00000037897 | METTL1 | groupCRC_CON | −0.26 | 0.01 | univariant |
| ENSG00000141456 | PELP1 | groupCRC_CON | −0.32 | 0.00 | univariant |
| ENSG00000167291 | TBC1D16 | groupCRC_CON | −0.47 | 0.00 | univariant |
| ENSG00000181638 | ZFP41 | groupCRC_CON | −0.34 | 0.00 | univariant |
| ENSG00000197951 | ZNF71 | groupCRC_CON | −0.16 | 0.03 | univariant |
| ENSG00000180921 | FAM83H | groupCRC_CON | −0.13 | 0.08 | multivariant |
| ENSG00000058453 | CROCC | groupCRC_CON | −0.09 | 0.26 | multivariant |
| ENSG00000104518 | GSDMD | groupCRC_CON | 0.28 | 0.00 | univariant |
| ENSG00000158941 | CCAR2 | groupCRC_CON | −0.22 | 0.00 | univariant |
| ENSG00000132932 | ATP8A2 | groupCRC_CON | −0.38 | 0.00 | univariant |
| ENSG00000143374 | TARS2 | groupCRC_CON | −0.24 | 0.01 | univariant |
| ENSG00000159882 | ZNF230 | groupCRC_CON | 0.29 | 0.01 | univariant |
| ENSG00000103150 | MLYCD | groupCRC_CON | −0.18 | 0.01 | univariant |
| ENSG00000185507 | IRF7 | groupCRC_CON | 0.30 | 0.01 | univariant |
| ENSG00000179364 | PACS2 | groupCRC_CON | −0.17 | 0.01 | univariant |
| ENSG00000032444 | PNPLA6 | groupCRC_CON | 0.24 | 0.01 | univariant |
| ENSG00000106692 | FKTN | groupCRC_CON | −0.29 | 0.01 | univariant |
| ENSG00000182810 | DDX28 | groupCRC_CON | −0.16 | 0.01 | univariant |
| ENSG00000101191 | DIDO1 | groupCRC_CON | −0.12 | 0.01 | univariant |
| ENSG00000067365 | METTL22 | groupCRC_CON | 0.27 | 0.01 | univariant |
| ENSG00000159596 | TMEM69 | groupCRC_CON | −0.17 | 0.01 | univariant |
| ENSG00000187531 | SIRT7 | groupCRC_CON | 0.13 | 0.01 | univariant |
| ENSG00000131899 | LLGL1 | groupCRC_CON | −0.14 | 0.01 | univariant |
| ENSG00000178229 | ZNF543 | groupCRC_CON | −0.21 | 0.02 | univariant |
| ENSG00000131697 | NPHP4 | groupCRC_CON | 0.19 | 0.02 | univariant |
| ENSG00000170502 | NUDT9 | groupCRC_CON | −0.19 | 0.02 | univariant |
| ENSG00000127837 | AAMP | groupCRC_CON | −0.17 | 0.02 | univariant |
| ENSG00000149823 | VPS51 | groupCRC_CON | −0.17 | 0.02 | univariant |
| ENSG00000105447 | GRWD1 | groupCRC_CON | −0.21 | 0.02 | univariant |
| ENSG00000089597 | GANAB | groupCRC_CON | −0.14 | 0.03 | univariant |
| ENSG00000183208 | GDPGP1 | groupCRC_CON | −0.22 | 0.03 | univariant |
| ENSG00000023041 | ZDHHC6 | groupCRC_CON | 0.16 | 0.03 | univariant |
| ENSG00000284428 | IPO5P1 | groupCRC_CON | −0.18 | 0.04 | univariant |
| ENSG00000159588 | CCDC17 | groupCRC_CON | 0.33 | 0.00 | univariant |
| ENSG00000180787 | ZFP3 | groupCRC_CON | −0.28 | 0.00 | univariant |
| ENSG00000143184 | XCL1 | groupCRC_CON | −0.36 | 0.00 | univariant |
| ENSG00000161395 | PGAP3 | groupCRC_CON | −0.22 | 0.00 | univariant |
| ENSG00000164080 | RAD54L2 | groupCRC_CON | −0.23 | 0.00 | univariant |
| ENSG00000136861 | CDK5RAP2 | groupCRC_CON | 0.39 | 0.00 | univariant |
| ENSG00000236609 | ZNF853 | groupCRC_CON | −0.24 | 0.01 | univariant |
| ENSG00000181722 | ZBTB20 | groupCRC_CON | −0.31 | 0.01 | univariant |
| ENSG00000260267 | AC026471.1 | groupCRC_CON | −0.26 | 0.01 | univariant |
| ENSG00000197497 | ZNF665 | groupCRC_CON | −0.27 | 0.01 | univariant |
| ENSG00000135643 | KCNMB4 | groupCRC_CON | −0.35 | 0.01 | univariant |
| ENSG00000185838 | GNB1L | groupCRC_CON | −0.25 | 0.01 | univariant |
| ENSG00000113318 | MSH3 | groupCRC_CON | −0.19 | 0.01 | univariant |
| ENSG00000135040 | NAA35 | groupCRC_CON | −0.18 | 0.01 | univariant |
| ENSG00000143179 | UCK2 | groupCRC_CON | −0.22 | 0.01 | univariant |
| ENSG00000033170 | FUT8 | groupCRC_CON | 0.26 | 0.01 | univariant |
| ENSG00000279821 | AC145098.2 | groupCRC_CON | 0.23 | 0.01 | univariant |
| ENSG00000180089 | TMEM86B | groupCRC_CON | 0.25 | 0.01 | univariant |
| ENSG00000225342 | AC079630.1 | groupCRC_CON | 0.25 | 0.01 | univariant |
| ENSG00000164338 | UTP15 | groupCRC_CON | −0.24 | 0.01 | univariant |
| ENSG00000172932 | ANKRD13D | groupCRC_CON | 0.15 | 0.02 | univariant |
| ENSG00000100403 | ZC3H7B | groupCRC_CON | −0.14 | 0.02 | univariant |
| ENSG00000269981 | AL627309.7 | groupCRC_CON | 0.23 | 0.02 | univariant |
| ENSG00000078246 | TULP3 | groupCRC_CON | −0.18 | 0.02 | univariant |
| ENSG00000177548 | RABEP2 | groupCRC_CON | −0.18 | 0.02 | univariant |
| ENSG00000100592 | DAAM1 | groupCRC_CON | −0.25 | 0.02 | univariant |
| ENSG00000110719 | TCIRG1 | groupCRC_CON | 0.17 | 0.02 | univariant |
| ENSG00000004777 | ARHGAP33 | groupCRC_CON | −0.25 | 0.02 | univariant |
| ENSG00000245937 | LINC01184 | groupCRC_CON | −0.20 | 0.02 | univariant |
| ENSG00000116691 | MIIP | groupCRC_CON | 0.18 | 0.02 | univariant |
| ENSG00000161036 | LRWD1 | groupCRC_CON | 0.20 | 0.02 | univariant |
| ENSG00000100324 | TAB1 | groupCRC_CON | −0.14 | 0.02 | univariant |
| ENSG00000197863 | ZNF790 | groupCRC_CON | −0.24 | 0.02 | univariant |
| ENSG00000272037 | AP002907.1 | groupCRC_CON | 0.21 | 0.02 | univariant |
| ENSG00000111880 | RNGTT | groupCRC_CON | −0.15 | 0.02 | univariant |
| ENSG00000104043 | ATP8B4 | groupCRC_CON | 0.23 | 0.02 | univariant |
| ENSG00000171611 | PTCRA | groupCRC_CON | 0.23 | 0.02 | univariant |
| ENSG00000169223 | LMAN2 | groupCRC_CON | 0.13 | 0.02 | univariant |
| ENSG00000085415 | SEH1L | groupCRC_CON | −0.15 | 0.02 | univariant |
| ENSG00000203761 | MSTO2P | groupCRC_CON | −0.21 | 0.02 | univariant |
| ENSG00000170734 | POLH | groupCRC_CON | −0.16 | 0.02 | univariant |
| ENSG00000167210 | LOXHD1 | groupCRC_CON | 0.24 | 0.02 | univariant |
| ENSG00000198113 | TOR4A | groupCRC_CON | 0.16 | 0.02 | univariant |
| ENSG00000186017 | ZNF566 | groupCRC_CON | −0.24 | 0.02 | univariant |
| ENSG00000197417 | SHPK | groupCRC_CON | −0.14 | 0.02 | univariant |
| ENSG00000261888 | AC144831.1 | groupCRC_CON | 0.20 | 0.02 | univariant |
| ENSG00000051523 | CYBA | groupCRC_CON | 0.18 | 0.02 | univariant |
| ENSG00000205572 | SERF1B | groupCRC_CON | −0.22 | 0.02 | univariant |
| ENSG00000239282 | CASTOR1 | groupCRC_CON | −0.19 | 0.02 | univariant |
| ENSG00000138035 | PNPT1 | groupCRC_CON | 0.17 | 0.02 | univariant |
| ENSG00000075336 | TIMM21 | groupCRC_CON | −0.19 | 0.03 | univariant |
| ENSG00000111358 | GTF2H3 | groupCRC_CON | −0.21 | 0.03 | univariant |
| ENSG00000259956 | RBM15B | groupCRC_CON | −0.10 | 0.03 | univariant |
| ENSG00000103522 | IL21R | groupCRC_CON | −0.17 | 0.03 | univariant |
| ENSG00000176171 | BNIP3 | groupCRC_CON | −0.19 | 0.03 | univariant |
| ENSG00000100350 | FOXRED2 | groupCRC_CON | −0.22 | 0.03 | univariant |
| ENSG00000198105 | ZNF248 | groupCRC_CON | −0.21 | 0.03 | univariant |
| ENSG00000108733 | PEX12 | groupCRC_CON | −0.18 | 0.03 | univariant |
| ENSG00000006327 | TNFRSF12A | groupCRC_CON | 0.18 | 0.03 | univariant |
| ENSG00000143294 | PRCC | groupCRC_CON | −0.15 | 0.03 | univariant |
| ENSG00000070526 | ST6GALNAC1 | groupCRC_CON | −0.21 | 0.03 | univariant |
| ENSG00000185669 | SNAI3 | groupCRC_CON | 0.15 | 0.03 | univariant |
| ENSG00000142459 | EVI5L | groupCRC_CON | −0.16 | 0.03 | univariant |
| ENSG00000165233 | CARD19 | groupCRC_CON | 0.16 | 0.03 | univariant |
| ENSG00000180182 | MED14 | groupCRC_CON | −0.19 | 0.03 | univariant |
| ENSG00000182504 | CEP97 | groupCRC_CON | 0.20 | 0.03 | univariant |
| ENSG00000171823 | FBXL14 | groupCRC_CON | −0.17 | 0.03 | univariant |
| ENSG00000159131 | GART | groupCRC_CON | −0.14 | 0.03 | univariant |
| ENSG00000133114 | GPALPP1 | groupCRC_CON | −0.19 | 0.03 | univariant |
| ENSG00000128928 | IVD | groupCRC_CON | −0.15 | 0.03 | univariant |
| ENSG00000104205 | SGK3 | groupCRC_CON | 0.16 | 0.03 | univariant |
| ENSG00000152642 | GPD1L | groupCRC_CON | −0.16 | 0.03 | univariant |
| ENSG00000127418 | FGFRL1 | groupCRC_CON | −0.20 | 0.03 | univariant |
| ENSG00000157657 | ZNF618 | groupCRC_CON | −0.19 | 0.03 | univariant |
| ENSG00000167645 | YIF1B | groupCRC_CON | 0.14 | 0.03 | univariant |
| ENSG00000114126 | TFDP2 | groupCRC_CON | 0.21 | 0.03 | univariant |
| ENSG00000158792 | SPATA2L | groupCRC_CON | 0.17 | 0.03 | univariant |
| ENSG00000165521 | EML5 | groupCRC_CON | −0.19 | 0.03 | univariant |
| ENSG00000228672 | PROB1 | groupCRC_CON | −0.19 | 0.03 | univariant |
| ENSG00000133466 | C1QTNF6 | groupCRC_CON | −0.18 | 0.03 | univariant |
| ENSG00000176422 | SPRYD4 | groupCRC_CON | −0.14 | 0.03 | univariant |
| ENSG00000005469 | CROT | groupCRC_CON | −0.20 | 0.03 | univariant |
| ENSG00000184675 | AMER1 | groupCRC_CON | −0.16 | 0.03 | univariant |
| ENSG00000198355 | PIM3 | groupCRC_CON | 0.15 | 0.03 | univariant |
| ENSG00000033100 | CHPF2 | groupCRC_CON | 0.12 | 0.03 | univariant |
| ENSG00000180035 | ZNF48 | groupCRC_CON | −0.17 | 0.03 | univariant |
| ENSG00000184517 | ZFP1 | groupCRC_CON | −0.20 | 0.03 | univariant |
| ENSG00000168916 | ZNF608 | groupCRC_CON | 0.19 | 0.03 | univariant |
| ENSG00000100300 | TSPO | groupCRC_CON | 0.17 | 0.03 | univariant |
| ENSG00000266340 | AC138207.7 | groupCRC_CON | 0.15 | 0.03 | univariant |
| ENSG00000148384 | INPP5E | groupCRC_CON | −0.14 | 0.03 | univariant |
| ENSG00000135596 | MICAL1 | groupCRC_CON | 0.14 | 0.03 | univariant |
| ENSG00000268575 | AL031282.2 | groupCRC_CON | −0.18 | 0.03 | univariant |
| ENSG00000276136 | AC016957.2 | groupCRC_CON | 0.17 | 0.03 | univariant |
| ENSG00000197375 | SLC22A5 | groupCRC_CON | −0.20 | 0.03 | univariant |
| ENSG00000155858 | LSM11 | groupCRC_CON | −0.14 | 0.03 | univariant |
| ENSG00000128268 | MGAT3 | groupCRC_CON | 0.19 | 0.03 | univariant |
| ENSG00000120334 | CENPL | groupCRC_CON | −0.20 | 0.03 | univariant |
| ENSG00000181513 | ACBD4 | groupCRC_CON | −0.13 | 0.04 | univariant |
| ENSG00000270426 | AC099343.2 | groupCRC_CON | 0.18 | 0.04 | univariant |
| ENSG00000134571 | MYBPC3 | groupCRC_CON | 0.18 | 0.04 | univariant |
| ENSG00000105397 | TYK2 | groupCRC_CON | 0.16 | 0.04 | univariant |
| ENSG00000196118 | CCDC189 | groupCRC_CON | 0.18 | 0.04 | univariant |
| ENSG00000260948 | AL390195.2 | groupCRC_CON | −0.19 | 0.04 | univariant |
| ENSG00000276023 | DUSP14 | groupCRC_CON | −0.16 | 0.04 | univariant |
| ENSG00000226419 | SLC16A1-AS1 | groupCRC_CON | −0.20 | 0.04 | univariant |
| ENSG00000197121 | PGAP1 | groupCRC_CON | 0.16 | 0.04 | univariant |
| ENSG00000118412 | CASP8AP2 | groupCRC_CON | −0.19 | 0.04 | univariant |
| ENSG00000181704 | YIPF6 | groupCRC_CON | 0.18 | 0.04 | univariant |
| ENSG00000132423 | COQ3 | groupCRC_CON | −0.19 | 0.04 | univariant |
| ENSG00000008952 | SEC62 | groupCRC_CON | 0.19 | 0.04 | univariant |
| ENSG00000198040 | ZNF84 | groupCRC_CON | −0.19 | 0.04 | univariant |
| ENSG00000128000 | ZNF780B | groupCRC_CON | −0.19 | 0.04 | univariant |
| ENSG00000148498 | PARD3 | groupCRC_CON | 0.18 | 0.04 | univariant |
| ENSG00000196172 | ZNF681 | groupCRC_CON | −0.19 | 0.04 | univariant |
| ENSG00000159784 | FAM131B | groupCRC_CON | −0.19 | 0.04 | univariant |
| ENSG00000180626 | ZNF594 | groupCRC_CON | −0.19 | 0.04 | univariant |
| ENSG00000247077 | PGAM5 | groupCRC_CON | −0.12 | 0.04 | univariant |
| ENSG00000152904 | GGPS1 | groupCRC_CON | −0.14 | 0.04 | univariant |
| ENSG00000100413 | POLR3H | groupCRC_CON | −0.15 | 0.04 | univariant |
| ENSG00000170684 | ZNF296 | groupCRC_CON | −0.16 | 0.04 | univariant |
| ENSG00000280670 | CCDC163 | groupCRC_CON | −0.18 | 0.05 | univariant |
| ENSG00000198917 | SPOUT1 | groupCRC_CON | −0.15 | 0.05 | univariant |
| ENSG00000204839 | MROH6 | groupCRC_CON | 0.17 | 0.05 | univariant |
| ENSG00000171219 | CDC42BPG | groupCRC_CON | −0.17 | 0.05 | univariant |
| ENSG00000204116 | CHIC1 | groupCRC_CON | −0.17 | 0.05 | univariant |
| ENSG00000162144 | CYB561A3 | groupCRC_CON | −0.14 | 0.05 | univariant |
| ENSG00000203804 | ADAMTSL4-AS1 | groupCRC_CON | 0.17 | 0.05 | univariant |
| ENSG00000168758 | SEMA4C | groupCRC_CON | −0.14 | 0.05 | univariant |
| ENSG00000122741 | DCAF10 | groupCRC_CON | 0.17 | 0.05 | univariant |
| ENSG00000106608 | URGCP | groupCRC_CON | −0.15 | 0.06 | univariant |
| ENSG00000136014 | USP44 | groupCRC_CON | −0.15 | 0.06 | univariant |
| ENSG00000145736 | GTF2H2 | groupCRC_CON | 0.17 | 0.06 | univariant |
| ENSG00000124203 | ZNF831 | groupCRC_CON | −0.16 | 0.06 | univariant |
| ENSG00000070423 | RNF126 | groupCRC_CON | −0.10 | 0.06 | multivariant |
| ENSG00000167333 | TRIM68 | groupCRC_CON | −0.15 | 0.06 | multivariant |
| ENSG00000259953 | AL138756.1 | groupCRC_CON | 0.14 | 0.06 | multivariant |
| ENSG00000186594 | MIR22HG | groupCRC_CON | 0.16 | 0.06 | multivariant |
| ENSG00000101138 | CSTF1 | groupCRC_CON | −0.11 | 0.06 | multivariant |
| ENSG00000100297 | MCM5 | groupCRC_CON | −0.13 | 0.07 | multivariant |
| ENSG00000130653 | PNPLA7 | groupCRC_CON | −0.16 | 0.07 | multivariant |
| ENSG00000185379 | RAD51D | groupCRC_CON | −0.16 | 0.07 | multivariant |
| ENSG00000170085 | SIMC1 | groupCRC_CON | −0.14 | 0.07 | multivariant |
| ENSG00000103528 | SYT17 | groupCRC_CON | −0.16 | 0.07 | multivariant |
| ENSG00000172009 | THOP1 | groupCRC_CON | −0.13 | 0.07 | multivariant |
| ENSG00000175414 | ARL10 | groupCRC_CON | −0.15 | 0.08 | multivariant |
| ENSG00000059122 | FLYWCH1 | groupCRC_CON | −0.14 | 0.08 | multivariant |
| ENSG00000261526 | AC012615.1 | groupCRC_CON | −0.14 | 0.09 | multivariant |
| ENSG00000107816 | LZTS2 | groupCRC_CON | −0.11 | 0.09 | multivariant |
| ENSG00000008710 | PKD1 | groupCRC_CON | −0.14 | 0.09 | multivariant |
| ENSG00000213071 | LPAL2 | groupCRC_CON | −0.14 | 0.10 | multivariant |
| ENSG00000102878 | HSF4 | groupCRC_CON | 0.14 | 0.11 | multivariant |
| ENSG00000213402 | PTPRCAP | groupCRC_CON | −0.12 | 0.12 | multivariant |
| ENSG00000248099 | INSL3 | groupCRC_CON | 0.13 | 0.13 | multivariant |
| ENSG00000103253 | HAGHL | groupCRC_CON | −0.13 | 0.13 | multivariant |
| ENSG00000166133 | RPUSD2 | groupCRC_CON | −0.11 | 0.15 | multivariant |
| ENSG00000004139 | SARM1 | groupCRC_CON | −0.10 | 0.22 | multivariant |
| ENSG00000177595 | PIDD1 | groupCRC_CON | −0.06 | 0.42 | multivariant |
| ENSG00000133195 | SLC39A11 | hgAA_CON | −0.01 | 0.77 | multivariant |
| ENSG00000260401 | AP002761.4 | hgAA_CON | −0.22 | 0.05 | univariant |
| ENSG00000254681 | PKD1P5 | hgAA_CON | 0.00 | 0.88 | multivariant |
| ENSG00000138794 | CASP6 | hgAA_CON | 0.02 | 0.55 | multivariant |
| ENSG00000165118 | C9orf64 | hgAA_CON | 0.03 | 0.63 | multivariant |
| ENSG00000114383 | TUSC2 | hgAA_CON | −0.03 | 0.63 | multivariant |
| ENSG00000147364 | FBXO25 | hgAA_CON | −0.01 | 0.85 | multivariant |
| ENSG00000185499 | MUC1 | hgAA_CON | 1.07 | 0.00 | univariant |
| ENSG00000184226 | PCDH9 | hgAA_CON | 1.03 | 0.00 | univariant |
| ENSG00000283199 | C13orf46 | hgAA_CON | 0.67 | 0.01 | univariant |
| ENSG00000171574 | ZNF584 | hgAA_CON | 0.41 | 0.03 | univariant |
| ENSG00000111364 | DDX55 | hgAA_CON | 0.05 | 0.20 | multivariant |
| ENSG00000110619 | CARS | hgAA_CON | −0.03 | 0.53 | multivariant |
| ENSG00000140750 | ARHGAP17 | hgAA_CON | 0.03 | 0.55 | multivariant |
| ENSG00000275074 | NUDT18 | hgAA_CON | −0.02 | 0.62 | multivariant |
| ENSG00000135953 | MFSD9 | hgAA_CON | −0.02 | 0.63 | multivariant |
| ENSG00000108590 | MED31 | hgAA_CON | −0.02 | 0.63 | multivariant |
| ENSG00000140990 | NDUFB10 | hgAA_CON | 0.02 | 0.63 | multivariant |
| ENSG00000184164 | CRELD2 | hgAA_CON | −0.02 | 0.66 | multivariant |
| ENSG00000123329 | ARHGAP9 | hgAA_CON | −0.02 | 0.68 | multivariant |
| ENSG00000079616 | KIF22 | hgAA_CON | −0.02 | 0.69 | multivariant |
| ENSG00000169180 | XPO6 | hgAA_CON | −0.01 | 0.73 | multivariant |
| ENSG00000162910 | MRPL55 | hgAA_CON | −0.01 | 0.74 | multivariant |
| ENSG00000242294 | STAG3L5P | hgAA_CON | 0.01 | 0.75 | multivariant |
| ENSG00000101577 | LPIN2 | hgAA_CON | 0.01 | 0.75 | multivariant |
| ENSG00000080200 | CRYBG3 | hgAA_CON | 0.01 | 0.77 | multivariant |
| ENSG00000134815 | DHX34 | hgAA_CON | −0.01 | 0.77 | multivariant |
| ENSG00000203879 | GDI1 | hgAA_CON | −0.02 | 0.80 | multivariant |
| ENSG00000159674 | SPON2 | hgAA_CON | −0.01 | 0.80 | multivariant |
| ENSG00000197321 | SVIL | hgAA_CON | 0.00 | 0.88 | multivariant |
| ENSG00000132600 | PRMT7 | hgAA_CON | −0.01 | 0.89 | multivariant |
| ENSG00000145022 | TCTA | hgAA_CON | 0.01 | 0.90 | multivariant |
| ENSG00000138756 | BMP2K | hgAA_CON | 0.00 | 0.92 | multivariant |
| ENSG00000198336 | MYL4 | hgAA_CON | 0.00 | 0.94 | multivariant |
| ENSG00000060762 | MPC1 | hgAA_CON | 0.00 | 0.96 | multivariant |
| ENSG00000165792 | METTL17 | hgAA_CON | 0.00 | 0.96 | multivariant |
| ENSG00000105429 | MEGF8 | hgAA_CON | 0.00 | 0.98 | multivariant |
| ENSG00000170522 | ELOVL6 | hgAA_CON | 1.47 | 0.00 | univariant |
| ENSG00000119638 | NEK9 | hgAA_CON | 0.06 | 0.16 | multivariant |
| ENSG00000109066 | TMEM104 | hgAA_CON | −0.33 | 0.01 | univariant |
| ENSG00000146414 | SHPRH | hgAA_CON | 0.87 | 0.00 | univariant |
| ENSG00000173517 | PEAK1 | hgAA_CON | 0.89 | 0.00 | univariant |
| ENSG00000278540 | ACACA | hgAA_CON | 0.20 | 0.10 | multivariant |
| ENSG00000237440 | ZNF737 | hgAA_CON | 0.11 | 0.20 | multivariant |
| ENSG00000169093 | ASMTL | hgAA_CON | −0.03 | 0.53 | multivariant |
| ENSG00000130584 | ZBTB46 | hgAA_CON | 0.02 | 0.54 | multivariant |
| ENSG00000042429 | MED17 | hgAA_CON | 0.03 | 0.54 | multivariant |
| ENSG00000133067 | LGR6 | hgAA_CON | −0.02 | 0.58 | multivariant |
| ENSG00000267296 | CEBPA-DT | hgAA_CON | 0.02 | 0.63 | multivariant |
| ENSG00000157823 | AP3S2 | hgAA_CON | −0.01 | 0.76 | multivariant |
| ENSG00000162999 | DUSP19 | hgAA_CON | 0.82 | 0.01 | univariant |
| ENSG00000138376 | BARD1 | hgAA_CON | 0.85 | 0.01 | univariant |
| ENSG00000198440 | ZNF583 | hgAA_CON | 0.84 | 0.01 | univariant |
| ENSG00000163507 | CIP2A | hgAA_CON | 0.73 | 0.02 | univariant |
| ENSG00000091127 | PUS7 | hgAA_CON | −0.77 | 0.02 | univariant |
| ENSG00000096996 | IL12RB1 | hgAA_CON | −0.04 | 0.36 | multivariant |
| ENSG00000159479 | MED8 | hgAA_CON | −0.04 | 0.39 | multivariant |
| ENSG00000240849 | TMEM189 | hgAA_CON | −0.04 | 0.48 | multivariant |
| ENSG00000129235 | TXNDC17 | hgAA_CON | −0.03 | 0.49 | multivariant |
| ENSG00000157216 | SSBP3 | hgAA_CON | 0.03 | 0.55 | multivariant |
| ENSG00000101425 | BPI | hgAA_CON | −0.02 | 0.55 | multivariant |
| ENSG00000205323 | SARNP | hgAA_CON | −0.02 | 0.55 | multivariant |
| ENSG00000089050 | RBBP9 | hgAA_CON | 0.03 | 0.57 | multivariant |
| ENSG00000112685 | EXOC2 | hgAA_CON | 0.02 | 0.63 | multivariant |
| ENSG00000104856 | RELB | hgAA_CON | −0.02 | 0.63 | multivariant |
| ENSG00000178950 | GAK | hgAA_CON | −0.02 | 0.63 | multivariant |
| ENSG00000080845 | DLGAP4 | hgAA_CON | −0.03 | 0.63 | multivariant |
| ENSG00000161542 | PRPSAP1 | hgAA_CON | 0.03 | 0.63 | multivariant |
| ENSG00000133275 | CSNK1G2 | hgAA_CON | −0.03 | 0.63 | multivariant |
| ENSG00000134056 | MRPS36 | hgAA_CON | −0.02 | 0.66 | multivariant |
| ENSG00000213614 | HEXA | hgAA_CON | −0.03 | 0.67 | multivariant |
| ENSG00000175416 | CLTB | hgAA_CON | −0.02 | 0.67 | multivariant |
| ENSG00000285589 | AC010422.8 | hgAA_CON | 0.02 | 0.69 | multivariant |
| ENSG00000152223 | EPG5 | hgAA_CON | 0.02 | 0.69 | multivariant |
| ENSG00000121542 | SEC22A | hgAA_CON | −0.01 | 0.70 | multivariant |
| ENSG00000137776 | SLTM | hgAA_CON | 0.02 | 0.71 | multivariant |
| ENSG00000014138 | POLA2 | hgAA_CON | −0.01 | 0.76 | multivariant |
| ENSG00000065883 | CDK13 | hgAA_CON | 0.02 | 0.77 | multivariant |
| ENSG00000205903 | ZNF316 | hgAA_CON | 0.02 | 0.77 | multivariant |
| ENSG00000167972 | ABCA3 | hgAA_CON | 0.01 | 0.77 | multivariant |
| ENSG00000020129 | NCDN | hgAA_CON | 0.01 | 0.79 | multivariant |
| ENSG00000118961 | LDAH | hgAA_CON | 0.01 | 0.84 | multivariant |
| ENSG00000038532 | CLEC16A | hgAA_CON | 0.01 | 0.85 | multivariant |
| ENSG00000130741 | EIF2S3 | hgAA_CON | 0.01 | 0.85 | multivariant |
| ENSG00000169220 | RGS14 | hgAA_CON | −0.01 | 0.85 | multivariant |
| ENSG00000204231 | RXRB | hgAA_CON | −0.01 | 0.85 | multivariant |
| ENSG00000091542 | ALKBH5 | hgAA_CON | 0.01 | 0.86 | multivariant |
| ENSG00000129055 | APC13 | hgAA_CON | −0.01 | 0.89 | multivariant |
| ENSG00000129566 | TEP1 | hgAA_CON | 0.01 | 0.90 | multivariant |
| ENSG00000002822 | MAD1L1 | hgAA_CON | 0.00 | 0.93 | multivariant |
| ENSG00000109445 | ZNF330 | hgAA_CON | 0.00 | 0.97 | multivariant |
| ENSG00000187796 | CARD9 | hgAA_CON | 0.00 | 0.97 | multivariant |
| ENSG00000160753 | RUSC1 | hgAA_CON | 0.00 | 0.98 | multivariant |
| ENSG00000152784 | PRDM8 | hgAA_CON | 0.00 | 0.99 | multivariant |
| ENSG00000103202 | NME4 | hgAA_CON | 0.00 | 0.99 | multivariant |
| ENSG00000072694 | FCGR2B | hgAA_CON | 0.00 | 1.00 | multivariant |

TABLE 6

| ensemble ID | gene symbol | ensemble ID | gene symbol |
|---|---|---|---|
| ENSG00000047648 | ARHGAP6 | ENSG00000112699 | GMDS |
| ENSG00000143507 | DUSP10 | ENSG00000143545 | RAB13 |
| ENSG00000065911 | MTHFD2 | ENSG00000146670 | CDCA5 |
| ENSG00000112290 | WASF1 | ENSG00000147036 | LANCL3 |
| ENSG00000116711 | PLA2G4A | ENSG00000185168 | LINC00482 |
| ENSG00000143622 | RIT1 | ENSG00000227825 | SLC9A7P1 |
| ENSG00000163464 | CXCR1 | ENSG00000256433 | RP1-102E24.8 |
| ENSG00000169918 | OTUD7A | ENSG00000140526 | ABHD2 |
| ENSG00000214279 | SCART1 | ENSG00000008083 | JARID2 |
| ENSG00000171848 | RRM2 | ENSG00000108829 | LRRC59 |
| ENSG00000009790 | TRAF3IP3 | ENSG00000013725 | CD6 |
| ENSG00000033627 | ATP6V0A1 | ENSG00000051382 | PIK3CB |
| ENSG00000062282 | DGAT2 | ENSG00000075651 | PLD1 |
| ENSG00000067182 | TNFRSF1A | ENSG00000080371 | RAB21 |
| ENSG00000072954 | TMEM38A | ENSG00000087586 | AURKA |
| ENSG00000075420 | FNDC3B | ENSG00000095383 | TBC1D2 |
| ENSG00000094804 | CDC6 | ENSG00000110455 | ACCS |
| ENSG00000101265 | RASSF2 | ENSG00000114850 | SSR3 |
| ENSG00000102580 | DNAJC3 | ENSG00000114867 | EIF4G1 |
| ENSG00000107566 | ERLIN1 | ENSG00000115523 | GNLY |
| ENSG00000111321 | LTBR | ENSG00000117399 | CDC20 |
| ENSG00000111817 | DSE | ENSG00000118985 | ELL2 |
| ENSG00000117984 | CTSD | ENSG00000122882 | ECD |
| ENSG00000119318 | RAD23B | ENSG00000125810 | CD93 |
| ENSG00000121297 | TSHZ3 | ENSG00000131127 | ZNF141 |
| ENSG00000121807 | CCR2 | ENSG00000134287 | ARF3 |
| ENSG00000124491 | F13A1 | ENSG00000135862 | LAMC1 |
| ENSG00000129636 | ITFG1 | ENSG00000141367 | CLTC |
| ENSG00000132906 | CASP9 | ENSG00000143612 | C1orf43 |
| ENSG00000133961 | NUMB | ENSG00000146054 | TRIM7 |
| ENSG00000137177 | KIF13A | ENSG00000151176 | PLBD2 |
| ENSG00000138061 | CYP1B1 | ENSG00000161944 | ASGR2 |
| ENSG00000139890 | REM2 | ENSG00000162129 | CLPB |
| ENSG00000140939 | NOL3 | ENSG00000165280 | VCP |
| ENSG00000156639 | ZFAND3 | ENSG00000166033 | HTRA1 |
| ENSG00000163902 | RPN1 | ENSG00000166340 | TPP1 |
| ENSG00000164124 | TMEM144 | ENSG00000168528 | SERINC2 |
| ENSG00000168374 | ARF4 | ENSG00000170027 | YWHAG |
| ENSG00000173744 | AGFG1 | ENSG00000171115 | GIMAP8 |
| ENSG00000196405 | EVL | ENSG00000178726 | THBD |
| ENSG00000198053 | SIRPA | ENSG00000188725 | SMIM15 |
| ENSG00000198829 | SUCNR1 | ENSG00000196782 | MAML3 |
| ENSG00000143546 | S100A8 | ENSG00000197265 | GTF2E2 |
| ENSG00000163823 | CCR1 | ENSG00000197632 | SERPINB2 |
| ENSG00000107099 | DOCK8 | ENSG00000198900 | TOP1 |
| ENSG00000273295 | AP000350.5 | ENSG00000205038 | PKHD1L1 |
| ENSG00000173638 | SLC19A1 | ENSG00000228960 | OR2A9P |
| ENSG00000171659 | GPR34 | ENSG00000239264 | TXNDC5 |
| ENSG00000198836 | OPA1 | ENSG00000246526 | RP11- |
| | | | 539L10.2 |
| ENSG00000197353 | LYPD2 | ENSG00000254709 | IGLL5 |
| ENSG00000265354 | TIMM23 | | |

1. A method for assessing risk of, detecting, diagnosing, prognosticating, predicting, and/or monitoring colorectal neoplasia in a subject, wherein the method comprises measuring the expression level of at least one of the genes disclosed in any one of Tables 1 to 6 in a sample from the subject.

2. The method of claim 1, wherein the colorectal neoplasia is advanced adenoma (AA) or colorectal cancer (CRC).

3. The method of claim 2, wherein the advanced adenoma is AA with high grade dysplasia (AA hg).

4. The method according to claim 1 comprising the steps of:
(i) measuring the expression level in the sample from the subject;
(ii) calculating a probability score based on the measurement of step (i);
(iii) comparing the probability score calculated in (ii) with a pre-determined reference score associated to a known risk, diagnosis, prognosis, and/or prediction of colorectal neoplasia;
(iv) finding a positive or negative deviation or no deviation of the expression level measured in (i) from the pre-determined reference score; and
(v) attributing said finding of positive or negative or no deviation to a particular risk, diagnosis, prognosis, and/or prediction of colorectal neoplasia in the subject.

5. The method according to claim 1, wherein the sample is whole blood or a fractional component thereof.

6. The method according to claim 2, wherein the CRC is early CRC.

7. The method according to claim 1, wherein the method further comprises measuring in the sample or in a separate sample of the subject the presence, absence, and/or quantity of one or more additional gene useful for assessing risk of, detecting, diagnosing, prognosing, predicting, and/or monitoring colorectal neoplasia.

8. The method according to claim 1, wherein the expression level of at least one of the genes is measured by microarray expression profiling, PCR, reverse transcriptase PCR, reverse transcriptase real-time PCR, quantitative real-time PCR, digital PCR, end-point PCR, multiplex end-point PCR, mass spectrometry, in situ hybridization (ISH), multiplex ISH, next generation nucleic acid sequencing, or a combination of said methods.

9. The method according to claim 1, wherein the colorectal neoplasia is advanced adenoma (AA) or AA with high grade dysplasia (hgAA) and wherein the method comprises measuring the expression level of at least one of the genes disclosed in Table 1, Table 2, and/or Table 3.

10. The method according to claim 9, wherein the method comprises measuring the expression level of at least one of the genes disclosed in Table 1, Table 2, and/or Table 3 having an AA score superior to 2.

11. The method according to claim 9, wherein the method comprises measuring the expression level of;
i) at least one of the genes disclosed in Table 2, wherein the AA score is between 2-100; and/or
ii) at least one of the genes disclosed in Table 3, wherein the AA score is between 2-84.

12. The method according to claim 1, wherein the colorectal neoplasia is colorectal cancer (CRC) and wherein the method comprises measuring the expression level of at least one of the genes disclosed in Table 1, Table 2, Table 4, and/or Table 6.

13. The method according to claim 12, wherein the method comprises measuring the expression level of at least one of the genes disclosed in Table 2, Table 4, and/or Table 6 having a CRC score greater than or equal to 15.

14. The method according to claim 12, wherein the method comprises measuring the expression level of:
i) at least one of the genes disclosed in Table 2, wherein the CRC score is between 2-300;
ii) at least one of the genes disclosed in Table 4, wherein the CRC score is between 2-69; and/or
iii) at least one of the genes disclosed in Table 6, wherein the CRC score is between 2-101.

15. A kit for assessing risk of, detecting, diagnosing, prognosticating, predicting, and/or monitoring colorectal neoplasia comprising a means for measuring the expression level of at least one of the genes as defined in claim 1.

16. The kit according to claim 15, wherein the kit further comprises a reference control obtained from subject(s) having a known risk, diagnosis, prognosis, and/or prediction of colorectal neoplasia.

17. (canceled)

18. (canceled)

19. A device for performing a method according to claim 1, said device comprising:
i) a sample chamber for a test sample collected from the subject;
ii) an assay module in fluid communication with said sample chamber, said assay module comprising a means and/or reagents for detecting and/or measuring, directly or indirectly, the expression level of at least one of the genes disclosed in any one of Tables 1 to 6 in said test sample;
iii) a means for computing a neoplasia probability score; and
iv) a user interface wherein said user interface relates the neoplasia probability score to detecting colorectal neoplasia in said subject, stratifying colorectal neoplasia, or determining the responsiveness to a treatment.

20. A computer-implemented method for implementing the method for assessing risk of, detecting, diagnosing, prognosticating, predicting, and/or monitoring colorectal neoplasia of claim 1 in the subject, said computer-implemented method comprising:
(i) measuring the expression level in the sample from the subject;
(ii) calculating a probability score based on the measurement of step (i);
(iii) comparing the probability score calculated in step (ii) with a pre-determined reference score associated to a known risk, diagnosis, prognosis, and/or prediction of colorectal neoplasia;
(iv) finding a positive or negative deviation or no deviation of the expression level measured in step (i) from the pre-determined reference score; and
(v) attributing said finding of positive or negative or no deviation to a particular risk, diagnosis, prognosis, and/or prediction of colorectal neoplasia in the subject.

21. A method of treatment and/or prevention of colorectal neoplasia, the method comprising:
(i) performing the method for assessing risk of, detecting, diagnosing, prognosticating, predicting, and/or monitoring colorectal neoplasia of claim 1; and
(ii) administering a medical treatment or therapy aimed at treating and/or preventing said colorectal neoplasia if the results conclude that the subject is suffering from colorectal neoplasia.

22. The method of treatment of claim 21, wherein the medical treatment or therapy is a chemotherapeutic agent, a targeted therapy drug, a small-molecule drug, a monoclonal antibody, radiation therapy, immunotherapy, endoscopic treatment, and/or surgical treatment.
